(19) United States
(12) Patent Application Publication
(10) Pub. No.: US 2024/0374592 A1
(43) Pub. Date: Nov. 14, 2024

(54) METHODS AND COMPOSITIONS FOR PERTURBING MONOCYTE AND NEUTROPHIL LINEAGES

(71) Applicant: Flagship Pioneering Innovations VI, LLC, Cambridge, MA (US)

(72) Inventors: Morag Helen STEWART, Boston, MA (US); Mauricio CORTES, Wellesley, MA (US); Fabian Alexander WOLF, Munich (DE); Nicholas McCartney PLUGIS, Boston, MA (US)

(21) Appl. No.: 18/687,684

(22) PCT Filed: Sep. 14, 2022
(86) PCT No.: PCT/US2022/043513

§ 371 Date: Feb. 28, 2024

Related U.S. Application Data
(60) Provisional application No. 63/244,012, filed on Sep. 14, 2021.
(60) Provisional application No. 63/244,008, filed on Sep. 14, 2021.
(60) Provisional application No. 63/244,003, filed on Sep. 14, 2021.
(60) Provisional application No. 63/243,999, filed on Sep. 14, 2021.

(51) Int. Cl. A61K 31/506; A61K 31/166; A61K 31/167; A61K 31/18; A61K 31/341; A61K 31/35; A61K 31/365; A61K 31/4184; A61K 31/496; A61K 31/513; A61K 31/519; A61K 31/535; G01N 33/50

(52) U.S. Cl. CPC A61K 31/506; A61K 31/166; A61K 31/167; A61K 31/18; A61K 31/341; A61K 31/35; A61K 31/365; A61K 31/4184; A61K 31/496; A61K 31/513; A61K 31/519; A61K 31/535; G01N 33/5023; G01N 33/5047; C12N 2503/02

(57) ABSTRACT

The present disclosure relates, inter alia, to perturbagens and methods for directing a change or inhibiting a change in the cell state of a hematopoietic progenitor cell. It also relates to methods for increasing or decreasing a quantity of neutrophils, monocytes or immediate progenitors thereof and/or the ratios thereof. Further, the present disclosure relates to methods for treating diseases or disorders characterized by, at least, abnormal ratios of neutrophils to monocytes and/or abnormal numbers thereof.

### CROSS-REFERENCE TO RELATED APPLICATIONS

This application claims priority to and benefit of U.S. Provisional Patent Application No. 63/243,999, filed on Sep. 14, 2021, U.S. Provisional Patent Application No. 63/244,003, filed on Sep. 14, 2021, U.S. Provisional Patent Application No. 63/244,008, filed on Sep. 14, 2021, and U.S. Provisional Patent Application No. 63/244,012, filed on Sep. 14, 2021, the contents of all of which are hereby incorporated by reference in their entireties.

### BACKGROUND

The study of cellular mechanisms relating to monocyte and neutrophil lineages and methods and agents for directing or inhibiting changes in the same are useful for treating diseases or disorders characterized by, at least, abnormal ratios of neutrophils and monocytes. Currently, there is an unmet need for such methods and agents.

### SUMMARY

Accordingly, in certain aspects, the present disclosure provides methods for directing a change in the cell state of a hematopoietic progenitor cell and agents that are suitable for achieving the same (perturbagens). Also provided are methods for increasing and/or decreasing a quantity of neutrophils, monocytes or immediate progenitors thereof and/or the ratios thereof. Further provided are methods for treating diseases or disorders characterized by, at least, abnormal ratios of neutrophils to monocytes and/or abnormal numbers thereof. In various aspects, the cellular manipulations described herein are guided and/or mediated by gene signatures that reflect a cellular state and/or capacity for transitioning to a different cellular state.

An aspect of the present disclosure includes a method for directing a change in cell state of a progenitor cell comprising: contacting a population of cells comprising a progenitor cell with at least one perturbagen selected from Table 3, or a variant thereof, wherein the at least one perturbagen is capable of directing a gene signature in the progenitor cell; and wherein the progenitor cell is a non-lineage committed CD34+ cell.

An aspect of the present disclosure includes a method for directing a change in cell state of a progenitor cell, comprising: contacting a population of cells comprising a progenitor cell with at least one perturbagen capable of altering a gene signature in the progenitor cell, wherein altering the gene signature comprises an increase in expression and/or activity in the progenitor cell of one or more genes selected from Table 1 and/or a decrease in expression and/or activity in the progenitor cell of one or more genes selected from Table 2 and wherein the progenitor cell is a non-lineage committed CD34+ cell.

An aspect of the present disclosure includes a method for directing a change in cell state of a progenitor cell, comprising: contacting a population of cells comprising a progenitor cell with at least one perturbagen selected from Table 3, or a variant thereof, and capable of altering a gene signature in the progenitor cell, wherein altering the gene signature comprises an increase in expression and/or activity in the progenitor cell of one or more genes selected from Table 1 and/or a decrease in expression and/or activity in the progenitor cell of one or more genes selected from Table 2 and wherein the progenitor cell is a non-lineage committed CD34+ cell.

In some embodiments, the change in cell state provides one or more of the following: a) an increase in the number of neutrophils and/or the number of monocytes, optionally wherein the increase in the number of neutrophils and/or the number of monocytes is relative to the number of neutrophils and/or the number of monocytes obtained from a population of progenitor cells that is not contacted with the at least one perturbagen or relative to the population of progenitor cells prior to contacting with the at least one perturbagen; and/or b) the number of progenitor cells is decreased, optionally wherein the decrease in the number of progenitor cells is relative to the number of progenitor cells in a population of progenitor cells that is not contacted with the at least one perturbagen or relative to the number of progenitor cells in the population prior to contacting with the at least one perturbagen; and/or c) the number of progenitor cells is increased, optionally wherein the increase in the number of progenitor cells is relative to the number of progenitor cells in a population of progenitor cells that is not contacted with the at least one perturbagen or relative to the number of progenitor cells in the population prior to contacting with the at least one perturbagen.

In some embodiments, the number of CD66b+ neutrophil progenitors, CD64+monocytic progenitors, neutrophilic promyelocytes, neutrophilic myelocytes, neutrophilic metamyelocytes, and/or neutrophilic band cells is increased and/or the number of CD34+CD38+/− cells are increased about two days after contacting the population of cells comprising a CD34+ cell with the at least one perturbagen and/or the number of CD34-CD38+/− cells are increased about five days after contacting the population of cells comprising a CD34+ cell with the at least one perturbagen.

In some embodiments, the number of monoblasts and/or promonocytes is increased and/or the number of granulocyte-monocyte progenitor (GMP) cells is increased, optionally wherein the progenitor cell for the GMP cell is a common myeloid progenitor (CMP); and/or the number of monocyte-dendritic cell progenitor (MDP) cells is decreased.

In some embodiments, the at least one perturbagen selected from Table 3, or a variant thereof, comprises at least 2, at least 3, at least 4, at least 5, at least 6, at least 7, at least 8, at least 9, at least 10, at least 11, or 12 perturbagens selected from Table 3, or variants thereof, optionally wherein the at least one perturbagen promotes the monocyte lineage or the neutrophil lineage.

In some embodiments, altering the gene signature comprises increased expression and/or increased activity in the progenitor cell of one or more genes selected from Table 1, optionally wherein the one or more genes selected from Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, or 49 genes selected from Table 1.

In some embodiments, the one or more genes selected from Table 1 comprises at least one of MYB, NCOA4, CEBPD, CEBPA, TLE4, DACH1, SMARCA2, GFI1, MAZ, TLE1, ELL2, and ARID1A, optionally at least one of MYB, NCOA4, CEBPD, CEBPA, and TLE4.

In some embodiments, altering the gene signature comprises decreased expression and/or decreased activity in the progenitor cell of one or more genes selected from Table 2, optionally wherein the one or more genes selected from Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, or 72 genes selected from Table 2.

In some embodiments, the one or more genes selected from Table 2 comprises at least one of HMGA1, SSBP4, LSM4, CDK4, SMARCB1, AES, GTF3A, RAB8A, RNPS1, THRAP3, and SNRPB, optionally at least one of HMGA1, SSBP4, LSM4, CDK4, and SMARCB1.

An aspect of the present disclosure includes a method for inhibiting a change in cell state of a progenitor cell comprising: contacting a population of cells comprising a progenitor cell with at least one perturbagen selected from Table 6, or a variant thereof, wherein the at least one perturbagen is capable of inhibiting a gene signature in the progenitor cell; and wherein the progenitor cell is a non-lineage committed CD34+ cell.

An aspect of the present disclosure includes a method for inhibiting a change in cell state of a progenitor cell, comprising: contacting a population of cells comprising a progenitor cell with at least one perturbagen capable of altering a gene signature in the progenitor cell, wherein inhibiting the gene signature comprises an increase in expression and/or activity in the progenitor cell of one or more genes selected from Table 4 and/or a decrease in expression and/or activity in the progenitor cell of one or more genes selected from Table 5 and wherein the progenitor cell is a non-lineage committed CD34+ cell.

An aspect of the present disclosure includes a method for inhibiting a change in cell state of a progenitor cell, comprising: contacting a population of cells comprising a progenitor cell with at least one perturbagen selected from Table 6, or a variant thereof, and capable of altering a gene signature in the progenitor cell, wherein inhibiting the gene signature comprises an increase in expression and/or activity in the progenitor cell of one or more genes selected from Table 4 and/or a decrease in expression and/or activity in the progenitor cell of one or more genes selected from Table 5 and wherein the progenitor cell is a non-lineage committed CD34+ cell.

In some embodiments, inhibiting the change in cell state provides one or more of the following: a) a decrease in the number of neutrophils and/or the number of monocytes, optionally wherein the decrease in the number of neutrophils and/or the number of monocytes is relative to the number of neutrophils and/or the number of monocytes obtained from a population of progenitor cells that is not contacted with the at least one perturbagen or relative to the population of progenitor cells prior to contacting with the at least one perturbagen; and/or b) the number of progenitor cells is increased, optionally wherein the increase in the number of progenitor cells is relative to the number of progenitor cells in a population of progenitor cells that is not contacted with the at least one perturbagen or relative to the number of progenitor cells in the population prior to contacting with the at least one perturbagen.

In some embodiments, the number of CD66b+ neutrophil progenitors, CD64+monocytic progenitors, neutrophilic promyelocytes, neutrophilic myelocytes, neutrophilic metamyelocytes, and/or neutrophilic band cells is decreased and/or the number of CD34+CD38+/− cells is decreased about two days after contacting the population of cells comprising a CD34+ cell with the at least one perturbagen and/or the number of CD34-CD38+/− cells is decreased about five days after contacting the population of cells comprising a CD34+ cell with the at least one perturbagen.

In some embodiments, the number of monoblasts and/or promonocytes is decreased and/or the number of granulocyte-monocyte progenitor (GMP) cells is decreased, optionally wherein the progenitor cell for the GMP cell is a common myeloid progenitor (CMP) and/or the number of monocyte-dendritic cell progenitor (MDP) cells is increased.

In some embodiments, the at least one perturbagen selected from Table 6, or a variant thereof, comprises at least 2, at least 3, at least 4, at least 5, at least 6, at least 7, at least 8, at least 9, or 10 perturbagens selected from Table 6, or variants thereof.

In some embodiments, the at least one perturbagen prevents progression of a progenitor cell into and/or reduces the likelihood that a progenitor cell will progress into the monocyte lineage or the neutrophil lineage.

In some embodiments, altering the gene signature comprises increased expression and/or increased activity in the progenitor cell of one or more genes selected from Table 4, optionally wherein the one or more genes selected from Table 4 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, or 72 genes selected from Table 4.

In some embodiments, the one or more genes selected from Table 4 comprises at least one of HMGA1, SSBP4, LSM4, CDK4, SMARCB1, AES, GTF3A, RAB8A, RNPS1, THRAP3, and SNRPB, optionally at least one of HMGA1, SSBP4, LSM4, CDK4, and SMARCB1.

In some embodiments, altering the gene signature comprises decreased expression and/or decreased activity in the progenitor cell of one or more genes selected from Table 5, optionally wherein the one or more genes selected from Table 5 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, or 49 genes selected from Table 5.

In some embodiments, the one or more genes selected from Table 5 comprises at least one of MYB, NCOA4, CEBPD, CEBPA, TLE4, DACH1, SMARCA2, GFI1, MAZ, TLE1, ELL2, and ARID1A, optionally at least one of MYB, NCOA4, CEBPD, CEBPA, and TLE4.

In some embodiments, contacting the population of progenitor cells occurs in vitro or ex vivo or in vivo in a subject.

An aspect of the present disclosure includes a perturbagen for use in a method of the instant disclosure.

An aspect of the present disclosure includes a pharmaceutical composition comprising a perturbagen of the instant disclosure.

An aspect of the present disclosure includes a method for promoting the formation of a neutrophil, a monocyte, or an immediate progenitor thereof, comprising: (a) exposing a starting population of stem/progenitor cells comprising a non-lineage committed CD34+ cell to a perturbagen; wherein the exposing promotes the transition of the starting population of stem/progenitor cells into a granulocyte monocyte progenitor, neutrophil progenitor, monocyte progenitor or other lineage associated progenitor thereof, or (b) exposing a starting population of stem/progenitor cells comprising a non-lineage committed CD34+ cell to a perturbation having a perturbation signature that promotes the transition of the starting population of stem/progenitor cells into a granulocyte monocyte progenitor, neutrophil progenitor, monocyte progenitor or other lineage associated progenitor thereof, wherein the perturbation signature comprises increased expression and/or activity of one or more of genes selected from Table 1 and/or a decreased expression and/or activity in the non-lineage committed CD34+ cell of one or more genes selected from Table 2.

An aspect of the present disclosure includes a method for inhibiting the formation of a neutrophil, a monocyte, or an immediate progenitor thereof, comprising: (a) exposing a starting population of stem/progenitor cells comprising a non-lineage committed CD34+ cell to a perturbation having a perturbation signature that prevents progression of a progenitor cell into and/or or reduces the likelihood that a progenitor cell will progress into a granulocyte monocyte progenitor, neutrophil progenitor, monocyte progenitor or other lineage associated progenitor thereof, wherein the perturbation signature comprises increased expression and/or activity of one or more of genes selected from Table 4 and/or a decreased expression and/or activity in the non-lineage committed CD34+ cell of one or more genes selected from Table 5; or (b) exposing a starting population of stem/progenitor cells comprising a non-lineage committed CD34+ cell to a perturbagen that prevents progression of a progenitor cell into and/or or reduces the likelihood that a progenitor cell will progress into a granulocyte monocyte progenitor, neutrophil progenitor, monocyte progenitor or other lineage associated progenitor thereof.

An aspect of the present disclosure includes a method of increasing a quantity of neutrophils, monocytes, or immediate progenitors thereof, comprising: exposing a starting population of stem/progenitor cells comprising a non-lineage committed CD34+ cell to a pharmaceutical composition that promotes the formation of lineage specific progenitor population selected from granulocyte monocyte progenitor, neutrophil progenitor, monocyte progenitor, or other lineage-associated progenitors, the pharmaceutical composition promoting the transition of a primitive stem/progenitor population into the lineage specific progenitor population that has the capacity to differentiate into neutrophils, monocytes or immediate progenitors thereof, wherein the pharmaceutical composition comprises at least one perturbagen selected from Table 3, or a variant thereof.

An aspect of the present disclosure includes a method of reducing the quantity of neutrophils, monocytes, or immediate progenitors thereof in a population of cells, comprising: exposing a starting population of stem/progenitor cells comprising a non-lineage committed CD34+ cell to a pharmaceutical composition that prevents progression of a progenitor cell into and/or reduces the likelihood that a progenitor cell will progress into a lineage specific progenitor population selected from granulocyte monocyte progenitor, neutrophil progenitor, monocyte progenitor, or other lineage-associated progenitors, the pharmaceutical composition inhibiting the transition of a primitive stem/progenitor population into the lineage specific progenitor population that naturally would have the capacity to differentiate into neutrophils, monocytes or immediate progenitors thereof, wherein the pharmaceutical composition comprises at least one perturbagen selected from Table 6, or a variant thereof.

An aspect of the present disclosure includes a method for treating a disease or disorder characterized by an abnormal ratio of neutrophils to monocytes, comprising: (a) administering to a patient in need thereof a therapeutically effective amount of at least one perturbagen selected from Table 3, or a variant thereof, wherein the at least one perturbagen is capable of changing a gene signature in a progenitor cell; (b) administering to a patient in need thereof a cell, the cell having been contacted with at least one perturbagen selected from Table 3, or a variant thereof, wherein the at least one perturbagen is capable of changing a gene signature in a progenitor cell; (c) administering to a patient in need thereof a therapeutically effective amount of at least one perturbagen selected from Table 3, or a variant thereof, (d) administering to a patient in need thereof a cell, the cell having been contacted with at least one perturbagen selected from Table 3, or a variant thereof; (e) administering to a patient in need thereof a therapeutically effective amount of at least one perturbagen selected from Table 6, or a variant thereof, wherein the at least one perturbagen is capable of changing a gene signature in a progenitor cell or (f) administering to a patient in need thereof a cell, the cell having been contacted with at least one perturbagen selected from Table 6, or a variant thereof, wherein the at least one perturbagen is capable of changing a gene signature in a progenitor cell; (g) administering to a patient in need thereof a therapeutically effective amount of at least one perturbagen selected from Table 6, or a variant thereof, or (h) administering to a patient in need thereof a cell, the cell having been contacted with at least one perturbagen selected from Table 6, or a variant thereof.

In some embodiments, the abnormal ratio comprises an increased number of neutrophils and/or a decreased number of monocytes, or a decreased number of neutrophils and/or an increased number of monocytes, or a decreased number of neutrophils and a decreased number of monocytes.

In some embodiments, the abnormal ratio comprises an increased number of neutrophils and/or an increased number of monocytes.

An aspect of the present disclosure includes a method for treating a disease or disorder characterized by an abnormal ratio of neutrophils and/or monocytes to progenitor cells, comprising: (a) administering to a patient in need thereof at least one perturbagen selected from Table 3, or a variant thereof, wherein the at least one perturbagen is capable of changing a gene signature in a progenitor cell or (b) administering to a patient in need thereof a cell, the cell having been contacted with at least one perturbagen selected from Table 3, or a variant thereof, wherein the at least one perturbagen is capable of changing a gene signature in a progenitor cell.

In some embodiments, the abnormal ratio comprises a decreased number of neutrophils and/or a decreased number of monocytes and/or an increased number of progenitor cells; or a decreased number of neutrophils and an increased number of progenitor cells; or a decreased number of monocytes and an increased number of progenitor cells; or a decreased number of neutrophils and a decreased number of monocytes; or an increased number of progenitor cells.

In some embodiments, the abnormal ratio comprises an increased number of neutrophils and/or an increased number of monocytes and/or a decreased number of progenitor cells, optionally wherein the abnormal ratio comprises an increased number of neutrophils and an increased number of monocytes

In some embodiments, the disease or disorder characterized by an abnormal ratio of neutrophils to monocytes and/or characterized by an abnormal ratio of neutrophils and/or monocytes to progenitor cells is selected from leukopenia, leukocytosis, agranulocytosis, granulocytosis, monocytopenia, monocytosis, neutropenia, and neutrophilia, leukemia (e.g., Chronic myeloid leukemia (CML), Chronic myelomonocytic leukemia (CMML), and Acute myeloblastic leukemia (AML), including AML-M5), cancer induced neutropenia, drug induced neutropenia, infection induce neutropenia, lupus (and other autoimmune disorders showing neutropenia), myelofibrosis, Chronic idiopathic neutropenia (CIN), and/or Autoimmune idiopathic neutropenia (AlN).

In some embodiments, wherein the disease or disorder characterized by an abnormal ratio of neutrophils to monocytes and/or characterized by an abnormal ratio of neutrophils and/or monocytes to progenitor cells is selected from leukocytosis, granulocytosis, monocytosis, and neutrophilia, leukemia (e.g., Chronic myeloid leukemia (CML), Chronic myelomonocytic leukemia (CMML), and Acute myeloblastic leukemia (AML), including AML-M5), cancer induced leukocytosis, drug induced leukocytosis, infection induced leukocytosis, lupus (and other autoimmune disorders showing leukocytosis), and myeloproliferative neoplasms (including polycythemia vera and myelofibrosis).

In some embodiments, the disease or disorder characterized by an abnormal ratio of neutrophils to monocytes and/or characterized by an abnormal ratio of neutrophils and/or monocytes to progenitor cells is related to an autoimmune disease, a bone-marrow transplant, a cancer, a genetic disease, a bacterial infection, a viral infection, inflammatory bowel disease, tissue necrosis (e.g., myocardial infarction and burns), a previous or concurrent anti-cancer treatment, and/or, a previous or concurrent immune suppressive treatment.

An aspect of the present disclosure includes a method for treating monocytopenia, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

An aspect of the present disclosure includes a method for treating monocytosis, comprising: (a) administering to a patient in need thereof a therapeutically effective amount of at least one perturbagen selected from Table 3, or a variant thereof; (b) administering to a patient in need thereof a cell, the cell having been contacted with at least one perturbagen selected from Table 3, or a variant thereof; (c) administering to a patient in need thereof a therapeutically effective amount of at least one perturbagen selected from Table 6, or a variant thereof; or (d) administering to a patient in need thereof a cell, the cell having been contacted with at least one perturbagen selected from Table 6, or a variant thereof.

An aspect of the present disclosure includes a method for treating neutropenia, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

An aspect of the present disclosure includes a method for treating neutrophilia, comprising: (a) administering to a patient in need thereof a therapeutically effective amount of at least one perturbagen selected from Table 3, or a variant thereof; (b) administering to a patient in need thereof a cell, the cell having been contacted with at least one perturbagen selected from Table 3, or a variant thereof; (c) administering to a patient in need thereof a therapeutically effective amount of at least one perturbagen selected from Table 6, or a variant thereof; or (d) administering to a patient in need thereof a cell, the cell having been contacted with at least one perturbagen selected from Table 6, or a variant thereof.

An aspect of the present disclosure includes a method for treating agranulocytosis, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof, optionally wherein the agranulocytosis is monocytopenia or neutropenia.

An aspect of the present disclosure includes a method for treating granulocytosis, comprising: (a) administering to a patient in need thereof a therapeutically effective amount of at least one perturbagen selected from Table 3, or a variant thereof; (b) administering to a patient in need thereof a cell, the cell having been contacted with at least one perturbagen selected from Table 3, or a variant thereof, optionally wherein the agranulocytosis is monocytopenia or neutropenia; (c) administering to a patient in need thereof a therapeutically effective amount of at least one perturbagen selected from Table 6, or a variant thereof; or (d) administering to a patient in need thereof a cell, the cell having been contacted with at least one perturbagen selected from Table 6, or a variant thereof.

An aspect of the present disclosure includes a method for treating a myeloproliferative neoplasm, comprising: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

In some embodiments, the at least one perturbagen is capable of changing a gene signature in a progenitor cell

An aspect of the present disclosure includes a method of treating a disorder selected from drug-induced neutropenia, a side-effect of bone marrow transplantation, or MonoMAC syndrome, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

In some embodiments, the therapeutically effective amount of the at least one perturbagen provides a change in a gene signature in a cell, wherein the change in the gene signature comprises an increase in expression and/or activity in the cell of one or more genes selected from Table 1 and/or a decrease in expression and/or activity in the cell of one or more genes selected from Table 2 and/or an increase in expression and/or activity in the cell of one or more genes selected from Table 4 and/or a decrease in expression and/or activity in the cell of one or more genes selected from Table 5.

In some embodiments, the therapeutically effective amount of the at least one perturbagen provides an increase in the number of neutrophils and/or the number of monocytes in a sample from the human subject relative to the number of neutrophils and/or the number of monocytes in a sample from the human subject prior to administering the at least one perturbagen.

In some embodiments, the therapeutically effective amount of the at least one perturbagen provides a decrease in the number of neutrophils and/or the number of monocytes in a sample from the human subject relative to the number of neutrophils and/or the number of monocytes in a sample from the human subject prior to administering the at least one perturbagen.

In some embodiments, the patient was selected by steps comprising: (a) obtaining from the patient having the disease or disorder a sample of cells comprising a non-lineage committed CD34+ cell; and contacting the sample of cells with least one perturbagen selected from Table 3 or Table 6, or a variant thereof wherein the at least one perturbagen alters a gene signature in the sample of cells; or (b) obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein the at least one perturbagen increases in the sample of cells the expression and/or activity of one or more genes selected from Table 1 and/or decreases in the sample of cells the expression and/or activity of one or more genes selected from Table 2 and/or increases in the sample of cells the expression and/or activity of one or more genes selected from Table 4 and/or decreases in the sample of cells the expression and/or activity of one or more genes selected from Table 5; or (c) obtaining from a subject having the disease or disorder a sample of cells comprising a non-lineage committed CD34+ cell; and contacting the sample of cells with at least one perturbagen selected from Table 3 or Table 6, or a variant thereof; wherein the at least one perturbagen increases in the sample of cells the expression and/or activity of one or more genes selected from Table 1 and/or decreases in the sample of cells the expression and/or activity of one or more genes selected from Table 2; or increases in the sample of cells the expression and/or activity of one or more genes selected from Table 4 and/or decreases in the sample of cells the expression and/or activity of one or more genes selected from Table 5.

An aspect of the present disclosure includes a method for selecting the patient of any one of the methods of the instant disclosure comprising: obtaining from a subject having the disease or disorder a sample of cells comprising a non-lineage committed CD34+ cell; and contacting the sample of cells with least one perturbagen selected from Table 3 or Table 6, or a variant thereof, wherein when the at least one perturbagen alters a gene signature in the sample of cells, the subject is selected as a patient.

An aspect of the present disclosure includes a method for selecting the patient of any one of the methods of the instant disclosure comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein when the at least one perturbagen increases in the sample of cells the expression and/or activity of one or more genes selected from Table 1 and/or decreases in the sample of cells the expression and/or activity of one or more genes selected from Table 2 and/or increases in the sample of cells the expression and/or activity of one or more genes selected from Table 4 and/or decreases in the sample of cells the expression and/or activity of one or more genes selected from Table 5, the subject is selected as a patient.

An aspect of the present disclosure includes a method for selecting the patient of any one of the methods of the instant disclosure comprising: obtaining from a subject having the disease or disorder a sample of cells comprising a non-lineage committed CD34+ cell; and contacting the sample of cells with at least one perturbagen selected from Table 3 or Table 6, or a variant thereof; wherein when the at least one perturbagen increases in the sample of cells the expression and/or activity of one or more genes selected from Table 1 and/or decreases in the sample of cells the expression and/or activity of one or more genes selected from Table 2; or increases in the sample of cells the expression and/or activity of one or more genes selected from Table 4 and/or decreases in the sample of cells the expression and/or activity of one or more genes selected from Table 5, the subject is selected as a patient.

In some embodiments, the patient was selected by steps comprising: (a) obtaining from the patient having the disease or disorder a sample of cells comprising a non-lineage committed CD34+ cell; and contacting the sample of cells with least one perturbagen selected from Table 3, or a variant thereof, wherein the at least one perturbagen increases the number of monocytes and/or neutrophils or a progenitor thereof in a culture from the sample of cells; or (b) obtaining from a subject having the disease or disorder a sample of cells comprising a non-lineage committed CD34+ cell; and contacting the sample of cells with least one perturbagen selected from Table 3, or a variant thereof, wherein when the at least one perturbagen increases the number of monocytes and/or neutrophils or a progenitor thereof in a culture from the sample of cells, the subject is selected as a patient.

In some embodiments, the patient was selected by steps comprising: (a) obtaining from the patient having the disease or disorder a sample of cells comprising a non-lineage committed CD34+ cell; and contacting the sample of cells with least one perturbagen selected from Table 6, or a variant thereof, wherein the at least one perturbagen increases the number of monocytes and/or neutrophils or a progenitor thereof in a culture from the sample of cells; or (b) obtaining from a subject having the disease or disorder a sample of cells comprising a non-lineage committed CD34+ cell; and contacting the sample of cells with least one perturbagen selected from Table 6, or a variant thereof, wherein when the at least one perturbagen increases the number of monocytes and/or neutrophils or a progenitor thereof in a culture from the sample of cells, the subject is selected as a patient.

An aspect of the present disclosure includes use of a perturbagen of Table 3 or Table 6, or a variant thereof in the manufacture of a medicament for treating a disease or disorder characterized by an abnormal ratio of neutrophils to monocytes.

An aspect of the present disclosure includes use of a perturbagen of Table 3 or Table 6, or a variant thereof in the manufacture of a medicament for treating a disease or disorder characterized by an abnormal ratio of neutrophils and/or monocytes to progenitor cells.

An aspect of the present disclosure includes a method of identifying a candidate perturbation for promoting the transition of a starting population of progenitor cells into neutrophils, monocytes or immediate progenitors thereof, the method comprising: (a) exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into neutrophils, monocytes or immediate progenitors thereof following exposure of the population of cells to the perturbation; and identifying the perturbation as a candidate perturbation for promoting the transition of a population of progenitor cells into neutrophils, monocytes or immediate progenitors thereof based on the perturbation signature, wherein the perturbation signature is an increase in expression and/or activity in the progenitor cell of one or more genes selected from Table 1, and/or a decrease in expression and/or activity in the progenitor cell of one or more genes selected from Table 2; or (b) exposing the starting population of progenitor cells to a candidate perturbagen; determining a change in cell state of the cells in the population of progenitor cells into neutrophils, monocytes or immediate progenitors thereof following exposure of the population of cells to the perturbagen wherein the candidate perturbagen capable changing the cell state of the cells in the population progenitor cells is identified as a perturbagen for promoting the transition of a starting population of progenitor cells into neutrophils, monocytes or immediate progenitors thereof.

An aspect of the present disclosure includes a method of identifying a candidate perturbation for inhibiting the transition of a progenitor cell into a neutrophil, monocyte or immediate progenitor thereof, the method comprising: exposing a starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and preventing progression of a progenitor cell into and/or reducing the likelihood that a progenitor cell will progress into a neutrophil, monocyte or immediate progenitor thereof following exposure of the population of progenitor cells to the perturbation; and identifying the perturbation as a candidate perturbation for inhibiting the transition of a progenitor cell into a neutrophil, monocyte or immediate progenitor thereof based on the perturbation signature, wherein the perturbation signature is an increase in expression and/or activity in the progenitor cell of one or more genes selected from Table 4, and/or a decrease in expression and/or activity in the progenitor cell of one or more genes selected from Table 5.

An aspect of the present disclosure includes a method of identifying a perturbagen, the method comprising: exposing a starting population of progenitor cells, which naturally would have the capacity to undergo a change in cell state comprising differentiating into neutrophils, monocytes and/or immediate progenitors thereof, to a candidate perturbagen; determining an inhibition in the natural capacity to differentiate into neutrophils, monocytes and/or immediate progenitors thereof following exposure of the population of cells to the perturbagen; wherein the candidate perturbagen capable of inhibiting the natural capacity of a progenitor cell to differentiate into a neutrophil, monocyte and/or immediate progenitor thereof is identified as a perturbagen.

An aspect of the present disclosure includes a method for making a therapeutic agent for a disease or disorder selected from leukocytosis, granulocytosis, monocytosis, and neutrophilia, leukemia, cancer induced leukocytosis, drug induced leukocytosis, infection induce leukocytosis, lupus (and other autoimmune disorders showing leukocytosis), and myeloproliferative neoplasms (including polycythemia vera and myelofibrosis), comprising: (a) identifying a perturbagen capable of acting as a therapeutic agent for therapy according to a method of the instant disclosure and (b) formulating the therapeutic agent for the treatment of the disease or disorder.

An aspect of the present disclosure includes a method for making a therapeutic agent for a disease or disorder selected from leukopenia, leukocytosis, agranulocytosis, granulocytosis, monocytopenia, monocytosis, neutropenia, and neutrophilia, leukemia, cancer induced neutropenia, drug induced neutropenia, infection induce neutropenia, lupus (and other autoimmune disorders showing neutropenia), myelofibrosis, Chronic idiopathic neutropenia (CIN), and Autoimmune idiopathic neutropenia (AlN), comprising: (a) identifying a therapeutic agent for therapy according to a method of the instant disclosure and (b) formulating the therapeutic agent for the treatment of the disease or disorder.

An aspect of the present disclosure includes a method for making a therapeutic agent for a disease or disorder selected from leukocytosis, granulocytosis, monocytosis, and neutrophilia, leukemia, cancer induced leukocytosis, drug induced leukocytosis, infection induce leukocytosis, lupus (and other autoimmune disorders showing leukocytosis), and myeloproliferative neoplasms (including polycythemia vera and myelofibrosis), comprising: (a) identifying a therapeutic agent for therapy according to a method of the instant disclosure and (b) formulating the therapeutic agent for the treatment of the disease or disorder.

An aspect of the present disclosure includes a method for directing a change in cell state of a progenitor cell, comprising: contacting a population of cells comprising a progenitor cell with at least one perturbagen selected from Table 3, or a variant thereof, and capable of altering a gene signature in the progenitor cell, wherein altering the gene signature comprises an increase in expression and/or activity in the progenitor cell of one or more genes selected from Table 1 and/or a decrease in expression and/or activity in the progenitor cell of one or more genes selected from Table 2 and wherein the progenitor cell is a non-lineage committed CD34+ cell; and the change in cell state provides an increase in the number of CD66b+ neutrophil progenitors and an increase in the number of CD64+monocytic progenitors.

In some embodiments, the increase in the number of CD66b+ neutrophil progenitors and the increase in the number of CD64+monocytic progenitors is relative to the population of progenitor cells not contacted with the at least one perturbagen.

In some embodiments, the one or more genes selected from Table 1 comprises at least one of MYB, NCOA4, CEBPD, CEBPA, and TLE4.

In some embodiments, the one or more genes selected from Table 2 comprises at least one of HMGA1, SSBP4, LSM4, CDK4, and SMARCB1.

An aspect of the present disclosure includes a method for inhibiting a change in cell state of a progenitor cell, comprising: contacting a population of cells comprising a progenitor cell with at least one perturbagen selected from Table 6, or a variant thereof, and capable of altering a gene signature in the progenitor cell, wherein inhibiting the gene signature comprises an increase in expression and/or activity in the progenitor cell of one or more genes selected from Table 4 and/or a decrease in expression and/or activity in the progenitor cell of one or more genes selected from Table 5 and wherein the progenitor cell is a non-lineage committed CD34+ cell, and the change in cell state provides a decrease in the number of CD66b+ neutrophil progenitors and CD64+monocytic progenitors.

In some embodiments, the decrease in the number of CD66b+ neutrophil progenitors and the decrease in the number of CD64+monocytic progenitors is relative to the number of progenitor cells in a population of progenitor cells that is not contacted with the at least one perturbagen.

In some embodiments, the one or more genes selected from Table 4 comprises at least one of HMGA1, SSBP4, LSM4, CDK4, and SMARCB1.

In some embodiments, the one or more genes selected from Table 5 comprises at least one of MYB, NCOA4, CEBPD, CEBPA, and TLE4.

### BRIEF DESCRIPTION OF THE DRAWINGS

FIG. 1A is schematic showing lineage directions of human hematopoiesis. Each numbered cluster represents cells of a specific state/lineage. Cluster 10 represents non-lineage committed CD34+ cells and cluster 9 represents cells in the granulocyte-monocyte progenitor (GMP) lineages, which ultimately differentiate into monocytes and neutrophils. The arrow shows the effect of perturbagens that drive cells from the non-lineage committed CD34+ cells (of cluster 10) towards cells of the GMP lineages (cluster 9). Cluster 8 represent cells of the early erythroid lineage, cluster 15 represent cells of the megakaryocyte lineage, and cluster 13 represents cells of the mast cell/basophil lineage.

FIG. 1B illustrates a process for identifying and characterizing perturbagens that drive non-lineage committed CD34+ cells towards the GMP lineages (bottom left) or away from the GMP lineages (bottom right).

FIG. 1C illustrates an analysis in which progenitor cells are provided control treatments or a cocktail of cytokines (perturbagens) which drive specific cell lineage fates. NT control is the no treatment control. MK stands for megakaryocyte.

FIG. 2A and FIG. 2B shows consequences of perturbagens that promote the granulocyte-monocyte lineages. The data in FIG. 2A shows that one perturbagen (Perturbagen 2) promotes the neutrophil lineage and the data in FIG. 2B shows that two perturbagens (Perturbagen 1 and Perturbagen 4) promote the monocyte lineage. Perturbagens are listed in Table 3.

FIG. 3A and FIG. 3B shows consequences of perturbagens that promote the granulocyte-monocyte lineages. The data in FIG. 3A shows that four perturbagens (Perturbagen 1, Perturbagen 2, Perturbagen 3, and Perturbagen 4) inhibited the neutrophil lineage and the data in FIG. 3B shows that two perturbagens (Perturbagen 3 and Perturbagen 4) also inhibited the monocyte lineage. Perturbagens are listed in Table 6.

### DETAILED DESCRIPTION

The present disclosure is based, in part, on the discovery that cells of hematopoietic lineages comprising neutrophils and/or monocytes and their progenitors can be characterized by specific gene signatures. Additionally, the present disclosure is based on the discovery that certain active agents (i.e., perturbagens) can alter these specific gene signatures, which alteration is associated with the acquisition of specific cell states by the progenitors and cells of neutrophil and/or monocyte lineages. In embodiments, certain active agents (i.e., perturbagens) can promote the acquisition of specific cell states by progenitor cells and cells of neutrophil and/or monocyte lineages. The present disclosure is also based on the discovery that certain active agents (i.e., perturbagens) can alter these specific gene signatures, which alteration is associated with preventing progression of a progenitor cell into and/or reducing the likelihood that a progenitor cell will progress into cells of the neutrophil and/or monocyte lineage. In embodiments, certain active agents (i.e., perturbagens) can inhibit the acquisition of specific cell states by progenitor cells and cells of neutrophil and/or monocyte lineages. For example, the perturbagen is capable of inhibiting a progenitor cell's natural capacity to differentiate into neutrophils, monocytes and/or immediate progenitors thereof. These perturbagens are, at least, useful in therapeutics that derive a benefit from directing the progenitors towards the neutrophil and/or monocyte states and/or derive a benefit from inhibiting progenitors from acquiring the neutrophil and/or monocyte states.

### Methods for Promoting a Change in Cell State

### Genes Signatures

Cell state transitions (i.e., a transition in a cell's state from a first cell state to a second cell state, e.g., differentiation) are characterized by a change in expression of genes in the cell. Changes in gene expression may be quantified as the increase in mRNA expressed for a specific gene or a decrease in mRNA expressed for another specific gene; especially significant here may be mRNAs that encode transcription factors. Collectively, the sum of multiple differences in gene expression in one cell type or cells of one lineage relative to another cell type or cells of another lineage are referred to herein as a gene signature.

Any one of a number of methods and metrics may be used to identify gene signatures. Non-limiting examples include single cell and bulk RNA sequencing with or without prior cell sorting (e.g., fluorescence activated cell sorting (FACS) and flow cytometry). When developing a gene signature, it may useful to first characterize the cell type or cells of a specific lineage by surface proteins that are characteristic of the cell type or cells of a specific lineage. Illustrative surface proteins are listed in Table 9 and Table 11.

Knowing the gene signature for each cell type or cells of a specific lineage provides insight into what genes impact or are associated with the process of transition to other cell types and/or differentiation of progenitor cells.

Gene signatures can be used to identify particular cells as being on-lineage, and other cells as being “progenitor” cells or intermediate cells along a transition trajectory towards the on-lineage cell type.

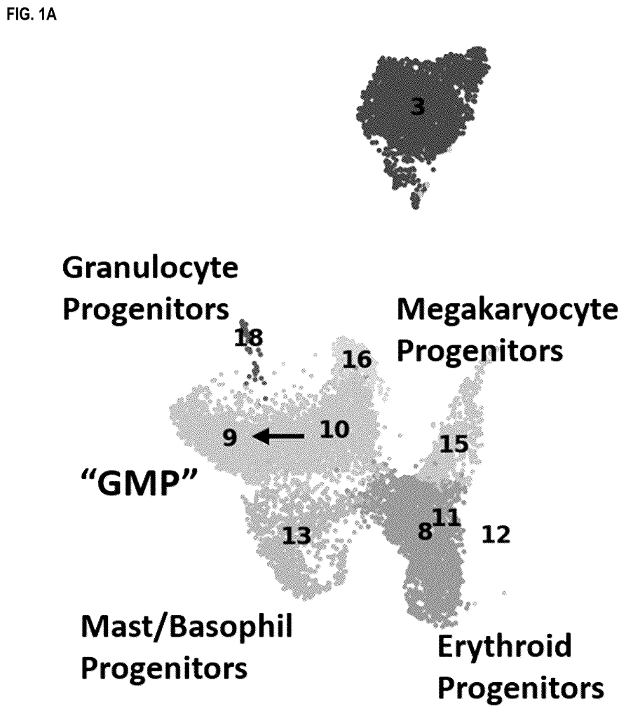

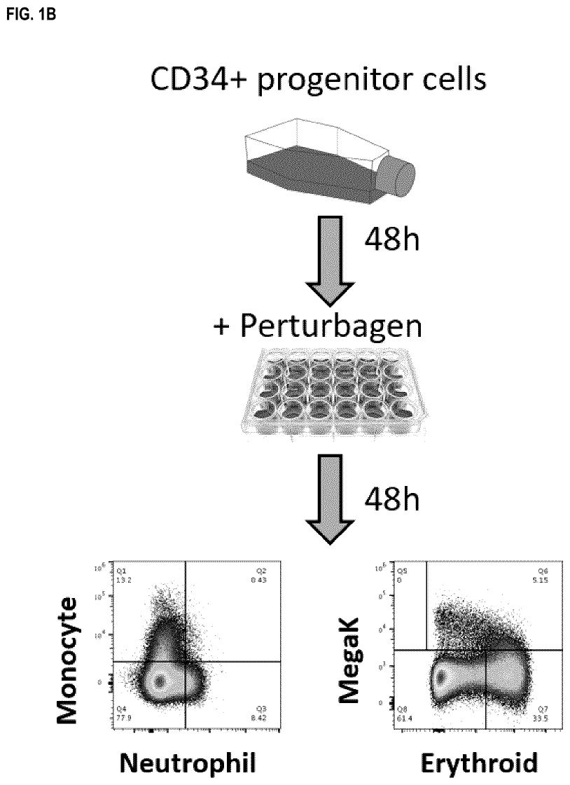

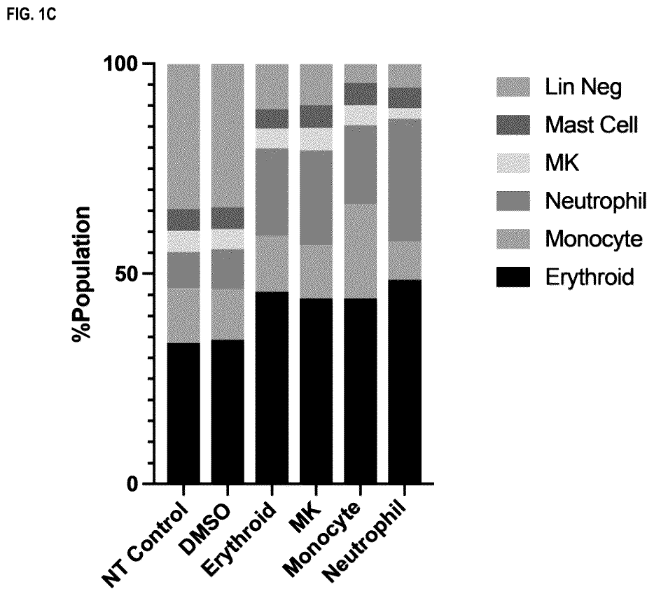

FIG. 1A, shows annotated clusters that associate gene signature with cell types or cells of a specific lineage. Differential gene signatures for the 10 to 9 transition, i.e., from a non-lineage committed CD34+ progenitor cell to cells of the granulocyte monocyte progenitor (GMP) lineage, were used to predict perturbations that would promote the transition. Those genes that are differentially expressed and positively associated with the promotion of granulocyte monocyte progenitor (GMP) lineage progression and/or GMP differentiation and negatively associated with the blocking of GMP lineage progression and/or GMP differentiation are listed in Table 1:

TABLE 1

| | Gene Name | GeneID |
|---|---|---|
| 1 | MYB | 4602 |
| 2 | NCOA4 | 8031 |
| 3 | CEBPD | 1052 |
| 4 | CEBPA | 1050 |
| 5 | TLE4 | 7091 |
| 6 | DACH1 | 1602 |
| 7 | SMARCA2 | 6595 |
| 8 | GFI1 | 2672 |
| 9 | MAZ | 4150 |
| 10 | TLE1 | 7088 |
| 11 | ELL2 | 22936 |
| 12 | ARID1A | 8289 |
| 13 | CITED4 | 163732 |
| 14 | ARID3A | 1820 |
| 15 | PTMA | 5757 |
| 16 | STAT2 | 6773 |
| 17 | NFIL3 | 4783 |
| 18 | SERTAD2 | 9792 |
| 19 | RCOR1 | 23186 |
| 20 | BTBD11 | 121551 |
| 21 | TRERF1 | 55809 |
| 22 | NOTCH2 | 4853 |
| 23 | CCNT1 | 904 |
| 24 | UHRF2 | 115426 |
| 25 | ASB1 | 51665 |
| 26 | BASP1 | 10409 |
| 27 | FOXK1 | 221937 |
| 28 | MAPK1 | 5594 |
| 29 | CREB1 | 1385 |
| 30 | IRX3 | 79191 |
| 31 | STAT4 | 6775 |
| 32 | SCX | 642658 |
| 33 | HIPK2 | 28996 |
| 34 | EZH1 | 2145 |
| 35 | ANK1 | 286 |
| 36 | ENPP2 | 5168 |
| 37 | KLF4 | 9314 |
| 38 | BCL6 | 604 |
| 39 | UTF1 | 8433 |
| 40 | NFKB1 | 4790 |
| 41 | PPP3CB | 5532 |
| 42 | RUNX1 | 861 |
| 43 | NCOA3 | 8202 |
| 44 | CITED2 | 10370 |
| 45 | RNF38 | 152006 |
| 46 | MAGED1 | 9500 |
| 47 | TNFAIP3 | 7128 |
| 48 | ZFPM1 | 161882 |
| 49 | HIVEP1 | 3096 |

At the time of filing the present disclosure, the World Wide Web at nobi.nlm.nih.gov/gene provides a description of and the nucleic acid sequence for each GeneID listed in Table 1; the contents of each of which is incorporated herein by reference in its entirety.

In contrast, those genes that are differentially expressed and positively associated with the blocking of promoting GMP lineage progression and/or GMP differentiation are listed in Table 2:

TABLE 2

| | Gene Name | GeneID |
|---|---|---|
| 1 | HMGA1 | 3159 |
| 2 | SSBP4 | 170463 |
| 3 | LSM4 | 25804 |
| 4 | CDK4 | 1019 |
| 5 | SMARCB1 | 6598 |
| 6 | AES | 166 |
| 7 | GTF3A | 2971 |
| 8 | RAB8A | 4218 |
| 9 | RNPS1 | 10921 |
| 10 | THRAP3 | 9967 |
| 11 | SNRPB | 6628 |
| 12 | CARHSP1 | 23589 |
| 13 | DEK | 7913 |
| 14 | DNMT1 | 1786 |
| 15 | POLR2J | 5439 |
| 16 | DEAF1 | 10522 |
| 17 | PHF5A | 84844 |
| 18 | E2F4 | 1874 |
| 19 | IFI16 | 3428 |
| 20 | SSRP1 | 6749 |
| 21 | PSMC5 | 5705 |
| 22 | HHEX | 3087 |
| 23 | BTF3 | 689 |
| 24 | LMO2 | 4005 |
| 25 | HMG20B | 10362 |
| 26 | HOXA9 | 3205 |
| 27 | POLR2C | 5432 |
| 28 | POLR2K | 5440 |
| 29 | LYL1 | 4066 |
| 30 | MYBL2 | 4605 |
| 31 | SAP18 | 10284 |
| 32 | TFDP2 | 7029 |
| 33 | SMARCA4 | 6597 |
| 34 | POLR21 | 5438 |
| 35 | PFDN1 | 5201 |
| 36 | MLLT3 | 4300 |
| 37 | TAF10 | 6881 |
| 38 | DRAP1 | 10589 |
| 39 | GATA2 | 2624 |
| 40 | ABT1 | 29777 |
| 41 | TAL 1 | 6886 |
| 42 | HDAC3 | 8841 |
| 43 | GFI1B | 8328 |
| 44 | TFDP1 | 7027 |
| 45 | NACA | 4666 |
| 46 | NCOR1 | 9611 |
| 47 | PDLIM1 | 9124 |
| 48 | PDCD7 | 10081 |
| 49 | HSF1 | 3297 |
| 50 | ZDHHC16 | 84287 |
| 51 | TFAM | 7019 |
| 52 | TIAL 1 | 7073 |
| 53 | ETS2 | 2114 |
| 54 | MEF2C | 4208 |
| 55 | BAZ1B | 9031 |
| 56 | NR1H2 | 7376 |
| 57 | PSMC3 | 5702 |
| 58 | HOXA7 | 3204 |
| 59 | MYC | 4609 |
| 60 | UBTF | 7343 |
| 61 | PTGES2 | 80142 |
| 62 | KEAP1 | 9817 |
| 63 | NMI | 9111 |
| 64 | FHL2 | 2274 |
| 65 | E2F1 | 1869 |
| 66 | TRIP6 | 7205 |
| 67 | TAF6 | 6878 |
| 68 | SOX4 | 6659 |
| 69 | STRAP | 11171 |
| 70 | SSBP2 | 23635 |
| 71 | ARID5B | 84159 |
| 72 | HSBP1 | 3281 |

At the time of filing the present disclosure, the World Wide Web at ncbi.nlm.nih.gov/gene provides a description of and the nucleic acid sequence for each GeneID listed in Table 2; the contents of each of which is incorporated herein by reference in its entirety.

### Perturbagens

A perturbagen useful in the present disclosure can be a small molecule, a biologic, a protein, a nucleic acid, such as a cDNA over-expressing a wild-type gene or an mRNA encoding a wild-type gene, or any combination of any of the foregoing. Illustrative perturbagens useful in the present disclosure and capable of promoting granulocyte monocyte progenitor (GMP) lineage differentiation are listed in Table 3.

TABLE 3

| | | Molecular | Effective in | |
|---|---|---|---|---|
| Perturbagen | | formula | vitro | |
| No. | Molecular weight | (g/mol) | concentration | |
| 1 | C₂₂H₂₇ClFN₇O₄S | 540 | 10.0 | um |
| 2 | C₁₃H₈Cₗ₂N₂O₄ | 327.12 | 10.0 | um |
| 3 | C₁₇H₁₅BrClFN₄O₃ | 457.7 | 10.0 | um |
| 4 | C₄₄H₆₈O₁₃ | 805.0 | 1.0 | um |
| 5 | C₂₁ H₂₃N₃O₅ | 397.4 | 10.0 | um |
| 6 | C₂₀H₁₉F₃IN₃O₅ | 565.3 | 3.33 | um |
| 7 | C₁₄H₂₀N₂O₃ | 264.32 | 10 | μM |
| 8 | C₁₆H₁₄F₃IN₂O₄ | 482.19 | 0.37 | um |
| 9 | C₂₈H₃₉N₇O₃ | 521.7 | 10 | μM |
| 10 | C₁₉H₂₀F₃IN₂O₅S | 572.3 | 10.0 | um |
| 11 | C₁₇H₂₂N₂O₃ | 302.37 | 10 | μM |
| 12 | C₃₀H₄₂O₇ | 514.6 | 10 | μM |

In various embodiments herein, a perturbagen encompasses the perturbagens named in Table 3. Thus, the named perturbagens of Table 3 represent examples of perturbagens of the present disclosure.

In Table 3, the effective in vitro concentration is the concentration of a perturbagen that is capable of increasing gene expression in a progenitor cell and/or initiating steps associated with promoting a change in cell state of a progenitor cell towards a cell of the GMP lineage, as assayed, at least, by single cell gene expression profiling (GEP). Although the concentrations were determined in an in vitro assay, the concentrations may be relevant to a determination of in vivo dosages.

In embodiments, a perturbagen used in the present disclosure is a variant of a perturbagen of Table 3. A variant may be a derivative, analog, enantiomer or a mixture of enantiomers thereof or a pharmaceutically acceptable salt, solvate, hydrate, co-crystal, clathrate, or polymorph of the perturbagen of Table 3. A variant of a perturbagen of Table 3 retains the biological activity of the perturbagen of Table 3.

### Methods and Perturbagens for Directing a Change in Cell State

Particular cellular changes in cell state can be matched to differential gene expression (which collectively define a gene signature), caused by exposure of a cell to a perturbagen. A change in cell state may be from one progenitor cell type to another progenitor cell type. For example, a common myeloid progenitor (CMP) may change to a granulocyte monocyte progenitor (GMP). A change in cell state may be from an upstream progenitor cell to a downstream progenitor cell. Lastly, a change in cell state may be from the final non-differentiated cell into a differentiated cell, e.g., a neutrophil and a monocyte.

As disclosed herein, particular cellular changes in cell state are caused by exposure of a cell to a perturbagen. A change in cell state may be from one progenitor cell type to another progenitor cell type. For example, a common myeloid progenitor (CMP) may change to a granulocyte monocyte progenitor (GMP). A change in cell state may be from an upstream progenitor cell to a downstream progenitor cell. Lastly, a change in cell state may be from the final non-differentiated cell into a differentiated cell, e.g., a neutrophil and a monocyte.

An aspect of the present disclosure is a method for directing a change in cell state of a non-lineage committed CD34+ progenitor cell. The method comprising a step of contacting a population of cells comprising a non-lineage committed CD34+ progenitor cell with at least one perturbagen selected from Table 3, or a variant thereof. In this aspect, the at least one perturbagen is capable of directing a change in cell state of a progenitor cell, which is a non-lineage committed CD34+ cell.

An aspect of the present disclosure is a method for directing a change in cell state of a non-lineage committed CD34+ progenitor cell. The method comprising a step of contacting a population of cells comprising a non-lineage committed CD34+ progenitor cell with at least one perturbagen selected from Table 3, or a variant thereof. In this aspect, the at least one perturbagen is capable of altering a gene signature in the progenitor cell.

Another aspect of the present disclosure is a method for directing a change in cell state of a non-lineage committed CD34+ progenitor cell. The method comprising a step of contacting a population of cells comprising a non-lineage committed CD34+ progenitor cell with at least one perturbagen capable of altering a gene signature in the progenitor cell. In this aspect, altering the gene signature comprises an increase in expression and/or activity in the progenitor cell of one or more genes selected from Table 1 and/or a decrease in expression and/or activity in the progenitor cell of one or more genes selected from Table 2.

Yet another aspect of the present disclosure is a method for directing a change in cell state of a non-lineage committed CD34+ progenitor cell. The method comprising a step of contacting a population of cells comprising a non-lineage committed CD34+ progenitor cell with at least one perturbagen selected from Table 3, or a variant thereof, and capable of altering a gene signature in the progenitor cell. In this aspect, altering the gene signature comprises an increase in expression and/or activity in the progenitor cell of one or more genes selected from Table 1 and/or a decrease in expression and/or activity in the progenitor cell of one or more genes selected from Table 2.

In embodiments, the non-lineage committed CD34+ cell is a hematopoietic stem and progenitor cell (HSPC).

In embodiments, the change in cell state provides an increase in the number of neutrophils and/or the number of monocytes.

In embodiments, the increase in the number of neutrophils and/or the number of monocytes is relative to the number of neutrophils and/or the number of monocytes obtained from a population of progenitor cells that is not contacted with the at least one perturbagen.

In embodiments, the increase in the number of neutrophils and/or the number of monocytes is relative to the population of progenitor cells prior to contacting with the at least one perturbagen.

In embodiments, the change in cell state provides an increase in the number of neutrophils. In embodiments, the change in cell state provides an increase in the number of monocytes. In embodiments, the change in cell state does not provide a substantial increase in the number of monocytes and/or provides a decrease in the number of monocytes. In embodiments, the ratio of the number of neutrophils to the number of monocytes is increased relative to the ratio obtained from a population of progenitor cells that is not contacted with the at least one perturbagen. In embodiments, the ratio of the number of neutrophils to the number of monocytes is increased relative to the ratio in the population of progenitor cells prior to contacting with the at least one perturbagen.

In embodiments, the change in cell state provides an increase in the number of monocytes. In embodiments, the change in cell state provides an increase in the number of neutrophils. In embodiments, the change in cell state does not provide a substantial increase in the number of neutrophils and/or provides a decrease in the number of neutrophils. In embodiments, the ratio of the number of monocytes to the number of neutrophils is increased relative to the ratio obtained from a population of progenitor cells that is not contacted with the at least one perturbagen. In embodiments, the ratio of the number of monocytes to the number of neutrophils is increased relative to the ratio in the population of progenitor cells prior to contacting with the at least one perturbagen.

In embodiments, the ratio of the number of neutrophils to the number of progenitor cells is increased relative to the ratio obtained from a population of progenitor cells that is not contacted with the at least one perturbagen.

In embodiments, the ratio of the number of neutrophils to the number of progenitor cells is increased relative to the ratio in the population of progenitor cells prior to contacting with the at least one perturbagen.

In embodiments, the ratio of the number of monocytes to the number of progenitor cells is increased relative to the ratio obtained from a population of progenitor cells that is not contacted with the at least one perturbagen.

In embodiments, the ratio of the number of monocytes to the number of progenitor cells is increased relative to the ratio in the population of progenitor cells prior to contacting with the at least one perturbagen.

In embodiments, the increase in the number of neutrophils and/or the number of monocytes is due in part to increased cell proliferation of the neutrophils and/or the monocytes.

In embodiments, the increase in the number of neutrophils and/or the number of monocytes is due in part to an increased lifespan of the neutrophils and/or the monocytes. In embodiments, the increase in the number of neutrophils and/or the number of monocytes is due in part to reduced cell death among the neutrophils and/or the monocytes. Methods for determining the extension of the lifespan of a specific cell type or a reduction of cell death is well known in the art. As examples, markers for dying cells, e.g., caspases can be detected, or dyes for dead cells, e.g., methylene blue, may be used.

In embodiments, the increase in the number of neutrophils and/or the number of monocytes is due in part to a change of cell state from progenitor cells into the neutrophil and/or monocyte lineage.

In embodiments, the number of progenitor cells is decreased. In embodiments, the decrease in the number of progenitor cells is due in part to decreased cell proliferation of the progenitor cells. In embodiments, the decrease in the number of progenitor cells is due in part to a decreased lifespan of the progenitor cells. In embodiments, the decrease in the number of progenitor cells is due in part to increased cell death among the progenitor cells. In embodiments, the decrease in the number of progenitor cells is relative to the number of progenitor cells in a population of progenitor cells that is not contacted with the at least one perturbagen. In embodiments, the decrease in the number of progenitor cells is relative to the number of progenitor cells in the population prior to contacting with the at least one perturbagen. In embodiments, the decrease in the number of progenitor cells is due to a change of cell state from a progenitor cell into the neutrophil lineage and/or monocyte lineage.

In embodiments, the number of progenitor cells is increased. In embodiments, the increase in the number of progenitor cells is due in part to increased cell proliferation of the progenitor cells. In embodiments, the increase in the number of progenitor cells is due in part to an increased lifespan of the progenitor cells. In embodiments, the increase in the number of progenitor cells is due in part to decreased cell death among the progenitor cells. In embodiments, the increase in the number of progenitor cells is relative to the number of progenitor cells in a population of progenitor cells that is not contacted with the at least one perturbagen. In embodiments, the increase in the number of progenitor cells is relative to the number of progenitor cells in the population prior to contacting with the at least one perturbagen.

Methods for counting cells are well known in the art. Non-limiting examples include hemocytometry, flow cytometry, and cell sorting techniques, e.g., fluorescence activated cell sorting (FACS).

In embodiments, the number of CD66b+ neutrophil progenitors, CD64+monocytic progenitors, neutrophilic promyelocytes, neutrophilic myelocytes, neutrophilic metamyelocytes, and/or neutrophilic band cells is increased and/or the number of CD34+CD38+/− cells are increased about two days after contacting the population of cells comprising a CD34+ cell with the at least one perturbagen and/or the number of CD34-CD38+/− cells are increased about five days after contacting the population of cells comprising a CD34+ cell with the at least one perturbagen. In embodiments, the ratio of the number of CD66b+ neutrophil progenitors, CD64+monocytic progenitors, neutrophilic promyelocytes, neutrophilic myelocytes, neutrophilic metamyelocytes, neutrophilic band cells, CD34+CD38+/− cells, and/or CD34-CD38+/− cells to the number of progenitor cells is increased relative to the ratio obtained from a population of progenitor cells that is not contacted with the at least one perturbagen. In embodiments, the ratio of the number of CD66b+ neutrophil progenitors, CD64+monocytic progenitors, neutrophilic promyelocytes, neutrophilic myelocytes, neutrophilic metamyelocytes, neutrophilic band cells, CD34+CD38+/− cells, and/or CD34-CD38+/− cells to the number of progenitor cells is increased relative to the ratio in the population of progenitor cells prior to contacting with the at least one perturbagen.

In embodiments, the number of monoblasts and/or promonocytes is increased. In embodiments, the ratio of the number of monoblasts and/or promonocytes to the number of progenitor cells is increased relative to the ratio obtained from a population of progenitor cells that is not contacted with the at least one perturbagen. In embodiments, the ratio of the number of monoblasts and/or promonocytes to the number of progenitor cells is increased relative to the ratio in the population of progenitor cells prior to contacting with the at least one perturbagen.

In embodiments, the number of GMP cells is increased. In embodiments, the ratio of the number of GMP cells to the number of progenitor cells for the GMP cell is increased relative to the ratio obtained from a population of progenitor cells that is not contacted with the at least one perturbagen. In embodiments, the ratio of the number of GMP cells to the number of progenitor cells for the GMP cell is increased relative to the ratio in the population of progenitor cells prior to contacting with the at least one perturbagen. In embodiments, the progenitor cell for the GMP cell is a common myeloid progenitor (CMP). In embodiments, the number of monocyte-dendritic cell progenitor (MDP) cells is decreased. In embodiments, the ratio of the number of GMP cells to the number of MDP cells is increased relative to the ratio obtained from a population of progenitor cells that is not contacted with the at least one perturbagen. In embodiments, the ratio of the number of GMP cells to the number of MDP cells is increased relative to the ratio in the population of progenitor cells prior to contacting with the at least one perturbagen.

In embodiments, the at least one perturbagen selected from Table 3, or a variant thereof, comprises at least 2, at least 3, at least 4, at least 5, at least 6, at least 7, at least 8, at least 9, at least 10, at least 11, or 12 perturbagens selected from Table 3, or variants thereof.

In any of the aspects or embodiments disclosed herein, the at least one perturbagen is selected from Table 3.

In embodiments, the at least one perturbagen comprises one or more perturbagens selected from Table 3, or variants thereof. In embodiments, the at least one perturbagen promotes the monocyte lineage. In embodiments, the at least one perturbagen promotes the neutrophil lineage.

In embodiments, altering the gene signature comprises increased expression and/or increased activity in the progenitor cell of one or more genes selected from Table 1. In embodiments, the one or more genes selected from Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, or 49 genes selected from Table 1. In embodiments, the one or more genes selected from Table 1 comprises at least one of MYB, NCOA4, CEBPD, CEBPA, TLE4, DACH1, SMARCA2, GFI1, MAZ, TLE1, ELL2, and ARID1A. In embodiments, the one or more genes selected from Table 1 comprises at least one of MYB, NCOA4, CEBPD, CEBPA, and TLE4.

In embodiments, altering the gene signature comprises decreased expression and/or decreased activity in the progenitor cell of one or more genes selected from Table 2. In embodiments, the one or more genes selected from Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, or 72 genes selected from Table 2. In embodiments, the one or more genes selected from Table 2 comprises at least one of HMGA1, SSBP4, LSM4, CDK4, SMARCB1, AES, GTF3A, RAB8A, RNPS1, THRAP3, and SNRPB. In embodiments, the one or more genes selected from Table 2 comprises at least one of HMGA1, SSBP4, LSM4, CDK4, and SMARCB1.

In embodiments, an increase in gene expression (e.g., the amount of mRNA expressed) may be about a 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about a 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more decrease in gene expression relative to a cell that has not been contacted with a perturbagen and/or relative to a cell that has been contacted with a no treatment control (including DMSO).

In various embodiments, an increase in gene expression (e.g., the amount of mRNA expressed) may be about a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater decrease in gene expression relative to a cell that has not been contacted with a perturbagen and/or relative to a cell that has been contacted with a no treatment control (including DMSO).

In embodiments, contacting the population of cells comprising a progenitor cell occurs in vitro or ex vivo.

In embodiments, contacting the population of cells comprising a progenitor cell occurs in vivo in a subject. In embodiments, the subject is a human. In embodiments, the human is an adult human.

In an aspect, the present disclosure provides a method for promoting the formation of a neutrophil, monocyte or immediate progenitor thereof. The method comprising a step of exposing a starting population of stem/progenitor cells comprising a non-lineage committed CD34+ cell to a perturbagen which promotes the transition of the starting population of stem/progenitor cells into a granulocyte monocyte progenitor, neutrophil progenitor, monocyte progenitor or other lineage-associated progenitor thereof. Embodiments associated with the above aspect are likewise relevant to the present aspect. In other words, each of the embodiments mentioned above for the above aspect may be revised/adapted to be applicable to the present aspect.

In an aspect, the present disclosure provides a method for promoting the formation of a neutrophil, monocyte or immediate progenitor thereof. The method comprising a step of exposing a starting population of stem/progenitor cells comprising a non-lineage committed CD34+ cell to a perturbation having a perturbation signature that promotes the transition of the starting population of stem/progenitor cells into a granulocyte monocyte progenitor, neutrophil progenitor, monocyte progenitor or other lineage-associated progenitor thereof. In this aspect, the perturbation signature comprises increased expression and/or activity of one or more of genes selected from Table 1 and/or a decreased expression and/or activity in the non-lineage committed CD34+ cell of one or more genes selected from Table 2. Embodiments associated with the above aspects are likewise relevant to the present aspect. In other words, each of the embodiments mentioned above for the above aspects may be revised/adapted to be applicable to the present aspect.

In another aspect, the present disclosure provides a method of increasing a quantity of neutrophils, monocytes or immediate progenitors thereof. The method comprising a step of exposing a starting population of stem/progenitor cells comprising a non-lineage committed CD34+ cell to a pharmaceutical composition that promotes the formation of lineage specific progenitor population selected from granulocyte monocyte progenitor, neutrophil progenitor, monocyte progenitor, or other lineage-associated progenitors. The pharmaceutical composition promotes the transition of a primitive stem/progenitor population into the lineage specific progenitor population that has the capacity to differentiate into neutrophils, monocytes or immediate progenitors thereof. In this aspect, the pharmaceutical composition comprises at least one perturbagen selected from Table 3, or a variant thereof. Embodiments associated with the above aspects are likewise relevant to the present aspect. In other words, each of the embodiments mentioned above for the above aspects may be revised/adapted to be applicable to the present aspect.

In yet another aspect, the present disclosure provides a perturbagen for use in any herein disclosed method.

In a further aspect, the present disclosure provides a pharmaceutical composition comprising perturbagen for use in any herein disclosed method.

### Methods and Perturbagens for Treating a Disease or Disorder

The ability of a perturbagen to specifically promote neutrophil and/or monocyte lineages (including the GMP lineage) would be valuable in designing a therapeutic composition. As examples, for a disease characterized by a reduced number of neutrophils, a therapeutic composition comprising a perturbagen that increases the number of neutrophils could be beneficial and/or a disease (including the same disease) that would benefit from increased numbers of monocytes could be treated by a therapeutic composition comprising a perturbagen that increases the number of monocytes.

An aspect of the present disclosure is a method for treating a disease or disorder characterized by an abnormal ratio of neutrophils to monocytes. The method comprising a step of: (a) administering to a patient in need thereof a therapeutically effective amount of at least one perturbagen selected from Table 3, or a variant thereof, in which the at least one perturbagen is capable of changing a gene signature in a progenitor cell; or (b) administering to a patient in need thereof a cell, the cell having been contacted with at least one perturbagen selected from Table 3, or a variant thereof, in which the at least one perturbagen is capable of changing a gene signature in a progenitor cell.

An aspect of the present disclosure is a method for treating a disease or disorder characterized by an abnormal ratio of neutrophils to monocytes. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

In embodiments, the abnormal ratio comprises an increased number of neutrophils and/or a decreased number of monocytes.

In embodiments, the abnormal ratio comprises a decreased number of neutrophils and/or an increased number of monocytes.

In embodiments, the abnormal ratio comprises a decreased number of neutrophils and a decreased number of monocytes.

In embodiments, the administering is directed to the bone marrow of the patient. In embodiments, the administering is via intraosseous injection or intraosseous infusion.

In embodiments, the administering the cell is via intravenous injection or intravenous infusion.

In embodiments, the administering is simultaneously or sequentially to one or more mobilization agents.

In embodiments, the disease or disorder characterized by an abnormal ratio of neutrophils to monocytes is selected from leukopenia, leukocytosis, agranulocytosis, granulocytosis, monocytopenia, monocytosis, neutropenia, and neutrophilia, leukemia (e.g., Chronic myeloid leukemia (CML), Chronic myelomonocytic leukemia (CMML), and Acute myeloblastic leukemia (AML), including AML-M5), cancer induced neutropenia, drug induced neutropenia, infection induce neutropenia, lupus (and other autoimmune disorders showing neutropenia), myelofibrosis, Chronic idiopathic neutropenia (CIN), and/or Autoimmune idiopathic neutropenia (AlN).

In embodiments, the disease or disorder characterized by an abnormal ratio of neutrophils to monocytes is related to an autoimmune disease, a bone-marrow transplant, a cancer, a genetic disease, a bacterial infection, a viral infection, inflammatory bowel disease, tissue necrosis (e.g., myocardial infarction and burns), a previous or concurrent anti-cancer treatment, and/or, a previous or concurrent immune suppressive treatment.

Another aspect of the present disclosure is a method for treating a disease or disorder characterized by an abnormal ratio of neutrophils and/or monocytes to progenitor cells. The method comprising a step of: (a) administering to a patient in need thereof at least one perturbagen selected from Table 3, or a variant thereof; or (b) administering to a patient in need thereof a cell, the cell having been contacted with at least one perturbagen selected from Table 3, or a variant thereof.

Another aspect of the present disclosure is a method for treating a disease or disorder characterized by an abnormal ratio of neutrophils and/or monocytes to progenitor cells. The method comprising a step of: (a) administering to a patient in need thereof at least one perturbagen selected from Table 3, or a variant thereof, in which the at least one perturbagen is capable of changing a gene signature in a progenitor cell; or (b) administering to a patient in need thereof a cell, the cell having been contacted with at least one perturbagen selected from Table 3, or a variant thereof, in which the at least one perturbagen is capable of changing a gene signature in a progenitor cell.

In embodiments, the abnormal ratio comprises a decreased number of neutrophils and/or a decreased number of monocytes and/or an increased number of progenitor cells. In embodiments, the abnormal ratio comprises a decreased number of neutrophils and an increased number of progenitor cells. In embodiments, the abnormal ratio comprises a decreased number of monocytes and an increased number of progenitor cells. In embodiments, the abnormal ratio comprises a decreased number of neutrophils and a decreased number of monocytes. In embodiments, the abnormal ratio comprises an increased number of progenitor cells.

In embodiments, the administering is directed to the bone marrow of the patient. In embodiments, the administering is via intraosseous injection or intraosseous infusion.

In embodiments, the administering the cell is via intravenous injection or intravenous infusion. In embodiments, the administering is simultaneously or sequentially to one or more mobilization agents. In embodiments, the disease or disorder characterized by an abnormal ratio of neutrophils and/or monocytes to progenitor cells is selected from leukopenia, leukocytosis, agranulocytosis, granulocytosis, monocytopenia, monocytosis, neutropenia, and neutrophilia, leukemia (e.g., Chronic myeloid leukemia (CML), Chronic myelomonocytic leukemia (CMML), and Acute myeloblastic leukemia (AML), including AML-M5), cancer induced neutropenia, drug induced neutropenia, infection induce neutropenia, lupus (and other autoimmune disorders showing neutropenia), myelofibrosis, Chronic idiopathic neutropenia (CIN), and/or Autoimmune idiopathic neutropenia (AlN).

In embodiments, the disease or disorder characterized by an abnormal ratio of neutrophils and/or monocytes to progenitor cells is related to an autoimmune disease, a bone-marrow transplant, a cancer, a genetic disease, a bacterial infection, a viral infection, inflammatory bowel disease, tissue necrosis (e.g., myocardial infarction and burns), a previous or concurrent anti-cancer treatment, and/or, a previous or concurrent immune suppressive treatment.

In embodiments, an abnormal ratio (e.g., of neutrophils to monocytes, neutrophils to progenitors, and monocytes to progenitors) is relative to the normal, healthy state. As examples, diseases that are characterized by an increased amount/number of neutrophils (e.g., neutrophilia), diseases that are characterized by a reduced amount/number of neutrophils (e.g., neutropenia), diseases that are characterized by a reduced amount/number of monocytes (e.g., monocytopenia), and diseases that are characterized by an increased amount/number of monocytes (e.g., monocytosis). Normal ratios of cell types in the blood is well known in the art.

Yet another aspect of the present disclosure is a method for treating monocytopenia. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

In an aspect, the present disclosure provides a method for treating monocytosis. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

In another aspect, the present disclosure provides a method for treating neutropenia. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

In yet another aspect, the present disclosure provides a method for treating neutrophilia. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

In a further aspect, the present disclosure provides a method for treating agranulocytosis. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof. In embodiments, the agranulocytosis is monocytopenia. In embodiments, the agranulocytosis is neutropenia.

An aspect of the present disclosure is a method for treating granulocytosis. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof. In embodiments, the agranulocytosis is monocytosis. In embodiments, the agranulocytosis is neutrophilia.

In embodiments, the at least one perturbagen is capable of changing a gene signature in a progenitor cell.

Another aspect of the present disclosure is a method of treating a disorder selected from drug-induced neutropenia, a side effect of bone marrow transplantation, or MonoMAC syndrome. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof. In embodiments, the drug-induced neutropenia in associated with a prior or concurrent chemotherapy and/or radiotherapy treatment. In embodiments, the drug-induced neutropenia is a consequence treatment with a cytotoxic chemotherapy, clozapine, dapsone, methimazole, penicillin, rituximab, and/or procainamide.

In embodiments, the therapeutically effective amount of the at least one perturbagen provides a change in a gene signature in a cell. In this embodiment, the change in the gene signature comprises an increase in expression and/or activity in the cell of one or more genes selected from Table 1 and/or a decrease in expression and/or activity in the cell of one or more genes selected from Table 2.

In embodiments, the therapeutically effective amount of the at least one perturbagen provides an increase in the number of neutrophils and/or the number of monocytes in a sample from the human subject relative to the number of neutrophils and/or the number of monocytes in a sample from the human subject prior to administering the at least one perturbagen.

In embodiments, the therapeutically effective amount of the at least one perturbagen provides an increase in the ratio of the number of neutrophils to the number of monocytes in a sample from the human subject relative to the ratio in a sample from the human subject prior to administering the at least one perturbagen.

In embodiments, the therapeutically effective amount of the at least one perturbagen provides an increase in the ratio of the number of monocytes to the number of neutrophils in a sample from the human subject relative to the ratio in a sample from the human subject prior to administering the at least one perturbagen.

In embodiments, the therapeutically effective amount of the at least one perturbagen provides an increase in the ratio of the number of neutrophils and/or the number of monocytes to the number of progenitor cells in a sample from the human subject relative to the ratio in a sample from the human subject prior to administering the at least one perturbagen.

In embodiments, the administering the therapeutically effective amount of the at least one perturbagen is directed to the bone marrow of the patient. In embodiments, the administering is via intraosseous injection or intraosseous infusion.

In embodiments, the administering the cell is via intravenous injection or intravenous infusion. In embodiments, the administering is simultaneously or sequentially to one or more mobilization agents. In embodiments, the administering occurs about once per day for one or more days.

In embodiments, the administering occurs more than once per day for one or more days.

In embodiments, the administering occurs at most once per day for one or more days.

In embodiments, the administering occurs substantially continuously per administration period.

In embodiments, the patient was selected by steps of, at least, obtaining from a subject having a disease or disorder a sample of cells comprising a non-lineage committed CD34+ cell and contacting the sample of cells with least one perturbagen selected from Table 3, or a variant thereof. In this embodiment, the at least one perturbagen alters a gene signature in the sample of cells.

In embodiments, the patient was selected by steps of, at least, obtaining from a subject having a disease or disorder a sample of cells comprising a non-lineage committed CD34+ cell and contacting the sample of cells with at least one perturbagen capable of altering a gene signature in a non-lineage committed CD34+ cell. In this embodiment, the at least one perturbagen increases in the sample of cells the expression and/or activity of one or more genes selected from Table 1 and/or decreases in the sample of cells the expression and/or activity of one or more genes selected from Table 2.

In embodiments, the patient was selected by steps of, at least, obtaining from a subject having a disease or disorder a sample of cells comprising a non-lineage committed CD34+ cell and contacting the sample of cells with at least one perturbagen selected from Table 3, or a variant thereof. In this embodiment, the at least one perturbagen increases in the sample of cells the expression and/or activity of one or more genes selected from Table 1 and/or decreases in the sample of cells the expression and/or activity of one or more genes selected from Table 2.

### Administration, Dosing, and Treatment Regimens

As examples, administration results in the delivery of one or more perturbagens disclosed herein into the bloodstream (via enteral or parenteral administration), or alternatively, the one or more perturbagens is administered directly to the site of hematopoietic cell proliferation and/or maturation, i.e., in the bone marrow.

Delivery of one or more perturbagens disclosed herein to the bone marrow may be via intravenous injection or intravenous infusion or via intraosseous injection or intraosseous infusion. Devices and apparatuses for performing these delivery methods are well known in the art.

Delivery of one or more perturbagens disclosed herein into the bloodstream via intravenous injection or intravenous infusion may follow or be contemporaneous with stem cell mobilization. In stem cell mobilization, certain drugs are used to cause the movement of stem cells from the bone marrow into the bloodstream. Once in the bloodstream, the stem cells are contacted with the one or more perturbagens and are able to alter a gene signature in and/or the state of a progenitor cell, for example. Drugs and methods relevant to stem cell mobilization are well known in the art; see, e.g., Mohammadi et al, “Optimizing Stem Cells Mobilization Strategies to Ameliorate Patient Outcomes: A Review of Guide-lines and Recommendations.” Int J Hematol Oncol Stem Cell Res. 2017 Jan. 1; 11 (1): 78-88; Hopman and DiPersio “Advances in Stem Cell Mobilization.” Blood Review, 2014, 28 (1): 31-40; and Kim “Hematopoietic stem cell mobilization: current status and future perspective.” Blood Res. 2017 June; 52 (2): 79-81. The contents of each of which is incorporated herein by reference in its entirety.

Dosage forms suitable for parenteral administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any perturbagen disclosed herein as well as the dosing schedule can depend on various parameters and factors, including, but not limited to, the specific perturbagen, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In another embodiment, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A perturbagen disclosed herein can be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled—or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled—or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, e.g., the bone marrow, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

The dosage regimen utilizing any perturbagen disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed. Any perturbagen disclosed herein can be administered in a single daily dose, or the total daily dosage can be administered in divided doses of two, three or four times daily. Furthermore, any perturbagen disclosed herein can be administered continuously rather than intermittently throughout the dosage regimen.

Pharmaceutical compositions and Formulations

Aspects of the present disclosure include a pharmaceutical composition comprising a therapeutically effective amount of one or more perturbagens, as disclosed herein.

The perturbagens disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. In embodiments, the compositions disclosed herein are in the form of a pharmaceutically acceptable salt.

Further, any perturbagen disclosed herein can be administered to a subject as a component of a composition, e.g., pharmaceutical composition that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any perturbagen disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In embodiments, the compositions, e.g., pharmaceutical compositions, disclosed herein are suspended in a saline buffer (including, without limitation TBS, PBS, and the like).

The present disclosure includes the disclosed perturbagens in various formulations of pharmaceutical compositions. Any perturbagens disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use.

Where necessary, the pharmaceutical compositions comprising the perturbagens can also include a solubilizing agent. Also, the agents can be delivered with a suitable vehicle or delivery device as known in the art.

Combination therapies, comprising more than one perturbagen, can be co-delivered in a single delivery vehicle or delivery device.

Compositions for administration can optionally include a local anesthetic such as, for example, lignocaine to lessen pain at the site of the injection.

The pharmaceutical compositions comprising the perturbagens of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any perturbagens disclosed herein is formulated in accordance with routine procedures as a pharmaceutical composition adapted for a mode of administration disclosed herein.

### Other Aspects of the Present Disclosure

Embodiments associated with any of the above-disclosed aspects are likewise relevant to the below-mentioned aspects. In other words, each of the embodiments mentioned above for the above aspects may be revised/adapted to be applicable to the below aspects.

Yet another aspect of the present disclosure is a use of the perturbagen of Table 3, or a variant thereof in the manufacture of a medicament for treating a disease or disorder characterized by an abnormal ratio of neutrophils to monocytes.

In an aspect, the present disclosure provides a use of the perturbagen of Table 3, or a variant thereof in the manufacture of a medicament for treating a disease or disorder characterized by an abnormal ratio of neutrophils and/or monocytes to progenitor cells.

An aspect of the present disclosure is a method for selecting a patient for treatment. The method comprising steps of obtaining from a subject having a disease or disorder a sample of cells comprising a non-lineage committed CD34+ cell and contacting the sample of cells with least one perturbagen selected from Table 3, or a variant thereof. In this aspect, when the at least one perturbagen increases the number of monocytes and/or neutrophils or a progenitor thereof in a culture from the sample of cells, the subject is selected as a patient.

An aspect of the present disclosure is a method for selecting a patient for treatment. The method comprising steps of obtaining from a subject having a disease or disorder a sample of cells comprising a non-lineage committed CD34+ cell and contacting the sample of cells with least one perturbagen selected from Table 3, or a variant thereof. In this aspect, when the at least one perturbagen alters a gene signature in the sample of cells, the subject is selected as a patient.

Another aspect of the present disclosure is a method for selecting a patient for treatment. The method comprising steps of obtaining from a subject having a disease or disorder a sample of cells comprising a non-lineage committed CD34+ cell and contacting the sample of cells with at least one perturbagen capable of altering a gene signature in a non-lineage committed CD34+ cell. In this aspect, when the at least one perturbagen increases in the sample of cells the expression and/or activity of one or more genes selected from Table 1 and/or decreases in the sample of cells the expression and/or activity of one or more genes selected from Table 2, the subject is selected as a patient.

Yet another aspect of the present disclosure is a method for selecting a patient for treatment. The method comprising steps of obtaining from a subject having a disease or disorder a sample of cells comprising a non-lineage committed CD34+ cell and contacting the sample of cells with at least one perturbagen selected from Table 3, or a variant thereof. In this aspect, when the at least one perturbagen increases in the sample of cells the expression and/or activity of one or more genes selected from Table 1 and/or decreases in the sample of cells the expression and/or activity of one or more genes selected from Table 2, the subject is selected as a patient.

In another aspect, the present disclosure provides a method of identifying a perturbagen for promoting the transition of a starting population of progenitor cells into neutrophils, monocytes or immediate progenitors thereof. The method comprising steps of: exposing the starting population of progenitor cells to a candidate perturbagen; determining a change in cell state of cells in the population of progenitor cells into neutrophils, monocytes or immediate progenitors thereof following exposure of the population of cells to the candidate perturbagen, wherein the candidate perturbagen capable changing the cell state of the cells in the population progenitor cells is identified as a perturbagen for promoting the transition of a starting population of progenitor cells into neutrophils, monocytes or immediate progenitors thereof.

In another aspect, the present disclosure provides a method for making a therapeutic agent for a disease or disorder selected from leukopenia, leukocytosis, agranulocytosis, granulocytosis, monocytopenia, monocytosis, neutropenia, and neutrophilia, leukemia, cancer induced neutropenia, drug induced neutropenia, infection induce neutropenia, lupus (and other autoimmune disorders showing neutropenia), myelofibrosis, Chronic idiopathic neutropenia (CIN), and Autoimmune idiopathic neutropenia (AlN). The method comprises steps of: (a) identifying a perturbagen capable of acting as a therapeutic agent for therapy; and (b) formulating the therapeutic agent for the treatment of the disease or disorder. In this aspect, identifying a perturbagen for promoting the transition of a starting population of progenitor cells into neutrophils, monocytes or immediate progenitors thereof. The method comprising steps of: exposing the starting population of progenitor cells to a candidate perturbagen; determining a change in cell state of cells in the population of progenitor cells into neutrophils, monocytes or immediate progenitors thereof following exposure of the population of cells to the candidate perturbagen, wherein the candidate perturbagen capable changing the cell state of the cells in the population progenitor cells is identified as a perturbagen for promoting the transition of a starting population of progenitor cells into neutrophils, monocytes or immediate progenitors thereof.

In another aspect, the present disclosure provides a method of identifying a candidate perturbation for promoting the transition of a starting population of progenitor cells into neutrophils, monocytes or immediate progenitors thereof. The method comprising steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of cells in the population of progenitor cells into neutrophils, monocytes or immediate progenitors thereof following exposure of the population of cells to the perturbation; and identifying the perturbation as a candidate perturbation for promoting the transition of a population of progenitor cells into neutrophils, monocytes or immediate progenitors thereof based on the perturbation signature. In this aspect, the perturbation signature is an increase in expression and/or activity in the progenitor cell of one or more genes selected from Table 1, and/or a decrease in expression and/or activity in the progenitor cell of one or more genes selected from Table 2.

In another aspect, the present disclosure provides a method for making a therapeutic agent for a disease or disorder selected from leukopenia, leukocytosis, agranulocytosis, granulocytosis, monocytopenia, monocytosis, neutropenia, and neutrophilia, leukemia, cancer induced neutropenia, drug induced neutropenia, infection induce neutropenia, lupus (and other autoimmune disorders showing neutropenia), myelofibrosis, Chronic idiopathic neutropenia (CIN), and Autoimmune idiopathic neutropenia (AlN). The method comprises steps of: (a) identifying a therapeutic agent for therapy; and (b) formulating the therapeutic agent for the treatment of the disease or disorder. In this aspect, identifying a therapeutic agent for therapy comprises steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell fate of the population of the population of progenitor cells into neutrophils, monocytes or immediate progenitors thereof following exposure of the population of cells to the perturbation; and identifying the perturbation as a candidate perturbation for promoting the transition of a population of progenitor cells into neutrophils, monocytes or immediate progenitors thereof based on the perturbation signature. Further, in this aspect, the perturbation signature is an increase in expression and/or activity in the progenitor cell of one or more genes selected from Table 1, and/or a decrease in expression and/or activity in the progenitor cell of one or more genes selected from Table 2.

In a further aspect, the present disclosure provides a method for obtaining a gene signature that defines genes relevant to cell fate towards the granulocyte monocyte progenitor (GMP) lineage or within the GMP lineage.

An aspect of the present disclosure is a method for obtaining a gene signature capable of directing a change in cell fate towards the granulocyte monocyte progenitor (GMP) lineage or within the GMP lineage.

Another aspect of the present disclosure is a use of gene signature to identify a perturbagen capable of directing a change in cell fate towards the granulocyte monocyte progenitor (GMP) lineage or within the GMP lineage. Yet another aspect of the present disclosure is a perturbagen capable of causing a change in a gene signature.

In an aspect, the present disclosure provides a perturbagen capable of causing a change in cell fate.

In another aspect, the present disclosure provides a perturbagen capable of causing a change in a gene signature and a change in cell fate.

In yet another aspect, the present disclosure provides a pharmaceutical composition comprising any herein disclosed perturbagen.

In a further aspect, the present disclosure provides a unit dosage form comprising an effective amount of the pharmaceutical composition comprising any herein disclosed perturbagen.

The instant disclosure also provides certain embodiments as follows:
Embodiment 1. A method for directing a change in cell state of a progenitor cell comprising: contacting a population of cells comprising a progenitor cell with at least one perturbagen selected from Table 3, or a variant thereof, wherein the at least one perturbagen is capable of altering a gene signature in the progenitor cell; and wherein the progenitor cell is a non-lineage committed CD34+ cell.
Embodiment 2. A method for directing a change in cell state of a progenitor cell, comprising: contacting a population of cells comprising a progenitor cell with at least one perturbagen capable of altering a gene signature in the progenitor cell, wherein altering the gene signature comprises an increase in expression and/or activity in the progenitor cell of one or more genes selected from Table 1 and/or a decrease in expression and/or activity in the progenitor cell of one or more genes selected from Table 2 and wherein the progenitor cell is a non-lineage committed CD34+ cell.
Embodiment 3. A method for directing a change in cell state of a progenitor cell, comprising: contacting a population of cells comprising a progenitor cell with at least one perturbagen selected from Table 3, or a variant thereof, and capable of altering a gene signature in the progenitor cell, wherein altering the gene signature comprises an increase in expression and/or activity in the progenitor cell of one or more genes selected from Table 1 and/or a decrease in expression and/or activity in the progenitor cell of one or more genes selected from Table 2 and wherein the progenitor cell is a non-lineage committed CD34+ cell.
Embodiment 4. The method of any one of Embodiments 1 to 3, wherein the change in cell state provides an increase in the number of neutrophils and/or the number of monocytes.
Embodiment 5. The method of Embodiment 4, wherein the increase in the number of neutrophils and/or the number of monocytes is relative to the number of neutrophils and/or the number of monocytes obtained from a population of progenitor cells that is not contacted with the at least one perturbagen.
Embodiment 6. The method of Embodiment 4, wherein the increase in the number of neutrophils and/or the number of monocytes is relative to the population of progenitor cells prior to contacting with the at least one perturbagen.
Embodiment 7. The method of Embodiment 5 or Embodiment 6, wherein the change in cell state provides an increase in the number of neutrophils.
Embodiment 8. The method of Embodiment 7, wherein the change in cell state provides an increase in the number of monocytes.
Embodiment 9. The method of Embodiment 7, wherein the change in cell state does not provide a substantial increase in the number of monocytes and/or provides a decrease in the number of monocytes.
Embodiment 10. The method of any one of Embodiments 7 to 9, wherein the ratio of the number of neutrophils to the number of monocytes is increased relative to the ratio obtained from a population of progenitor cells that is not contacted with the at least one perturbagen.
Embodiment 11. The method of any one of Embodiments 7 to 9, wherein the ratio of the number of neutrophils to the number of monocytes is increased relative to the ratio in the population of progenitor cells prior to contacting with the at least one perturbagen.
Embodiment 12. The method of any one of Embodiments 4 to 11, wherein the change in cell state provides an increase in the number of monocytes.
Embodiment 13. The method of Embodiment 12, wherein the change in cell state provides an increase in the number of neutrophils.
Embodiment 14. The method of Embodiment 12, wherein the change in cell state does not provide a substantial increase in the number of neutrophils and/or provides a decrease in the number of neutrophils.
Embodiment 15. The method of any one of Embodiments 12 to 14, wherein the ratio of the number of monocytes to the number of neutrophils is increased relative to the ratio obtained from a population of progenitor cells that is not contacted with the at least one perturbagen.
Embodiment 16. The method of any one of Embodiments 12 to 14, wherein the ratio of the number of monocytes to the number of neutrophils is increased relative to the ratio in the population of progenitor cells prior to contacting with the at least one perturbagen.
Embodiment 17. The method of Embodiment 4, wherein the ratio of the number of neutrophils to the number of progenitor cells is increased relative to the ratio obtained from a population of progenitor cells that is not contacted with the at least one perturbagen.
Embodiment 18. The method of Embodiment 4, wherein the ratio of the number of neutrophils to the number of progenitor cells is increased relative to the ratio in the population of progenitor cells prior to contacting with the at least one perturbagen.
Embodiment 19. The method of Embodiment 4, wherein the ratio of the number of monocytes to the number of progenitor cells is increased relative to the ratio obtained from a population of progenitor cells that is not contacted with the at least one perturbagen.
Embodiment 20. The method of Embodiment 4, wherein the ratio of the number of monocytes to the number of progenitor cells is increased relative to the ratio in the population of progenitor cells prior to contacting with the at least one perturbagen.
Embodiment 21. The method of any one of Embodiments 4 to 20, wherein the increase in the number of neutrophils and/or the number of monocytes is due in part to increased cell proliferation of the neutrophils and/or the monocytes.
Embodiment 22. The method of any one of Embodiments 4 to 21, wherein the increase in the number of neutrophils and/or the number of monocytes is due in part to an increased lifespan of the neutrophils and/or the monocytes.
Embodiment 23. The method of any one of Embodiments 4 to 22, wherein the increase in the number of neutrophils and/or the number of monocytes is due in part to reduced cell death among the neutrophils and/or the monocytes.
Embodiment 24. The method of any one of Embodiments 4 to 23, wherein the increase in the number of neutrophils and/or the number of monocytes is due in part to a change of cell state from progenitor cells into the neutrophil and/or monocyte lineage.
Embodiment 25. The method of any one of Embodiments 21 to 24, wherein the number of progenitor cells is decreased.
Embodiment 26. The method of Embodiment 25, wherein the decrease in the number of progenitor cells is due in part to decreased cell proliferation of the progenitor cells.
Embodiment 27. The method of Embodiment 25 or Embodiment 26, wherein the decrease in the number of progenitor cells is due in part to a decreased lifespan of the progenitor cells.
Embodiment 28. The method of any one of Embodiments 25 to 27, wherein the decrease in the number of progenitor cells is due in part to increased cell death among the progenitor cells.
Embodiment 29. The method of any one of Embodiments 25 to 28, wherein the decrease in the number of progenitor cells is relative to the number of progenitor cells in a population of progenitor cells that is not contacted with the at least one perturbagen.
Embodiment 30. The method of any one of Embodiments 25 to 29, wherein the decrease in the number of progenitor cells is relative to the number of progenitor cells in the population prior to contacting with the at least one perturbagen.
Embodiment 31. The method of any one of Embodiments Embodiment 25 to 30, wherein the decrease in the number of progenitor cells is due to a change of cell state from a progenitor cell into the neutrophil lineage and/or monocyte lineage.
Embodiment 32. The method of any one of Embodiments 21 to 24, wherein the number of progenitor cells is increased.
Embodiment 33. The method of Embodiment 32, wherein the increase in the number of progenitor cells is due in part to increased cell proliferation of the progenitor cells.
Embodiment 34. The method of Embodiment 32 or Embodiment 33, wherein the increase in the number of progenitor cells is due in part to an increased lifespan of the progenitor cells.
Embodiment 35. The method of any one of Embodiments 32 to 34, wherein the increase in the number of progenitor cells is due in part to decreased cell death among the progenitor cells.
Embodiment 36. The method of any one of Embodiments 32 to 35, wherein the increase in the number of progenitor cells is relative to the number of progenitor cells in a population of progenitor cells that is not contacted with the at least one perturbagen.
Embodiment 37. The method of any one of Embodiments 32 to 35, wherein the increase in the number of progenitor cells is relative to the number of progenitor cells in the population prior to contacting with the at least one perturbagen.
Embodiment 38. The method of any one of Embodiments 21 to 24, wherein the number of CD66b+ neutrophil progenitors, CD64+monocytic progenitors, neutrophilic promyelocytes, neutrophilic myelocytes, neutrophilic metamyelocytes, and/or neutrophilic band cells is increased and/or the number of CD34+CD38+/− cells are increased about two days after contacting the population of cells comprising a CD34+ cell with the at least one perturbagen and/or the number of CD34-CD38+/− cells are increased about five days after contacting the population of cells comprising a CD34+ cell with the at least one perturbagen.
Embodiment 39. The method of Embodiment 38, wherein the ratio of the number of CD66b+ neutrophil progenitors, CD64+monocytic progenitors, neutrophilic promyelocytes, neutrophilic myelocytes, neutrophilic metamyelocytes, neutrophilic band cells, CD34+CD38+/− cells, and/or CD34-CD38+/− cells to the number of progenitor cells is increased relative to the ratio obtained from a population of progenitor cells that is not contacted with the at least one perturbagen.
Embodiment 40. The method of Embodiment 38, wherein the ratio of the number of CD66b+ neutrophil progenitors, CD64+monocytic progenitors, neutrophilic promyelocytes, neutrophilic myelocytes, neutrophilic cells is increased relative to the ratio in the population of progenitor cells prior to contacting with the at least one perturbagen.
Embodiment 41. The method of any one of Embodiments 21 to 24, wherein the number of monoblasts and/or promonocytes is increased.
Embodiment 42. The method of Embodiment 41, wherein the ratio of the number of monoblasts and/or promonocytes to the number of progenitor cells is increased relative to the ratio obtained from a population of progenitor cells that is not contacted with the at least one perturbagen.
Embodiment 43. The method of Embodiment 41, wherein the ratio of the number of monoblasts and/or promonocytes to the number of progenitor cells is increased relative to the ratio in the population of progenitor cells prior to contacting with the at least one perturbagen.
Embodiment 44. The method of any one of Embodiments 21 to 24, wherein the number of granulocyte-monocyte progenitor (GMP) cells is increased.
Embodiment 45. The method of Embodiment 44, wherein the ratio of the number of GMP cells to the number of progenitor cells for the GMP cell is increased relative to the ratio obtained from a population of progenitor cells that is not contacted with the at least one perturbagen.
Embodiment 46. The method of Embodiment 44, wherein the ratio of the number of GMP cells to the number of progenitor cells for the GMP cell is increased relative to the ratio in the population of progenitor cells prior to contacting with the at least one perturbagen.
Embodiment 47. The method of Embodiment 46, wherein the progenitor cell for the GMP cell is a common myeloid progenitor (CMP).
Embodiment 48. The method of Embodiment 44, wherein the number of monocyte-dendritic cell progenitor (MDP) cells is decreased.
Embodiment 49. The method of Embodiment 48, wherein the ratio of the number of GMP cells to the number of MDP cells is increased relative to the ratio obtained from a population of progenitor cells that is not contacted with the at least one perturbagen.
Embodiment 50. The method of Embodiment 48, wherein the ratio of the number of GMP cells to the number of MDP cells is increased relative to the ratio in the population of progenitor cells prior to contacting with the at least one perturbagen.
Embodiment 51. The method of any one of Embodiments 1 to 50, wherein the at least one perturbagen selected from Table 3, or a variant thereof, comprises at least 2, at least 3, at least 4, at least 5, at least 6, at least 7, at least 8, at least 9, at least 10, at least 11, or 12 perturbagens selected from Table 3, or variants thereof.
Embodiment 52. The method of Embodiment 51, wherein the at least one perturbagen promotes the monocyte lineage.
Embodiment 53. The method of Embodiment 51, wherein the at least one perturbagen promotes the neutrophil lineage.
Embodiment 54. The method of any one of Embodiments 1 to 53, wherein altering the gene signature comprises increased expression and/or increased activity in the progenitor cell of one or more genes selected from Table 1.
Embodiment 55. The method of Embodiment 54, wherein the one or more genes selected from Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, or 49 genes selected from Table 1.
Embodiment 56. The method of Embodiment 54 or Embodiment 55, wherein the one or more genes selected from Table 1 comprises at least one of MYB, NCOA4, CEBPD, CEBPA, TLE4, DACH1, SMARCA2, GFI1, MAZ, TLE1, ELL2, and ARID1A.
Embodiment 57. The method of Embodiment 56, wherein the one or more genes selected from Table 1 comprises at least one of MYB, NCOA4, CEBPD, CEBPA, and TLE4.
Embodiment 58. The method of any one of Embodiments 1 to 57, wherein altering the gene signature comprises decreased expression and/or decreased activity in the progenitor cell of one or more genes selected from Table 2
Embodiment 59. The method of Embodiment 58, wherein the one or more genes selected from Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, or 72 genes selected from Table 2.
Embodiment 60. The method of Embodiment 58 or Embodiment 59, wherein the one or more genes selected from Table 2 comprises at least one of HMGA1, SSBP4, LSM4, CDK4, SMARCB1, AES, GTF3A, RAB8A, RNPS1, THRAP3, and SNRPB.
Embodiment 61. The method of Embodiment 60, wherein the one or more genes selected from Table 2 comprises at least one of HMGA1, SSBP4, LSM4, CDK4, and SMARCB1.
Embodiment 62. The method of any one of Embodiments 1 to 61, wherein contacting the population of progenitor cells occurs in vitro or ex vivo.
Embodiment 63. The method of any one of Embodiments 1 to 61, wherein contacting the population of progenitor cells occurs in vivo in a subject.
Embodiment 64. The method of Embodiment 63, wherein the subject is a human.
Embodiment 65. The method of Embodiment 64, wherein the human is an adult human.
Embodiment 66. A perturbagen for use in the method of any one of Embodiments 1 to 65.
Embodiment 67. A pharmaceutical composition comprising the perturbagen of Embodiment 66.
Embodiment 68. A method for promoting the formation of a neutrophil, a monocyte, or an immediate progenitor thereof, comprising: exposing a starting population of stem/progenitor cells comprising a non-lineage committed CD34+ cell to a perturbation having a perturbation signature that promotes the transition of the starting population of stem/progenitor cells into a granulocyte monocyte progenitor, neutrophil progenitor, monocyte progenitor or other lineage associated progenitor thereof, wherein the perturbation signature comprises increased expression and/or activity of one or more of genes selected from Table 1 and/or a decreased expression and/or activity in the non-lineage committed CD34+ cell of one or more genes selected from Table 2.
Embodiment 69. A method of increasing a quantity of neutrophils, monocytes, or immediate progenitors thereof, comprising: exposing a starting population of stem/progenitor cells comprising a non-lineage committed CD34+ cell to a pharmaceutical composition that promotes the formation of lineage specific progenitor population selected from granulocyte monocyte progenitor, neutrophil progenitor, monocyte progenitor, or other lineage-associated progenitors, the pharmaceutical composition promoting the transition of a primitive stem/progenitor population into the lineage specific progenitor population that has the capacity to differentiate into neutrophils, monocytes or immediate progenitors thereof, wherein the pharmaceutical composition comprises at least one perturbagen selected from Table 3, or a variant thereof.
Embodiment 70. A method for treating a disease or disorder characterized by an abnormal ratio of neutrophils to monocytes, comprising: (a) administering to a patient in need thereof a therapeutically effective amount of at least one perturbagen selected from Table 3, or a variant thereof, wherein the at least one perturbagen is capable of changing a gene signature in a progenitor cell or (b) administering to a patient in need thereof a cell, the cell having been contacted with at least one perturbagen selected from Table 3, or a variant thereof, wherein the at least one perturbagen is capable of changing a gene signature in a progenitor cell.
Embodiment 71. The method of Embodiment 70, wherein the abnormal ratio comprises an increased number of neutrophils and/or a decreased number of monocytes.
Embodiment 72. The method of Embodiment 70, wherein the abnormal ratio comprises a decreased number of neutrophils and/or an increased number of monocytes.
Embodiment 73. The method of Embodiment 70, wherein the abnormal ratio comprises a decreased number of neutrophils and a decreased number of monocytes.
Embodiment 74. The method of any one of Embodiments 70 to 73, wherein the administering is directed to the bone marrow of the patient.
Embodiment 75. The method of Embodiment 74, wherein the administering is via intraosseous injection or intraosseous infusion.
Embodiment 76. The method of any one of Embodiments 70 to 73, wherein the administering the cell is via intravenous injection or intravenous infusion.
Embodiment 77. The method of any one of Embodiments 70 to 76, wherein the administering is simultaneously or sequentially to one or more mobilization agents.
Embodiment 78. The method of any one of Embodiments 70 to 77, wherein the disease or disorder characterized by an abnormal ratio of neutrophils to monocytes is selected from leukopenia, leukocytosis, agranulocytosis, granulocytosis, monocytopenia, monocytosis, neutropenia, and neutrophilia, leukemia (e.g., Chronic myeloid leukemia (CML), Chronic myelomonocytic leukemia (CMML), and Acute myeloblastic leukemia (AML), including AML-M5), cancer induced neutropenia, drug induced neutropenia, infection induce neutropenia, lupus (and other autoimmune disorders showing neutropenia), myelofibrosis, Chronic idiopathic neutropenia (CIN), and/or Autoimmune idiopathic neutropenia (AlN).
Embodiment 79. The method of any one of Embodiments 70 to 78, wherein the disease or disorder characterized by an abnormal ratio of neutrophils to monocytes is related to an autoimmune disease, a bone-marrow transplant, a cancer, a genetic disease, a bacterial infection, a viral infection, inflammatory bowel disease, tissue necrosis (e.g., myocardial infarction and burns), a previous or concurrent anti-cancer treatment, and/or, a previous or concurrent immune suppressive treatment.
Embodiment 80. A method for treating a disease or disorder characterized by an abnormal ratio of neutrophils and/or monocytes to progenitor cells, comprising: (a) administering to a patient in need thereof at least one perturbagen selected from Table 3, or a variant thereof, wherein the at least one perturbagen is capable of changing a gene signature in a progenitor cell or (b) administering to a patient in need thereof a cell, the cell having been contacted with at least one perturbagen selected from Table 3, or a variant thereof, wherein the at least one perturbagen is capable of changing a gene signature in a progenitor cell.
Embodiment 81. The method of Embodiment 80, wherein the abnormal ratio comprises a decreased number of neutrophils and/or a decreased number of monocytes and/or an increased number of progenitor cells.
Embodiment 82. The method of Embodiment 81, wherein the abnormal ratio comprises a decreased number of neutrophils and an increased number of progenitor cells.
Embodiment 83. The method of Embodiment 81, wherein the abnormal ratio comprises a decreased number of monocytes and an increased number of progenitor cells.
Embodiment 84. The method of Embodiment 81, wherein the abnormal ratio comprises a decreased number of neutrophils and a decreased number of monocytes.
Embodiment 85. The method of Embodiment 84, wherein the abnormal ratio comprises an increased number of progenitor cells.
Embodiment 86. The method of any one of Embodiments 80 to 85, wherein the administering is directed to the bone marrow of the patient.
Embodiment 87. The method of Embodiment 86, wherein the administering is via intraosseous injection or intraosseous infusion.
Embodiment 88. The method of any one of Embodiments 80 to 85, wherein the administering the cell is via intravenous injection or intravenous infusion.
Embodiment 89. The method of any one of Embodiments 80 to 88, wherein the administering is simultaneously or sequentially to one or more mobilization agents.
Embodiment 90. The method of any one of Embodiments 88 to 89, wherein the disease or disorder characterized by an abnormal ratio of neutrophils and/or monocytes to progenitor cells is selected from leukopenia, leukocytosis, agranulocytosis, granulocytosis, monocytopenia, monocytosis, neutropenia, and neutrophilia, leukemia (e.g., Chronic myeloid leukemia (CML), Chronic myelomonocytic leukemia (CMML), and Acute myeloblastic leukemia (AML), including AML-M5), cancer induced neutropenia, drug induced neutropenia, infection induce neutropenia, lupus (and other autoimmune disorders showing neutropenia), myelofibrosis, Chronic idiopathic neutropenia (CIN), and/or Autoimmune idiopathic neutropenia (AlN).
Embodiment 91. The method of any one of Embodiments 80 to 90, wherein the disease or disorder characterized by an abnormal ratio of neutrophils and/or monocytes to progenitor cells is related to an autoimmune disease, a bone-marrow transplant, a cancer, a genetic disease, a bacterial infection, a viral infection, inflammatory bowel disease, tissue necrosis (e.g., myocardial infarction and burns), a previous or concurrent anti-cancer treatment, and/or, a previous or concurrent immune suppressive treatment.
Embodiment 92. A method for treating monocytopenia, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 93. A method for treating monocytosis, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 94. A method for treating neutropenia, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 95. A method for treating neutrophilia, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 96. A method for treating agranulocytosis, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 97. The method of Embodiment 96, wherein the agranulocytosis is monocytopenia.
Embodiment 98. The method of Embodiment 96, wherein the agranulocytosis is neutropenia.
Embodiment 99. A method for treating granulocytosis, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 100. The method of Embodiment 99, wherein the agranulocytosis is monocytosis.
Embodiment 101. The method of Embodiment 99, wherein the agranulocytosis is neutrophilia.
Embodiment 102. The method of any one of Embodiments 92 to 101, wherein the at least one perturbagen is capable of changing a gene signature in a progenitor cell.
Embodiment 103. A method of treating a disorder selected from drug-induced neutropenia, a side-effect of bone marrow transplantation, or MonoMAC syndrome, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 104. The method of Embodiment 103, wherein the drug-induced neutropenia in associated with a prior or concurrent chemotherapy and/or radiotherapy treatment.
Embodiment 105. The method of any one of Embodiments 92 to 104, wherein the therapeutically effective amount of the at least one perturbagen provides a change in a gene signature in a cell, wherein the change in the gene signature comprises an increase in expression and/or activity in the cell of one or more genes selected from Table 1 and/or a decrease in expression and/or activity in the cell of one or more genes selected from Table 2.

### Embodiment

Embodiment 106. The method of any one of Embodiments 92 to 105, wherein the therapeutically effective amount of the at least one perturbagen provides an increase in the number of neutrophils and/or the number of monocytes in a sample from the human subject relative to the number of neutrophils and/or the number of monocytes in a sample from the human subject prior to administering the at least one perturbagen.
Embodiment 107. The method of any one of Embodiments 92 to 106, wherein the therapeutically effective amount of the at least one perturbagen provides an increase in the ratio of the number of neutrophils to the number of monocytes in a sample from the human subject relative to the ratio in a sample from the human subject prior to administering the at least one perturbagen.
Embodiment 108. The method of any one of Embodiments 92 to 107, wherein the therapeutically effective amount of the at least one perturbagen provides an increase in the ratio of the number of monocytes to the number of neutrophils in a sample from the human subject relative to the ratio in a sample from the human subject prior to administering the at least one perturbagen.
Embodiment 109. The method of any one of Embodiments 92 to 108, wherein the therapeutically effective amount of the at least one perturbagen provides an increase in the ratio of the number of neutrophils and/or the number of monocytes to the number of progenitor cells in a sample from the human subject relative to the ratio in a sample from the human subject prior to administering the at least one perturbagen.
Embodiment 110. The method of any one of Embodiments 92 to 109, wherein the administering the therapeutically effective amount of the at least one perturbagen is directed to the bone marrow of the patient.
Embodiment 111. The method of Embodiment 110, wherein the administering is via intraosseous injection or intraosseous infusion.
Embodiment 112. The method of any one of Embodiments 92 to 109, wherein the administering the cell is via intravenous injection or intravenous infusion.
Embodiment 113. The method of any one of Embodiments 92 to 109, wherein the administering is simultaneously or sequentially to one or more mobilization agents.
Embodiment 114. The method of any one of Embodiments 70 to 109, wherein the administering occurs about once per day for one or more days.
Embodiment 115. The method of any one of Embodiments 70 to 109, wherein the administering occurs more than once per day for one or more days.
Embodiment 116. The method of any one of Embodiments 70 to 109, wherein the administering occurs at most once per day for one or more days.
Embodiment 117. The method of any one of Embodiments 70 to 109, wherein the administering occurs substantially continuously per administration period.
Embodiment 118. The method of any one of Embodiments 70 to 117, wherein the patient was selected by steps comprising: obtaining from the patient having the disease or disorder a sample of cells comprising a non-lineage committed CD34+ cell; and contacting the sample of cells with least one perturbagen selected from Table 3, or a variant thereof, wherein the at least one perturbagen alters a gene signature in the sample of cells. Embodiment 119. The method of any one of Embodiments 70 to 117, wherein the patient was selected by steps comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein the at least one perturbagen increases in the sample of cells the expression and/or activity of one or more genes selected from Table 1 and/or decreases in the sample of cells the expression and/or activity of one or more genes selected from Table 2.
Embodiment 120. The method of any one of Embodiments 70 to 117, wherein the patient was selected by steps comprising: obtaining from a subject having the disease or disorder a sample of cells comprising a non-lineage committed CD34+ cell; and contacting the sample of cells with at least one perturbagen selected from Table 3, or a variant thereof; wherein the at least one perturbagen increases in the sample of cells the expression and/or activity of one or more genes selected from Table 1 and/or decreases in the sample of cells the expression and/or activity of one or more genes selected from Table 2.
Embodiment 121. A method for selecting the patient of any one of Embodiments 70 to 117 comprising: obtaining from a subject having the disease or disorder a sample of cells comprising a non-lineage committed CD34+ cell; and contacting the sample of cells with least one perturbagen selected from Table 3, or a variant thereof, wherein when the at least one perturbagen alters a gene signature in the sample of cells, the subject is selected as a patient. Embodiment 122. A method for selecting the patient of any one of Embodiments 70 to 117 comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein when the at least one perturbagen increases in the sample of cells the expression and/or activity of one or more genes selected from Table 1 and/or decreases in the sample of cells the expression and/or activity of one or more genes selected from Table 2, the subject is selected as a patient.
Embodiment 123. A method for selecting the patient of any one of Embodiments 70 to 117 comprising: obtaining from a subject having the disease or disorder a sample of cells comprising a non-lineage committed CD34+ cell; and contacting the sample of cells with at least one perturbagen selected from Table 3, or a variant thereof; wherein when the at least one perturbagen increases in the sample of cells the expression and/or activity of one or more genes selected from Table 1 and/or decreases in the sample of cells the expression and/or activity of one or more genes selected from Table 2, the subject is selected as a patient.
Embodiment 124. Use of the perturbagen of Table 3, or a variant thereof in the manufacture of a medicament for treating a disease or disorder characterized by an abnormal ratio of neutrophils to monocytes. Embodiment 125. Use of the perturbagen of Table 3, or a variant thereof in the manufacture of a medicament for treating a disease or disorder characterized by an abnormal ratio of neutrophils and/or monocytes to progenitor cells.
Embodiment 126. A method of identifying a candidate perturbation for promoting the transition of a starting population of progenitor cells into neutrophils, monocytes or immediate progenitors thereof, the method comprising: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into neutrophils, monocytes or immediate progenitors thereof following exposure of the population of cells to the perturbation; and identifying the perturbation as a candidate perturbation for promoting the transition of a population of progenitor cells into neutrophils, monocytes or immediate progenitors thereof based on the perturbation signature, wherein the perturbation signature is an increase in expression and/or activity in the progenitor cell of one or more genes selected from Table 1, and/or a decrease in expression and/or activity in the progenitor cell of one or more genes selected from Table 2.
Embodiment 127. A method for making a therapeutic agent for a disease or disorder selected from leukopenia, leukocytosis, agranulocytosis, granulocytosis, monocytopenia, monocytosis, neutropenia, and neutrophilia, leukemia, cancer induced neutropenia, drug induced neutropenia, infection induce neutropenia, lupus (and other autoimmune disorders showing neutropenia), myelofibrosis, Chronic idiopathic neutropenia (CIN), and Autoimmune idiopathic neutropenia (AlN), comprising: (a) identifying a therapeutic agent for therapy according to the method of Embodiment 126 and (b) formulating the therapeutic agent for the treatment of the disease or disorder.
Embodiment 2001. A method for directing a change in cell state of a progenitor cell comprising: contacting a population of cells comprising a progenitor cell with at least one perturbagen selected from Table 3, or a variant thereof, wherein the at least one perturbagen is capable of directing a change in cell state of a non-lineage committed CD34+progenitor cell and wherein the progenitor cell is a non-lineage committed CD34+ cell.
Embodiment 2002. The method of Embodiment 2001, wherein the change in cell state provides an increase in the number of neutrophils.
Embodiment 2003. The method of Embodiment 2001, wherein the change in cell state provides an increase in the number of monocytes.
Embodiment 2004. The method of any one of Embodiments 2001 to 2003, wherein the change in cell state provides an increase in the number of neutrophils and the number of monocytes.
Embodiment 2005. The method of Embodiment 2004, wherein the increase in the number of neutrophils and/or the number of monocytes is relative to the number of neutrophils and/or the number of monocytes obtained from a population of progenitor cells that is not contacted with the at least one perturbagen.
Embodiment 2006. The method of Embodiment 2004, wherein the increase in the number of neutrophils and/or the number of monocytes is relative to the population of progenitor cells prior to contacting with the at least one perturbagen.
Embodiment 2007. The method of Embodiment 2005 or Embodiment 2006, wherein the change in cell state provides an increase in the number of neutrophils.
Embodiment 2008. The method of Embodiment 2007, wherein the change in cell state provides an increase in the number of monocytes.
Embodiment 2009. The method of Embodiment 2007, wherein the change in cell state does not provide a substantial increase in the number of monocytes and/or provides a decrease in the number of monocytes.
Embodiment 2010. The method of any one of Embodiments 2007 to 2009, wherein the ratio of the number of neutrophils to the number of monocytes is increased relative to the ratio obtained from a population of progenitor cells that is not contacted with the at least one perturbagen.
Embodiment 2011. The method of any one of Embodiments 2007 to 2009, wherein the ratio of the number of neutrophils to the number of monocytes is increased relative to the ratio in the population of progenitor cells prior to contacting with the at least one perturbagen.
Embodiment 2012. The method of any one of Embodiments 2004 to 2011, wherein the change in cell state provides an increase in the number of monocytes.
Embodiment 2013. The method of Embodiment 2012, wherein the change in cell state provides an increase in the number of neutrophils.
Embodiment 2014. The method of Embodiment 2012, wherein the change in cell state does not provide a substantial increase in the number of neutrophils and/or provides a decrease in the number of neutrophils.
Embodiment 2015. The method of any one of Embodiments 2012 to 2014, wherein the ratio of the number of monocytes to the number of neutrophils is increased relative to the ratio obtained from a population of progenitor cells that is not contacted with the at least one perturbagen.
Embodiment 2016. The method of any one of Embodiments 2012 to 2014, wherein the ratio of the number of monocytes to the number of neutrophils is increased relative to the ratio in the population of progenitor cells prior to contacting with the at least one perturbagen.
Embodiment 2017. The method of Embodiment 2004, wherein the ratio of the number of neutrophils to the number of progenitor cells is increased relative to the ratio obtained from a population of progenitor cells that is not contacted with the at least one perturbagen.
Embodiment 2018. The method of Embodiment 2004, wherein the ratio of the number of neutrophils to the number of progenitor cells is increased relative to the ratio in the population of progenitor cells prior to contacting with the at least one perturbagen.
Embodiment 2019. The method of Embodiment 2004, wherein the ratio of the number of monocytes to the number of progenitor cells is increased relative to the ratio obtained from a population of progenitor cells that is not contacted with the at least one perturbagen.
Embodiment 2020. The method of Embodiment 2004, wherein the ratio of the number of monocytes to the number of progenitor cells is increased relative to the ratio in the population of progenitor cells prior to contacting with the at least one perturbagen.
Embodiment 2021. The method of any one of Embodiments 2004 to 2020, wherein the increase in the number of neutrophils and/or the number of monocytes is due in part to increased cell proliferation of the neutrophils and/or the monocytes.
Embodiment 2022. The method of any one of Embodiments 2004 to 2021, wherein the increase in the number of neutrophils and/or the number of monocytes is due in part to an increased lifespan of the neutrophils and/or the monocytes.
Embodiment 2023. The method of any one of Embodiments 2004 to 2022, wherein the increase in the number of neutrophils and/or the number of monocytes is due in part to reduced cell death among the neutrophils and/or the monocytes.
Embodiment 2024. The method of any one of Embodiments 2004 to 2023, wherein the increase in the number of neutrophils and/or the number of monocytes is due in part to a change of cell state from progenitor cells into the neutrophil and/or monocyte lineage.
Embodiment 2025. The method of any one of Embodiments 2021 to 2024, wherein the number of progenitor cells is decreased.
Embodiment 2026. The method of Embodiment 2025, wherein the decrease in the number of progenitor cells is due in part to decreased cell proliferation of the progenitor cells.
Embodiment 2027. The method of Embodiment 2025 or Embodiment 2026, wherein the decrease in the number of progenitor cells is due in part to a decreased lifespan of the progenitor cells.
Embodiment 2028. The method of any one of Embodiments 2025 to 2027, wherein the decrease in the number of progenitor cells is due in part to increased cell death among the progenitor cells.
Embodiment 2029. The method of any one of Embodiments 2025 to 2028, wherein the decrease in the number of progenitor cells is relative to the number of progenitor cells in a population of progenitor cells that is not contacted with the at least one perturbagen.
Embodiment 2030. The method of any one of Embodiments 2025 to 2029, wherein the decrease in the number of progenitor cells is relative to the number of progenitor cells in the population prior to contacting with the at least one perturbagen.
Embodiment 2031. The method of any one of Embodiments 2025 to 2030, wherein the decrease in the number of progenitor cells is due to a change of cell state from a progenitor cell into the neutrophil lineage and/or monocyte lineage.
Embodiment 2032. The method of any one of Embodiments 2021 to 2024, wherein the number of progenitor cells is increased.
Embodiment 2033. The method of Embodiment 2032, wherein the increase in the number of progenitor cells is due in part to increased cell proliferation of the progenitor cells.
Embodiment 2034. The method of Embodiment 2032 or Embodiment 2033, wherein the increase in the number of progenitor cells is due in part to an increased lifespan of the progenitor cells.
Embodiment 2035. The method of any one of Embodiments 2032 to 2034, wherein the increase in the number of progenitor cells is due in part to decreased cell death among the progenitor cells.
Embodiment 2036. The method of any one of Embodiments 2032 to 2035, wherein the increase in the number of progenitor cells is relative to the number of progenitor cells in a population of progenitor cells that is not contacted with the at least one perturbagen.
Embodiment 2037. The method of any one of Embodiments 2032 to 2035, wherein the increase in the number of progenitor cells is relative to the number of progenitor cells in the population prior to contacting with the at least one perturbagen.
Embodiment 2038. The method of any one of Embodiments 2021 to 2024, wherein the number of CD66b+neutrophil progenitors, CD64+monocytic progenitors, neutrophilic promyelocytes, neutrophilic myelocytes, neutrophilic metamyelocytes, and/or neutrophilic band cells is increased and/or the number of CD34+CD38+/− cells are increased about two days after contacting the population of cells comprising a CD34+ cell with the at least one perturbagen and/or the number of CD34-CD38+/− cells are increased about five days after contacting the population of cells comprising a CD34+ cell with the at least one perturbagen.
Embodiment 2039. The method of Embodiment 2038, wherein the ratio of the number of CD66b+ neutrophil progenitors, CD64+monocytic progenitors, neutrophilic promyelocytes, neutrophilic myelocytes, neutrophilic metamyelocytes, neutrophilic band cells, CD34+CD38+/− cells, and/or CD34-CD38+/− cells to the number of progenitor cells is increased relative to the ratio obtained from a population of progenitor cells that is not contacted with the at least one perturbagen.
Embodiment 2040. The method of Embodiment 2038, wherein the ratio of the number of CD66b+ neutrophil progenitors, CD64+monocytic progenitors, neutrophilic promyelocytes, neutrophilic myelocytes, neutrophilic metamyelocytes, neutrophilic band cells, CD34+CD38+/− cells, and/or CD34-CD38+/− cells to the number of progenitor cells is increased relative to the ratio in the population of progenitor cells prior to contacting with the at least one perturbagen.
Embodiment 2041. The method of any one of Embodiments 2021 to 2024, wherein the number of monoblasts and/or promonocytes is increased.
Embodiment 2042. The method of Embodiment 2041, wherein the ratio of the number of monoblasts and/or promonocytes to the number of progenitor cells is increased relative to the ratio obtained from a population of progenitor cells that is not contacted with the at least one perturbagen.
Embodiment 2043. The method of Embodiment 2041, wherein the ratio of the number of monoblasts and/or promonocytes to the number of progenitor cells is increased relative to the ratio in the population of progenitor cells prior to contacting with the at least one perturbagen.
Embodiment 2044. The method of any one of Embodiments 2021 to 2024, wherein the number of granulocyte-monocyte progenitor (GMP) cells is increased.
Embodiment 2045. The method of Embodiment 2044, wherein the ratio of the number of GMP cells to the number of progenitor cells for the GMP cell is increased relative to the ratio obtained from a population of progenitor cells that is not contacted with the at least one perturbagen.
Embodiment 2046. The method of Embodiment 2044, wherein the ratio of the number of GMP cells to the number of progenitor cells for the GMP cell is increased relative to the ratio in the population of progenitor cells prior to contacting with the at least one perturbagen.
Embodiment 2047. The method of Embodiment 2046, wherein the progenitor cell for the GMP cell is a common myeloid progenitor (CMP).
Embodiment 2048. The method of Embodiment 2044, wherein the number of monocyte-dendritic cell progenitor (MDP) cells is decreased.
Embodiment 2049. The method of Embodiment 2048, wherein the ratio of the number of GMP cells to the number of MDP cells is increased relative to the ratio obtained from a population of progenitor cells that is not contacted with the at least one perturbagen.
Embodiment 2050. The method of Embodiment 2048, wherein the ratio of the number of GMP cells to the number of MDP cells is increased relative to the ratio in the population of progenitor cells prior to contacting with the at least one perturbagen.
Embodiment 2051. The method of any one of Embodiments 2001 to 2050, wherein the at least one perturbagen selected from Table 3, or a variant thereof, comprises at least 2, at least 3, at least 4, at least 5, at least 6, at least 7, at least 8, at least 9, or 10 perturbagens selected from Table 3, or variants thereof.
Embodiment 2052. The method of Embodiment 2051, wherein the at least one perturbagen promotes the monocyte lineage.
Embodiment 2053. The method of Embodiment 2051, wherein the at least one perturbagen promotes the neutrophil lineage.
Embodiment 2054. The method of any one of Embodiments 2001 to 2053, wherein contacting the population of progenitor cells occurs in vitro or ex vivo.
Embodiment 2055. The method of any one of Embodiments 2001 to 2053, wherein contacting the population of progenitor cells occurs in vivo in a subject.
Embodiment 2056. The method of Embodiment 2055, wherein the subject is a human.
Embodiment 2057. The method of Embodiment 2056, wherein the human is an adult human.
Embodiment 2058. A perturbagen for use in the method of any one of Embodiments 2001 to 2057.
Embodiment 2059. A pharmaceutical composition comprising the perturbagen of Embodiment 2058.
Embodiment 2060. A method for promoting the formation of a neutrophil, a monocyte, or an immediate progenitor thereof, comprising: exposing a starting population of stem/progenitor cells comprising a non-lineage committed CD34+ cell to a perturbagen; wherein the exposing promotes the transition of the starting population of stem/progenitor cells into a granulocyte monocyte progenitor, neutrophil progenitor, monocyte progenitor or other lineage associated progenitor thereof.
Embodiment 2061. A method of increasing a quantity of neutrophils, monocytes, or immediate progenitors thereof, comprising: exposing a starting population of stem/progenitor cells comprising a non-lineage committed CD34+ cell to a pharmaceutical composition that promotes the formation of lineage specific progenitor population selected from granulocyte monocyte progenitor, neutrophil progenitor, monocyte progenitor, or other lineage-associated progenitors, the pharmaceutical composition promoting the transition of a primitive stem/progenitor population into the lineage specific progenitor population that has the capacity to differentiate into neutrophils, monocytes or immediate progenitors thereof, wherein the pharmaceutical composition comprises at least one perturbagen selected from Table 3, or a variant thereof.
Embodiment 2062. A method for treating a disease or disorder characterized by an abnormal ratio of neutrophils to monocytes, comprising: (a) administering to a patient in need thereof a therapeutically effective amount of at least one perturbagen selected from Table 3, or a variant thereof, or (b) administering to a patient in need thereof a cell, the cell having been contacted with at least one perturbagen selected from Table 3, or a variant thereof.
Embodiment 2063. The method of Embodiment 2062, wherein the abnormal ratio comprises an increased number of neutrophils and/or a decreased number of monocytes.
Embodiment 2064. The method of Embodiment 2062, wherein the abnormal ratio comprises a decreased number of neutrophils and/or an increased number of monocytes.
Embodiment 2065. The method of Embodiment 2062, wherein the abnormal ratio comprises a decreased number of neutrophils and a decreased number of monocytes.
Embodiment 2066. The method of any one of Embodiments 2062 to 2065, wherein the administering is directed to the bone marrow of the patient.
Embodiment 2067. The method of Embodiment 2066, wherein the administering is via intraosseous injection or intraosseous infusion.
Embodiment 2068. The method of any one of Embodiments 2062 to 2067, wherein the administering the cell is via intravenous injection or intravenous infusion.
Embodiment 2069. The method of any one of Embodiments 2062 to 2068, wherein the administering is simultaneously or sequentially to one or more mobilization agents.
Embodiment 2070. The method of any one of Embodiments 2062 to 2069, wherein the disease or disorder characterized by an abnormal ratio of neutrophils to monocytes is selected from leukopenia, leukocytosis, agranulocytosis, granulocytosis, monocytopenia, monocytosis, neutropenia, and neutrophilia, leukemia (e.g., Chronic myeloid leukemia (CML), Chronic myelomonocytic leukemia (CMML), and Acute myeloblastic leukemia (AML), including AML-M5), cancer induced neutropenia, drug induced neutropenia, infection induce neutropenia, lupus (and other autoimmune disorders showing neutropenia), myelofibrosis, Chronic idiopathic neutropenia (CIN), and/or

Autoimmune idiopathic neutropenia (AlN).
Embodiment 2071. The method of any one of Embodiments 2062 to 2070, wherein the disease or disorder characterized by an abnormal ratio of neutrophils to monocytes is related to an autoimmune disease, a bone-marrow transplant, a cancer, a genetic disease, a bacterial infection, a viral infection, inflammatory bowel disease, tissue necrosis (e.g., myocardial infarction and burns), a previous or concurrent anti-cancer treatment, and/or, a previous or concurrent immune suppressive treatment.
Embodiment 2072. A method for treating a disease or disorder characterized by an abnormal ratio of neutrophils and/or monocytes to progenitor cells, comprising: (a) administering to a patient in need thereof at least one perturbagen selected from Table 3, or a variant thereof, or (b) administering to a patient in need thereof a cell, the cell having been contacted with at least one perturbagen selected from Table 3, or a variant thereof.
Embodiment 2073. The method of Embodiment 2072, wherein the abnormal ratio comprises a decreased number of neutrophils and/or a decreased number of monocytes and/or an increased number of progenitor cells.
Embodiment 2074. The method of Embodiment 2073, wherein the abnormal ratio comprises a decreased number of neutrophils and an increased number of progenitor cells.
Embodiment 2075. The method of Embodiment 2073, wherein the abnormal ratio comprises a decreased number of monocytes and an increased number of progenitor cells.
Embodiment 2076. The method of Embodiment 2073, wherein the abnormal ratio comprises a decreased number of neutrophils and a decreased number of monocytes.
Embodiment 2077. The method of Embodiment 2076, wherein the abnormal ratio comprises an increased number of progenitor cells.
Embodiment 2078. The method of any one of Embodiments 2072 to 2077, wherein the administering is directed to the bone marrow of the patient.
Embodiment 2079. The method of Embodiment 2078, wherein the administering is via intraosseous injection or intraosseous infusion.
Embodiment 2080. The method of any one of Embodiments 2072 to 2079, wherein the administering the cell is via intravenous injection or intravenous infusion.
Embodiment 2081. The method of any one of Embodiments 2072 to 2080, wherein the administering is simultaneously or sequentially to one or more mobilization agents.
Embodiment 2082. The method of any one of Embodiments 2072 to 2081, wherein the disease or disorder characterized by an abnormal ratio of neutrophils and/or monocytes to progenitor cells is selected from leukopenia, leukocytosis, agranulocytosis, granulocytosis, monocytopenia, monocytosis, neutropenia, and neutrophilia, leukemia (e.g., Chronic myeloid leukemia (CML), Chronic myelomonocytic leukemia (CMML), and Acute myeloblastic leukemia (AML), including AML-M5), cancer induced neutropenia, drug induced neutropenia, infection induce neutropenia, lupus (and other autoimmune disorders showing neutropenia), myelofibrosis, Chronic idiopathic neutropenia (CIN), and/or Autoimmune idiopathic neutropenia (AlN).
Embodiment 2083. The method of any one of Embodiments 2072 to 2082, wherein the disease or disorder characterized by an abnormal ratio of neutrophils and/or monocytes to progenitor cells is related to an autoimmune disease, a bone-marrow transplant, a cancer, a genetic disease, a bacterial infection, a viral infection, inflammatory bowel disease, tissue necrosis (e.g., myocardial infarction and burns), a previous or concurrent anti-cancer treatment, and/or, a previous or concurrent immune suppressive treatment.
Embodiment 2084. A method for treating monocytopenia, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 2085. A method for treating monocytosis, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 2086. A method for treating neutropenia, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 2087. A method for treating neutrophilia, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 2088. A method for treating agranulocytosis, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 2089. The method of Embodiment 2088, wherein the agranulocytosis is monocytopenia.
Embodiment 2090. The method of Embodiment 2088, wherein the agranulocytosis is neutropenia.
Embodiment 2091. A method for treating granulocytosis, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 2092. The method of Embodiment 2091, wherein the agranulocytosis is monocytosis.
Embodiment 2093. The method of Embodiment 2091, wherein the agranulocytosis is neutrophilia.
Embodiment 2094. A method of treating a disorder selected from drug-induced neutropenia, a side-effect of bone marrow transplantation, or MonoMAC syndrome, comprising: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.
Embodiment 2095. The method of Embodiment 2094, wherein the drug-induced neutropenia in associated with a prior or concurrent chemotherapy and/or radiotherapy treatment.
Embodiment 2096. The method of any one of Embodiments 2084 to 2095, wherein the therapeutically effective amount of the at least one perturbagen provides an increase in the number of neutrophils and/or the number of monocytes in a sample from the human subject relative to the number of neutrophils and/or the number of monocytes in a sample from the human subject prior to administering the at least one perturbagen.
Embodiment 2097. The method of any one of Embodiments 2084 to 2096, wherein the therapeutically effective amount of the at least one perturbagen provides an increase in the ratio of the number of neutrophils to the number of monocytes in a sample from the human subject relative to the ratio in a sample from the human subject prior to administering the at least one perturbagen.
Embodiment 2098. The method of any one of Embodiments 2084 to 2097, wherein the therapeutically effective amount of the at least one perturbagen provides an increase in the ratio of the number of monocytes to the number of neutrophils in a sample from the human subject relative to the ratio in a sample from the human subject prior to administering the at least one perturbagen.
Embodiment 2099. The method of any one of Embodiments 2084 to 2098, wherein the therapeutically effective amount of the at least one perturbagen provides an increase in the ratio of the number of neutrophils and/or the number of monocytes to the number of progenitor cells in a sample from the human subject relative to the ratio in a sample from the human subject prior to administering the at least one perturbagen.
Embodiment 2100. The method of any one of Embodiments 2084 to 2099, wherein the administering the therapeutically effective amount of the at least one perturbagen is directed to the bone marrow of the patient.
Embodiment 2101. The method of Embodiment 2100, wherein the administering is via intraosseous injection or intraosseous infusion.
Embodiment 2102. The method of any one of Embodiments 2084 to 2101, wherein the administering the cell is via intravenous injection or intravenous infusion.
Embodiment 2103. The method of any one of Embodiments 2084 to 2101, wherein the administering is simultaneously or sequentially to one or more mobilization agents.
Embodiment 2104. The method of any one of Embodiments 2084 to 2103, wherein the administering occurs about once per day for one or more days.
Embodiment 2105. The method of any one of Embodiments 2084 to 2104, wherein the administering occurs more than once per day for one or more days.
Embodiment 2106. The method of any one of Embodiments 2084 to 2105, wherein the administering occurs at most once per day for one or more days.
Embodiment 2107. The method of any one of Embodiments 2084 to 2106, wherein the administering occurs substantially continuously per administration period.
Embodiment 2108. The method of any one of Embodiments 2084 to 2107, wherein the patient was selected by steps comprising: obtaining from the patient having the disease or disorder a sample of cells comprising a non-lineage committed CD34+ cell; and contacting the sample of cells with least one perturbagen selected from Table 3, or a variant thereof, wherein the at least one perturbagen increases the number of monocytes and/or neutrophils or a progenitor thereof in a culture from the sample of cells.
Embodiment 2109. A method for selecting the patient of any one of Embodiments 2084 to 2108, comprising: obtaining from a subject having the disease or disorder a sample of cells comprising a non-lineage committed CD34+ cell; and contacting the sample of cells with least one perturbagen selected from Table 3, or a variant thereof, wherein when the at least one perturbagen increases the number of monocytes and/or neutrophils or a progenitor thereof in a culture from the sample of cells, the subject is selected as a patient.
Embodiment 2110. Use of the perturbagen of Table 3, or a variant thereof in the manufacture of a medicament for treating a disease or disorder characterized by an abnormal ratio of neutrophils to monocytes.
Embodiment 2111. Use of the perturbagen of Table 3, or a variant thereof in the manufacture of a medicament for treating a disease or disorder characterized by an abnormal ratio of neutrophils and/or monocytes to progenitor cells.
Embodiment 2112. A method of identifying a perturbagen for promoting the transition of a starting population of progenitor cells into neutrophils, monocytes or immediate progenitors thereof, the method comprising: exposing the starting population of progenitor cells to a candidate perturbagen; determining a change in cell state of the cells in the population of progenitor cells into neutrophils, monocytes or immediate progenitors thereof following exposure of the population of cells to the perturbagen, wherein the candidate perturbagen capable changing the cell state of the cells in the population progenitor cells is identified as a perturbagen for promoting the transition of a starting population of progenitor cells into neutrophils, monocytes or immediate progenitors thereof.
Embodiment 2113. A method for making a therapeutic agent for a disease or disorder selected from leukopenia, leukocytosis, agranulocytosis, granulocytosis, monocytopenia, monocytosis, neutropenia, and neutrophilia, leukemia, cancer induced neutropenia, drug induced neutropenia, infection induce neutropenia, lupus (and other autoimmune disorders showing neutropenia), myelofibrosis, Chronic idiopathic neutropenia (CIN), and Autoimmune idiopathic neutropenia (AlN), comprising: (a) identifying a perturbagen capable of acting as a therapeutic agent for therapy according to the method of Embodiment 2112 and (b) formulating the therapeutic agent for the treatment of the disease or disorder.

### Methods for Inhibiting a Change in Cell State

### Genes Signatures

Cell state transitions (i.e., a transition in a cell's state from a first cell state to a second cell state, e.g., differentiation) are characterized by a change in expression of genes in the cell. Changes in gene expression may be quantified as the increase in mRNA expressed for a specific gene or a decrease in mRNA expressed for another specific gene; especially significant here may be mRNAs that encode transcription factors. Collectively, the sum of multiple differences in gene expression in one cell type or cells of one lineage relative to another cell type or cells of another lineage are referred to herein as a gene signature.

Any one of a number of methods and metrics may be used to identify gene signatures. Non-limiting examples include single cell and bulk RNA sequencing with or without prior cell sorting (e.g., fluorescence activated cell sorting (FACS) and flow cytometry). When developing a gene signature, it may useful to first characterize the cell type or cells of a specific lineage by surface proteins that are characteristic of the cell type or cells of a specific lineage. Illustrative surface proteins are listed in Table 9 and Table 11.

Knowing the gene signature for each cell type or cells of a specific lineage provides insight into what genes impact or are associated with the process of transition to other cell types and/or differentiation of progenitor cells.

Gene signatures can be used to identify particular cells as being on-lineage, and other cells as being “progenitor” cells or intermediate cells along a transition trajectory towards the on-lineage cell type.

FIG. 1A, shows annotated clusters that associate gene signature with cell types or cells of a specific lineage. Differential gene signatures for the 10 to 9 transition, i.e., from a non-lineage committed CD34+ progenitor cell to cells of the granulocyte monocyte progenitor (GMP) lineage, were used to predict perturbations relevant to the transition. Those genes that are differentially expressed and positively associated with inhibiting granulocyte monocyte progenitor (GMP) lineage progression and/or GMP differentiation and negatively associated with the promoting of GMP lineage progression and/or GMP differentiation are listed in Table 4:

TABLE 4

| | Gene Name | GeneID |
|---|---|---|
| 1 | HMGA1 | 3159 |
| 2 | SSBP4 | 170463 |
| 3 | LSM4 | 25804 |
| 4 | CDK4 | 1019 |
| 5 | SMARCB1 | 6598 |
| 6 | AES | 166 |
| 7 | GTF3A | 2971 |
| 8 | RAB8A | 4218 |
| 9 | RNPS1 | 10921 |
| 10 | THRAP3 | 9967 |
| 11 | SNRPB | 6628 |
| 12 | CARHSP1 | 23589 |
| 13 | DEK | 7913 |
| 14 | DNMT1 | 1786 |
| 15 | POLR2J | 5439 |
| 16 | DEAF1 | 10522 |
| 17 | PHF5A | 84844 |
| 18 | E2F4 | 1874 |
| 19 | IFI16 | 3428 |
| 20 | SSRP1 | 6749 |
| 21 | PSMC5 | 5705 |
| 22 | HHEX | 3087 |
| 23 | BTF3 | 689 |
| 24 | LMO2 | 4005 |
| 25 | HMG20B | 10362 |
| 26 | HOXA9 | 3205 |
| 27 | POLR2C | 5432 |
| 28 | POLR2K | 5440 |
| 29 | LYL1 | 4066 |
| 30 | MYBL2 | 4605 |
| 31 | SAP18 | 10284 |
| 32 | TFDP2 | 7029 |
| 33 | SMARCA4 | 6597 |
| 34 | POLR21 | 5438 |
| 35 | PFDN1 | 5201 |
| 36 | MLLT3 | 4300 |
| 37 | TAF10 | 6881 |
| 38 | DRAP1 | 10589 |
| 39 | GATA2 | 2624 |
| 40 | ABT1 | 29777 |
| 41 | TAL1 | 6886 |
| 42 | HDAC3 | 8841 |
| 43 | GFI1B | 8328 |
| 44 | TFDP1 | 7027 |
| 45 | NACA | 4666 |
| 46 | NCOR1 | 9611 |
| 47 | PDLIM1 | 9124 |
| 48 | PDCD7 | 10081 |
| 49 | HSF1 | 3297 |
| 50 | ZDHHC16 | 84287 |
| 51 | TFAM | 7019 |
| 52 | TIAL 1 | 7073 |
| 53 | ETS2 | 2114 |
| 54 | MEF2C | 4208 |
| 55 | BAZ1B | 9031 |
| 56 | NR1H2 | 7376 |
| 57 | PSMC3 | 5702 |
| 58 | HOXA7 | 3204 |
| 59 | MYC | 4609 |
| 60 | UBTF | 7343 |
| 61 | PTGES2 | 80142 |
| 62 | KEAP1 | 9817 |
| 63 | NMI | 9111 |
| 64 | FHL2 | 2274 |
| 65 | E2F1 | 1869 |
| 66 | TRIP6 | 7205 |
| 67 | TAF6 | 6878 |
| 68 | SOX4 | 6659 |
| 69 | STRAP | 11171 |
| 70 | SSBP2 | 23635 |
| 71 | ARID5B | 84159 |
| 72 | HSBP1 | 3281 |

At the time of filing the present disclosure, the World Wide Web at ncbi.nlm.nih.gov/gene provides a description of and the nucleic acid sequence for each GeneID listed in Table 4; the contents of each of which is incorporated herein by reference in its entirety.

In contrast, those genes that are differentially expressed and positively associated with promoting the inhibiting GMP lineage progression and/or differentiation are listed in Table 5:

TABLE 5

| | Gene Name | GeneID |
|---|---|---|
| 1 | MYB | 4602 |
| 2 | NCOA4 | 8031 |
| 3 | CEBPD | 1052 |
| 4 | CEBPA | 1050 |
| 5 | TLE4 | 7091 |
| 6 | DACH1 | 1602 |
| 7 | SMARCA2 | 6595 |
| 8 | GFI1 | 2672 |
| 9 | MAZ | 4150 |
| 10 | TLE1 | 7088 |
| 11 | ELL2 | 22936 |
| 12 | ARID1A | 8289 |
| 13 | CITED4 | 163732 |
| 14 | ARID3A | 1820 |
| 15 | PTMA | 5757 |
| 16 | STAT2 | 6773 |
| 17 | NFIL3 | 4783 |
| 18 | SERTAD2 | 9792 |
| 19 | RCOR1 | 23186 |
| 20 | BTBD11 | 121551 |
| 21 | TRERF1 | 55809 |
| 22 | NOTCH2 | 4853 |
| 23 | CCNT1 | 904 |
| 24 | UHRF2 | 115426 |
| 25 | ASB1 | 51665 |
| 26 | BASP1 | 10409 |
| 27 | FOXK1 | 221937 |
| 28 | MAPK1 | 5594 |
| 29 | CREB1 | 1385 |
| 30 | IRX3 | 79191 |
| 31 | STAT4 | 6775 |
| 32 | SCX | 642658 |
| 33 | HIPK2 | 28996 |
| 34 | EZH1 | 2145 |
| 35 | ANK1 | 286 |
| 36 | ENPP2 | 5168 |
| 37 | KLF4 | 9314 |
| 38 | BCL6 | 604 |
| 39 | UTF1 | 8433 |
| 40 | NFKB1 | 4790 |
| 41 | PPP3CB | 5532 |
| 42 | RUNX1 | 861 |
| 43 | NCOA3 | 8202 |
| 44 | CITED2 | 10370 |
| 45 | RNF38 | 152006 |
| 46 | MAGED1 | 9500 |
| 47 | TNFAIP3 | 7128 |
| 48 | ZFPM1 | 161882 |
| 49 | HIVEP1 | 3096 |

At the time of filing the present disclosure, the World Wide Web at ncbi.nlm.nih.gov/gene provides a description of and the nucleic acid sequence for each GeneID listed in Table 5; the contents of each of which is incorporated herein by reference in its entirety.

### Perturbagens

A perturbagen useful in the present disclosure can be a small molecule, a biologic, a protein, a nucleic acid, such as a cDNA over-expressing a wild-type gene or an mRNA encoding a wild-type gene, or any combination of any of the foregoing. Illustrative perturbagens useful in the present disclosure and capable of preventing progression of a progenitor cell into and/or reducing the likelihood that a progenitor cell will progress into the granulocyte monocyte progenitor (GMP) lineage are listed in Table 6.

TABLE 6

| | | Molecular | Effective in | |
|---|---|---|---|---|
| Perturbagen | Molecular | weight | vitro | |
| No. | formula | (g/mol) | concentration | |
| 1 | C₃₆H₅₆O₈ | 616.8 | 10 | μM |
| 2 | C₁₆H₁₁BrN₂O | 327.17 | 20 | μM |
| 3 | C₁₇H₂₄N₄O₂S₂ | 380.5 | 1 | μM |
| 4 | C₁₆H₂₄O₄ | 280.36 | 10 | μM |
| 5 | C₃₀H₂₈FN₇O₃ | 553.6 | 10.0 | μM |
| 6 | C₂₂H₂₁F₃N₆O₃S | 506.5 | 10.0 | μM |
| 7 | C₂₂H₂₈N₄O₆ | 444.5 | 10 | μM |
| 8 | C₂₈H₂₆N₄O₃ | 466.5 | 1.0 | μM |
| 9 | C₂₀H₂₄O₆ | 360.4 | 1.11 | μM |
| 10 | C₁₈H₁₈ClN₅O | 355.8 | 10 | μM |

In various embodiments herein, a perturbagen encompasses the perturbagens named in Table 6. Thus, the named perturbagens of Table 6 represent examples of perturbagens of the present disclosure.

In Table 6, the effective in vitro concentration is the concentration of a perturbagen that is capable of increasing gene expression in a progenitor cell and/or inhibiting the natural change in cell state of a progenitor cell towards a cell of the GMP lineage, as assayed, at least, by single cell gene expression profiling (GEP). Although the concentrations were determined in an in vitro assay, the concentrations may be relevant to a determination of in vivo dosages.

In embodiments, a perturbagen used in the present disclosure is a variant of a perturbagen of Table 6. A variant may be a derivative, analog, enantiomer or a mixture of enantiomers thereof or a pharmaceutically acceptable salt, solvate, hydrate, co-crystal, clathrate, or polymorph of the perturbagen of Table 6. A variant of a perturbagen of Table 6 retains the biological activity of the perturbagen of Table 6.

### Methods and Perturbagens for Directing a Change in Cell State

Particular cellular changes in cell state can be matched to differential gene expression (which collectively define a gene signature), caused by exposure of a cell to a perturbagen; conversely, inhibiting a change in cell state of can be matched with a different gene signature. A change in cell state may be from one progenitor cell type to another progenitor cell type. For example, a common myeloid progenitor (CMP) may change to a granulocyte monocyte progenitor (GMP). A change in cell state may be from an upstream progenitor cell to a downstream progenitor cell. Lastly, inhibiting a change in cell state may be the prevention of the natural progression from one state to another. As examples, a change in cell state may be a block in the transition from an upstream progenitor cell to a downstream progenitor cell and/or inhibiting the change in cell state may be a block to the differentiation of a final non-differentiated cell into a differentiated cell, e.g., a neutrophil and a monocyte, which would have naturally occurred absent the perturbagen. Thus, as used herein, inhibiting a change in cell state is where a cell is prevented from acquiring the state that it would have obtained under natural conditions.

An aspect of the present disclosure is a method for inhibiting a change in cell state of a non-lineage committed CD34+ progenitor cell. The method comprising a step of contacting a population of cells comprising a non-lineage committed CD34+ progenitor cell with at least one perturbagen selected from Table 6, or a variant thereof. In this aspect, the at least one perturbagen is capable of altering a gene signature in the progenitor cell.

As also disclosed herein, particular natural cellular changes in cell state are inhibited by exposure of a cell to a perturbagen. A natural change in cell state may be from one progenitor cell type into another progenitor cell type. For example, a common myeloid progenitor (CMP) may naturally change to a granulocyte monocyte progenitor (GMP). A natural change in cell state may be from an upstream progenitor cell to a downstream progenitor cell. Lastly, a change in cell state may be from the final non-differentiated cell into a differentiated cell, e.g., a neutrophil and a monocyte.

An aspect of the present disclosure is a method for inhibiting a change in cell state of a non-lineage committed CD34+ progenitor cell. The method comprising a step of contacting a population of cells comprising a non-lineage committed CD34+ progenitor cell with at least one perturbagen selected from Table 6, or a variant thereof. In this aspect, the at least one perturbagen is capable of inhibiting a change in cell state of a progenitor cell, which is a non-lineage committed CD34+ cell.

Another aspect of the present disclosure is a method for inhibiting a change in cell state of a non-lineage committed CD34+ progenitor cell. The method comprising a step of contacting a population of cells comprising a non-lineage committed CD34+ progenitor cell with at least one perturbagen capable of altering a gene signature in the progenitor cell. In this aspect, altering the gene signature comprises an increase in expression and/or activity in the progenitor cell of one or more genes selected from Table 4 and/or a decrease in expression and/or activity in the progenitor cell of one or more genes selected from Table 5.

Yet another aspect of the present disclosure is a method for inhibiting a change in cell state of a non-lineage committed CD34+ progenitor cell. The method comprising a step of contacting a population of cells comprising a non-lineage committed CD34+ progenitor cell with at least one perturbagen selected from Table 6, or a variant thereof, and capable of altering a gene signature in the progenitor cell. In this aspect, altering the gene signature comprises an increase in expression and/or activity in the progenitor cell of one or more genes selected from Table 4 and/or a decrease in expression and/or activity in the progenitor cell of one or more genes selected from Table 5.

In embodiments, the non-lineage committed CD34+ cell is a hematopoietic stem and progenitor cell (HSPC).

In embodiments, inhibiting the change in cell state provides a decrease in the number of neutrophils and/or the number of monocytes.

In embodiments, the decrease in the number of neutrophils and/or the number of monocytes is relative to the number of neutrophils and/or the number of monocytes obtained from a population of progenitor cells that is not contacted with the at least one perturbagen.

In embodiments, the decrease in the number of neutrophils and/or the number of monocytes is relative to the population of progenitor cells prior to contacting with the at least one perturbagen.

In embodiments, inhibiting the change in cell state provides a decrease in the number of neutrophils. In embodiments, inhibiting the change in cell state provides a decrease in the number of monocytes. In embodiments, inhibiting the change in cell state does not provide a substantial increase in the number of monocytes and/or provides a decrease in the number of monocytes. In embodiments, the ratio of the number of neutrophils to the number of monocytes is decreased relative to the ratio obtained from a population of progenitor cells that is not contacted with the at least one perturbagen. In embodiments, the ratio of the number of neutrophils to the number of monocytes is decreased relative to the ratio in the population of progenitor cells prior to contacting with the at least one perturbagen.

In embodiments, inhibiting the change in cell state provides a decrease in the number of monocytes. In embodiments, inhibiting the change in cell state provides a decrease in the number of neutrophils. In embodiments, inhibiting the change in cell state does not provide a substantial increase in the number of neutrophils and/or provides a decrease in the number of neutrophils. In embodiments, the ratio of the number of monocytes to the number of neutrophils is decreased relative to the ratio obtained from a population of progenitor cells that is not contacted with the at least one perturbagen. In embodiments, the ratio of the number of monocytes to the number of neutrophils is decreased relative to the ratio in the population of progenitor cells prior to contacting with the at least one perturbagen.

In embodiments, the ratio of the number of neutrophils to the number of progenitor cells is decreased relative to the ratio obtained from a population of progenitor cells that is not contacted with the at least one perturbagen.

In embodiments, the ratio of the number of neutrophils to the number of progenitor cells is decreased relative to the ratio in the population of progenitor cells prior to contacting with the at least one perturbagen.

In embodiments, the ratio of the number of monocytes to the number of progenitor cells is decreased relative to the ratio obtained from a population of progenitor cells that is not contacted with the at least one perturbagen.

In embodiments, the ratio of the number of monocytes to the number of progenitor cells is decreased relative to the ratio in the population of progenitor cells prior to contacting with the at least one perturbagen.

In embodiments, the decrease in the number of neutrophils and/or the number of monocytes is due in part to decreased cell proliferation of the neutrophils and/or the monocytes.

In embodiments, the decrease in the number of neutrophils and/or the number of monocytes is due in part to a decreased lifespan of the neutrophils and/or the monocytes. In embodiments, the decrease in the number of neutrophils and/or the number of monocytes is due in part to increased cell death among the neutrophils and/or the monocytes. Methods for determining a decreased lifespan of a specific cell type or an increase in cell death is well known in the art. As examples, markers for dying cells, e.g., caspases can be detected, or dyes for dead cells, e.g., methylene blue, may be used.

In embodiments, the decrease in the number of neutrophils and/or the number of monocytes is due in part to inhibiting a change of cell state from progenitor cells into the neutrophil and/or monocyte lineage.

In embodiments, the number of progenitor cells is increased. In embodiments, the increase in the number of progenitor cells is due in part to increased cell proliferation of the progenitor cells. In embodiments, the increase in the number of progenitor cells is due in part to an increased lifespan of the progenitor cells. In embodiments, the increase in the number of progenitor cells is due in part to decreased cell death among the progenitor cells. In embodiments, the increase in the number of progenitor cells is relative to the number of progenitor cells in a population of progenitor cells that is not contacted with the at least one perturbagen. In embodiments, the increase in the number of progenitor cells is relative to the number of progenitor cells in the population prior to contacting with the at least one perturbagen. In embodiments, the increase in the number of progenitor cells is due to a change and/or inhibiting a change of cell state from a progenitor cell into the neutrophil lineage and/or monocyte lineage.

Methods for counting cells are well known in the art. Non-limiting examples include hemocytometry, flow cytometry, and cell sorting techniques, e.g., fluorescence activated cell sorting (FACS).

In embodiments, the number of CD66b+ neutrophil progenitors, CD64+monocytic progenitors, neutrophilic promyelocytes, neutrophilic myelocytes, neutrophilic metamyelocytes, and/or neutrophilic band cells is decreased and/or the number of CD34+CD38+/− cells is decreased about two days after contacting the population of cells comprising a CD34+ cell with the at least one perturbagen, and/or the number of CD34-CD38+/− cells are decreased about five days after contacting the population of cells comprising a CD34+ cell with the at least one perturbagen. In embodiments, the ratio of the number of CD66b+ neutrophil progenitors, CD64+monocytic progenitors, neutrophilic promyelocytes, neutrophilic myelocytes, neutrophilic metamyelocytes, neutrophilic band cells, CD34+CD38+/− cells, and/or CD34-CD38+/− cells to the number of progenitor cells is decreased relative to the ratio obtained from a population of progenitor cells that is not contacted with the at least one perturbagen. In embodiments, the ratio of the number of CD66b+ neutrophil progenitors, CD64+monocytic progenitors, neutrophilic promyelocytes, neutrophilic myelocytes, neutrophilic metamyelocytes, neutrophilic band cells, CD34+CD38+/− cells, and/or CD34-CD38+/− cells to the number of progenitor cells is decreased relative to the ratio in the population of progenitor cells prior to contacting with the at least one perturbagen.

In embodiments, the number of monoblasts and/or promonocytes is decreased. In embodiments, the ratio of the number of monoblasts and/or promonocytes to the number of progenitor cells is decreased relative to the ratio obtained from a population of progenitor cells that is not contacted with the at least one perturbagen. In embodiments, the ratio of the number of monoblasts and/or promonocytes to the number of progenitor cells is decreased relative to the ratio in the population of progenitor cells prior to contacting with the at least one perturbagen.

In embodiments, the number of GMP cells is decreased. In embodiments, the ratio of the number of GMP cells to the number of progenitor cells for the GMP cell is decreased relative to the ratio obtained from a population of progenitor cells that is not contacted with the at least one perturbagen. In embodiments, the ratio of the number of GMP cells to the number of progenitor cells for the GMP cell is decreased relative to the ratio in the population of progenitor cells prior to contacting with the at least one perturbagen. In embodiments, the progenitor cell for the GMP cell is a common myeloid progenitor (CMP). In embodiments, the number of monocyte-dendritic cell progenitor (MDP) cells is increased. In embodiments, the ratio of the number of GMP cells to the number of MDP cells is decreased relative to the ratio obtained from a population of progenitor cells that is not contacted with the at least one perturbagen.

In embodiments, the ratio of the number of GMP cells to the number of MDP cells is decreased relative to the ratio in the population of progenitor cells prior to contacting with the at least one perturbagen.

In embodiments, the at least one perturbagen selected from Table 6, or a variant thereof, comprises at least 2, at least 3, at least 4, at least 5, at least 6, at least 7, at least 8, at least 9, or 10 perturbagens selected from Table 6, or variants thereof.

In any of the aspects or embodiments disclosed herein, the at least one perturbagen is selected Table 6.

In embodiments, the at least one perturbagen comprises one or more perturbagens selected from Table 6, or variants thereof. In embodiments, the at least one perturbagen prevents progression of a progenitor cell into and/or reduces the likelihood that a progenitor cell will progress into the monocyte lineage. In embodiments, the at least one perturbagen prevents progression of a progenitor cell into and/or reduces the likelihood that a progenitor cell will progress into the neutrophil lineage.

In embodiments, altering the gene signature comprises increased expression and/or increased activity in the progenitor cell of one or more genes selected from Table 4. In embodiments, the one or more genes selected from Table 4 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, or 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, or 72 genes selected from Table 4. In embodiments, the one or more genes selected from Table 4 comprises at least one of HMGA1, SSBP4, LSM4, CDK4, SMARCB1, AES, GTF3A, RAB8A, RNPS1, THRAP3, and SNRPB. In embodiments, the one or more genes selected from Table 4 comprises at least one of HMGA1, SSBP4, LSM4, CDK4, and SMARCB1.

In embodiments, altering the gene signature comprises decreased expression and/or decreased activity in the progenitor cell of one or more genes selected from Table 5. In embodiments, the one or more genes selected from Table 5 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, or 49 genes selected from Table 5. In embodiments, the one or more genes selected from Table 5 comprises at least one of MYB, NCOA4, CEBPD, CEBPA, TLE4, DACH1, SMARCA2, GFI1, MAZ, TLE1, ELL2, and ARID1A. In embodiments, the one or more genes selected from Table 5 comprises at least one of MYB, NCOA4, CEBPD, CEBPA, and TLE4.

In embodiments, an increase in gene expression (e.g., the amount of mRNA expressed) may be about a 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about a 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more decrease in gene expression relative to a cell that has not been contacted with a perturbagen and/or relative to a cell that has been contacted with a no treatment control (including DMSO).

In various embodiments, an increase in gene expression (e.g., the amount of mRNA expressed) may be about a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater decrease in gene expression relative to a cell that has not been contacted with a perturbagen and/or relative to a cell that has been contacted with a no treatment control (including DMSO).

In embodiments, contacting the population of cells comprising a progenitor cell occurs in vitro or ex vivo.

In embodiments, contacting the population of cells comprising a progenitor cell occurs in vivo in a subject. In embodiments, the subject is a human. In embodiments, the human is an adult human.

In an aspect, the present disclosure provides a method for inhibiting the formation of a neutrophil, monocyte or immediate progenitor thereof. The method comprising a step of exposing a starting population of stem/progenitor cells comprising a non-lineage committed CD34+ cell to a perturbagen which prevents progression of a progenitor cell into and/or or reduces the likelihood that a progenitor cell will progress into a granulocyte monocyte progenitor, neutrophil progenitor, monocyte progenitor or other lineage-associated progenitor thereof. Embodiments associated with the above aspect are likewise relevant to the present aspect. In other words, each of the embodiments mentioned above for the above aspect may be revised/adapted to be applicable to the present aspect.

In an aspect, the present disclosure provides a method for inhibiting the formation of a neutrophil, monocyte or immediate progenitor thereof. The method comprising a step of exposing a starting population of stem/progenitor cells comprising a non-lineage committed CD34+ cell to a perturbation having a perturbation signature that prevents progression of a progenitor cell into and/or or reduces the likelihood that a progenitor cell will progress into a granulocyte monocyte progenitor, neutrophil progenitor, monocyte progenitor or other lineage-associated progenitor thereof. In this aspect, the perturbation signature comprises increased expression and/or activity of one or more of genes selected from Table 4 and/or a decreased expression and/or activity in the non-lineage committed CD34+ cell of one or more genes selected from Table 5. Embodiments associated with the above aspects are likewise relevant to the present aspect. In other words, each of the embodiments mentioned above for the above aspects may be revised/adapted to be applicable to the present aspect.

In another aspect, the present disclosure provides a method of reducing the quantity of neutrophils, monocytes or immediate progenitors thereof in a population of cells. The method comprising a step of exposing a starting population of stem/progenitor cells comprising a non-lineage committed CD34+ cell to a pharmaceutical composition that prevents progression of a progenitor cell into and/or reduces the likelihood that a progenitor cell will progress into a lineage specific progenitor population selected from granulocyte monocyte progenitor, neutrophil progenitor, monocyte progenitor, or other lineage-associated progenitors. The pharmaceutical composition inhibits the transition of a primitive stem/progenitor population into the lineage specific progenitor population that naturally would have the capacity to differentiate into neutrophils, monocytes or immediate progenitors thereof. In this aspect, the pharmaceutical composition comprises at least one perturbagen selected from Table 6, or a variant thereof. Embodiments associated with the above aspects are likewise relevant to the present aspect. In other words, each of the embodiments mentioned above for the above aspects may be revised/adapted to be applicable to the present aspect.

In yet another aspect, the present disclosure provides a perturbagen for use in any herein disclosed method. In a further aspect, the present disclosure provides a pharmaceutical composition comprising perturbagen for use in any herein disclosed method.

### Methods and Perturbagens for Treating a Disease or Disorder

The ability of a perturbagen to specifically prevent progression of a progenitor cell into and/or reduce the likelihood that a progenitor cell will progress into neutrophil and/or monocyte lineages (including the GMP lineage) would be valuable in designing a therapeutic composition. As examples, for a disease characterized by an increased number of neutrophils, a therapeutic composition comprising a perturbagen that decreases the number of neutrophils could be beneficial and/or a disease (including the same disease) that would benefit from decreased numbers of monocytes could be treated by a therapeutic composition comprising a perturbagen that decreases the number of monocytes.

An aspect of the present disclosure is a method for treating a disease or disorder characterized by an abnormal ratio of neutrophils to monocytes. The method comprising a step of: (a) administering to a patient in need thereof a therapeutically effective amount of at least one perturbagen selected from Table 6, or a variant thereof, in which the at least one perturbagen is capable of changing a gene signature in a progenitor cell; or (b) administering to a patient in need thereof a cell, the cell having been contacted with at least one perturbagen selected from Table 6, or a variant thereof, in which the at least one perturbagen is capable of changing a gene signature in a progenitor cell.

An aspect of the present disclosure is a method for treating a disease or disorder characterized by an abnormal ratio of neutrophils to monocytes. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

In embodiments, the abnormal ratio comprises an increased number of neutrophils.

In embodiments, the abnormal ratio comprises an increased number of monocytes.

In embodiments, the abnormal ratio comprises an increased number of neutrophils and an increased number of monocytes.

In embodiments, the abnormal ratio comprises an increased number of neutrophils and/or an increased number of monocytes. In embodiments, the abnormal ratio comprises an increased number of neutrophils. In embodiments, the abnormal ratio comprises an increased number of monocytes.

In embodiments, the administering is directed to the bone marrow of the patient. In embodiments, the administering is via intraosseous injection or intraosseous infusion.

In embodiments, the administering the cell is via intravenous injection or intravenous infusion.

In embodiments, the administering is simultaneously or sequentially to one or more mobilization agents.

In embodiments, the disease or disorder characterized by an abnormal ratio of neutrophils to monocytes is selected from leukocytosis, granulocytosis, monocytosis, neutrophilia, leukemia (e.g., Chronic myeloid leukemia (CML), Chronic myelomonocytic leukemia (CMML), and Acute myeloblastic leukemia (AML), including AML-M5), cancer induced leukocytosis, drug induced leukocytosis, infection induce leukocytosis, lupus (and other autoimmune disorders showing leukocytosis), and myeloproliferative neoplasms (including polycythemia vera and myelofibrosis).

In embodiments, the disease or disorder characterized by an abnormal ratio of neutrophils to monocytes is related to an autoimmune disease, a bone-marrow transplant, a cancer, a genetic disease, a bacterial infection, a viral infection, inflammatory bowel disease, tissue necrosis (e.g., myocardial infarction and burns), acute or chronic inflammation, a previous or concurrent anti-cancer treatment, and/or a previous or concurrent immune suppressive treatment.

Another aspect of the present disclosure is a method for treating a disease or disorder characterized by an abnormal ratio of neutrophils and/or monocytes to progenitor cells. The method comprising a step of: (a) administering to a patient in need thereof at least one perturbagen selected from Table 6, or a variant thereof, in which the at least one perturbagen is capable of changing a gene signature in a progenitor cell; or (b) administering to a patient in need thereof a cell, the cell having been contacted with at least one perturbagen selected from Table 6, or a variant thereof, in which the at least one perturbagen is capable of changing a gene signature in a progenitor cell.

Another aspect of the present disclosure is a method for treating a disease or disorder characterized by an abnormal ratio of neutrophils and/or monocytes to progenitor cells. The method comprising a step of: (a) administering to a patient in need thereof at least one perturbagen selected from Table 6, or a variant thereof; or (b) administering to a patient in need thereof a cell, the cell having been contacted with at least one perturbagen selected from Table 6, or a variant thereof.

In embodiments, the abnormal ratio comprises an increased number of neutrophils and/or an increased number of monocytes and/or a decreased number of progenitor cells. In embodiments, the abnormal ratio comprises an increased number of neutrophils. In embodiments, the abnormal ratio comprises an increased number of monocytes. In embodiments, the abnormal ratio comprises an increased number of neutrophils and an increased number of monocytes. In embodiments, the abnormal ratio comprises a decreased number of progenitor cells.

In embodiments, the administering is directed to the bone marrow of the patient. In embodiments, the administering is via intraosseous injection or intraosseous infusion.

In embodiments, the administering the cell is via intravenous injection or intravenous infusion. In embodiments, the administering is simultaneously or sequentially to one or more mobilization agents.

In embodiments, the disease or disorder characterized by an abnormal ratio of neutrophils and/or monocytes to progenitor cells is selected from leukocytosis, granulocytosis, monocytosis, neutrophilia, leukemia (e.g., Chronic myeloid leukemia (CML), Chronic myelomonocytic leukemia (CMML), and Acute myeloblastic leukemia (AML), including AML-M5), cancer induced leukocytosis, drug induced leukocytosis, infection induce leukocytosis, lupus (and other autoimmune disorders showing leukocytosis), and myeloproliferative neoplasms (including polycythemia vera and myelofibrosis).

In embodiments, the disease or disorder characterized by an abnormal ratio of neutrophils and/or monocytes to progenitor cells is related to an autoimmune disease, a bone-marrow transplant, a cancer, a genetic disease, a bacterial infection, a viral infection, inflammatory bowel disease, tissue necrosis (e.g., myocardial infarction and burns), acute or chronic inflammation, a previous or concurrent anti-cancer treatment, and/or, a previous or concurrent immune suppressive treatment.

In embodiments, an abnormal ratio (e.g., of neutrophils to monocytes, neutrophils to progenitors, and monocytes to progenitors) is relative to the normal, healthy state. As examples, diseases that are characterized by an increased amount/number of neutrophils (e.g., neutrophilia), diseases that are characterized by a reduced amount/number of neutrophils (e.g., neutropenia), diseases that are characterized by a reduced amount/number of monocytes (e.g., monocytopenia), and diseases that are characterized by an increased amount/number of monocytes (e.g., monocytosis). Normal ratios of cell types in the blood is well known in the art.

In an aspect, the present disclosure provides a method for treating monocytosis. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

In yet another aspect, the present disclosure provides a method for treating neutrophilia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

In yet another aspect, the present disclosure provides a method for treating a myeloproliferative neoplasm. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

An aspect of the present disclosure is a method for treating granulocytosis. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof. In embodiments, the granulocytosis is monocytosis. In embodiments, the granulocytosis is neutrophilia.

In embodiments, the at least one perturbagen is capable of changing a gene signature in a progenitor cell.

In embodiments, the therapeutically effective amount of the at least one perturbagen provides a change in a gene signature in a cell. In this embodiment, the change in the gene signature comprises an increase in expression and/or activity in the cell of one or more genes selected from Table 4 and/or a decrease in expression and/or activity in the cell of one or more genes selected from Table 5.

In embodiments, the therapeutically effective amount of the at least one perturbagen provides a decrease in the number of neutrophils and/or the number of monocytes in a sample from the human subject relative to the number of neutrophils and/or the number of monocytes in a sample from the human subject prior to administering the at least one perturbagen.

In embodiments, the therapeutically effective amount of the at least one perturbagen provides a decrease in the ratio of the number of neutrophils to the number of monocytes in a sample from the human subject relative to the ratio in a sample from the human subject prior to administering the at least one perturbagen.

In embodiments, the therapeutically effective amount of the at least one perturbagen provides a decrease in the ratio of the number of monocytes to the number of neutrophils in a sample from the human subject relative to the ratio in a sample from the human subject prior to administering the at least one perturbagen.

In embodiments, the therapeutically effective amount of the at least one perturbagen provides a decrease in the ratio of the number of neutrophils and/or the number of monocytes to the number of progenitor cells in a sample from the human subject relative to the ratio in a sample from the human subject prior to administering the at least one perturbagen.

In embodiments, the administering the therapeutically effective amount of the at least one perturbagen is directed to the bone marrow of the patient. In embodiments, the administering is via intraosseous injection or intraosseous infusion.

In embodiments, the administering the cell is via intravenous injection or intravenous infusion.

In embodiments, the administering is simultaneously or sequentially to one or more mobilization agents.

In embodiments, the administering occurs about once per day for one or more days.

In embodiments, the administering occurs more than once per day for one or more days.

In embodiments, the administering occurs at most once per day for one or more days.

In embodiments, the administering occurs substantially continuously per administration period.

In embodiments, the patient was selected by steps of, at least, obtaining from a subject having a disease or disorder a sample of cells comprising a non-lineage committed CD34+ cell and contacting the sample of cells with least one perturbagen selected from Table 6, or a variant thereof and which is capable of increasing the number of monocytes and/or neutrophils or a progenitor thereof in a culture from the sample of cells.

In embodiments, the patient was selected by steps of, at least, obtaining from a subject having a disease or disorder a sample of cells comprising a non-lineage committed CD34+ cell and contacting the sample of cells with least one perturbagen selected from Table 6, or a variant thereof. In this embodiment, the at least one perturbagen alters a gene signature in the sample of cells.

In embodiments, the patient was selected by steps of, at least, obtaining from a subject having a disease or disorder a sample of cells comprising a non-lineage committed CD34+ cell and contacting the sample of cells with at least one perturbagen capable of altering a gene signature in a non-lineage committed CD34+ cell. In this embodiment, the at least one perturbagen increases in the sample of cells the expression and/or activity of one or more genes selected from Table 4 and/or decreases in the sample of cells the expression and/or activity of one or more genes selected from Table 5.

In embodiments, the patient was selected by steps of, at least, obtaining from a subject having a disease or disorder a sample of cells comprising a non-lineage committed CD34+ cell and contacting the sample of cells with at least one perturbagen selected from Table 6, or a variant thereof. In this embodiment, the at least one perturbagen increases in the sample of cells the expression and/or activity of one or more genes selected from Table 4 and/or decreases in the sample of cells the expression and/or activity of one or more genes selected from Table 5.

### Administration, Dosing, and Treatment Regimens

As examples, administration results in the delivery of one or more perturbagens disclosed herein into the bloodstream (via enteral or parenteral administration), or alternatively, the one or more perturbagens is administered directly to the site of hematopoietic cell proliferation and/or maturation, i.e., in the bone marrow.

Delivery of one or more perturbagens disclosed herein to the bone marrow may be via intravenous injection or intravenous infusion or via intraosseous injection or intraosseous infusion. Devices and apparatuses for performing these delivery methods are well known in the art.

Delivery of one or more perturbagens disclosed herein into the bloodstream via intravenous injection or intravenous infusion may follow or be contemporaneous with stem cell mobilization. In stem cell mobilization, certain drugs are used to cause the movement of stem cells from the bone marrow into the bloodstream. Once in the bloodstream, the stem cells are contacted with the one or more perturbagens and are able to alter the stage of and/or a gene signature in a progenitor cell, for example. Drugs and methods relevant to stem cell mobilization are well known in the art; see, e.g., Mohammadi et al, “Optimizing Stem Cells Mobilization Strategies to Ameliorate Patient Outcomes: A Review of Guide-lines and Recommendations.” Int J Hematol Oncol Stem Cell Res. 2017 Jan. 1; 11 (1): 78-88; Hopman and DiPersio “Advances in Stem Cell Mobilization.” Blood Review, 2014, 28 (1): 31-40; and Kim “Hematopoietic stem cell mobilization: current status and future perspective.” Blood Res. 2017 June; 52 (2): 79-81. The contents of each of which is incorporated herein by reference in its entirety.

Dosage forms suitable for parenteral administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any perturbagen disclosed herein as well as the dosing schedule can depend on various parameters and factors, including, but not limited to, the specific perturbagen, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In another embodiment, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A perturbagen disclosed herein can be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled—or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled—or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, e.g., the bone marrow, thus requiring only a fraction of the systemic dose (see, e.g., Goodson, in Medical Applications of Controlled Release, supra, vol. 2, pp. 115-138 (1984). Other controlled-release systems discussed in the review by Langer, 1990, Science 249:1527-1533) may be used.

The dosage regimen utilizing any perturbagen disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed. Any perturbagen disclosed herein can be administered in a single daily dose, or the total daily dosage can be administered in divided doses of two, three or four times daily. Furthermore, any perturbagen disclosed herein can be administered continuously rather than intermittently throughout the dosage regimen.

Pharmaceutical compositions and Formulations

Aspects of the present disclosure include a pharmaceutical composition comprising a therapeutically effective amount of one or more perturbagens, as disclosed herein.

The perturbagens disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. In embodiments, the compositions disclosed herein are in the form of a pharmaceutically acceptable salt.

Further, any perturbagen disclosed herein can be administered to a subject as a component of a composition, e.g., pharmaceutical composition that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any perturbagen disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference. In embodiments, the compositions, e.g., pharmaceutical compositions, disclosed herein are suspended in a saline buffer (including, without limitation TBS, PBS, and the like).

The present disclosure includes the disclosed perturbagens in various formulations of pharmaceutical compositions. Any perturbagens disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use.

Where necessary, the pharmaceutical compositions comprising the perturbagens can also include a solubilizing agent. Also, the agents can be delivered with a suitable vehicle or delivery device as known in the art.

Combination therapies, comprising more than one perturbagen, can be co-delivered in a single delivery vehicle or delivery device.

Compositions for administration can optionally include a local anesthetic such as, for example, lignocaine to lessen pain at the site of the injection.

The pharmaceutical compositions comprising the perturbagens of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any perturbagens disclosed herein is formulated in accordance with routine procedures as a pharmaceutical composition adapted for a mode of administration disclosed herein.

### Other Aspects of the Present Disclosure

Embodiments associated with any of the above-disclosed aspects are likewise relevant to the below-mentioned aspects. In other words, each of the embodiments mentioned above for the above aspects may be revised/adapted to be applicable to the below aspects.

Yet another aspect of the present disclosure is a use of the perturbagen of Table 6, or a variant thereof in the manufacture of a medicament for treating a disease or disorder characterized by an abnormal ratio of neutrophils to monocytes.

In an aspect, the present disclosure provides a use of the perturbagen of Table 6, or a variant thereof in the manufacture of a medicament for treating a disease or disorder characterized by an abnormal ratio of neutrophils and/or monocytes to progenitor cells.

An aspect of the present disclosure is a method for selecting a patient for treatment. The method comprising steps of obtaining from a subject having a disease or disorder a sample of cells comprising a non-lineage committed CD34+ cell and contacting the sample of cells with least one perturbagen selected from Table 6, or a variant thereof. In this aspect, when the at least one perturbagen increases the number of monocytes and/or neutrophils or a progenitor thereof in a culture from the sample of cells, the subject is selected as a patient.

In another aspect, the present disclosure provides a method of identifying a perturbagen. The method comprising: exposing a starting population of progenitor cells, which naturally would have the capacity to undergo a change in cell state comprising differentiating into neutrophils, monocytes and/or immediate progenitors thereof, to a candidate perturbagen; determining an inhibition in the natural capacity to differentiate into neutrophils, monocytes and/or immediate progenitors thereof following exposure of the population of cells to the perturbagen, wherein the candidate perturbagen capable of inhibiting the natural capacity of a progenitor cell to differentiate into a neutrophil, monocyte and/or immediate progenitor thereof is identified as a perturbagen.

In another aspect, the present disclosure provides a method for making a therapeutic agent for a disease or disorder selected from leukocytosis, granulocytosis, monocytosis, neutrophilia, leukemia, cancer induced leukocytosis, drug induced leukocytosis, infection induce leukocytosis, lupus (and other autoimmune disorders showing leukocytosis), and myeloproliferative neoplasms (including polycythemia vera and myelofibrosis). The method comprising: exposing a starting population of progenitor cells, which naturally would have the capacity to undergo a change in cell state comprising differentiating into neutrophils, monocytes and/or immediate progenitors thereof, to a candidate perturbagen; determining an inhibition in the natural capacity to differentiate into neutrophils, monocytes and/or immediate progenitors thereof following exposure of the population of cells to the perturbagen, wherein the candidate perturbagen capable of inhibiting the natural capacity of a progenitor cell to differentiate into a neutrophil, monocyte and/or immediate progenitor thereof is identified as a perturbagen.

An aspect of the present disclosure is a method for selecting a patient for treatment. The method comprising steps of obtaining from a subject having a disease or disorder a sample of cells comprising a non-lineage committed CD34+ cell and contacting the sample of cells with least one perturbagen selected from Table 6, or a variant thereof. In this aspect, when the at least one perturbagen alters a gene signature in the sample of cells, the subject is selected as a patient.

Another aspect of the present disclosure is a method for selecting a patient for treatment. The method comprising steps of obtaining from a subject having a disease or disorder a sample of cells comprising a non-lineage committed CD34+ cell and contacting the sample of cells with at least one perturbagen capable of altering a gene signature in a non-lineage committed CD34+ cell. In this aspect, when the at least one perturbagen increases in the sample of cells the expression and/or activity of one or more genes selected from Table 4 and/or decreases in the sample of cells the expression and/or activity of one or more genes selected from Table 5, the subject is selected as a patient.

Yet another aspect of the present disclosure is a method for selecting a patient for treatment. The method comprising steps of obtaining from a subject having a disease or disorder a sample of cells comprising a non-lineage committed CD34+ cell and contacting the sample of cells with at least one perturbagen selected from Table 6, or a variant thereof. In this aspect, when the at least one perturbagen increases in the sample of cells the expression and/or activity of one or more genes selected from Table 4 and/or decreases in the sample of cells the expression and/or activity of one or more genes selected from Table 5, the subject is selected as a patient.

In another aspect, the present disclosure provides a method of identifying a candidate perturbation for inhibiting the transition of a progenitor cell into a neutrophil, monocyte or immediate progenitor thereof. The method comprising steps of: exposing a starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and preventing progression of a progenitor cell into and/or reducing the likelihood that a progenitor cell will progress into a neutrophil, monocyte or immediate progenitor thereof following exposure of the population of cells to the perturbation; and identifying the perturbation as a candidate perturbation for inhibiting the transition of a progenitor cell into a neutrophil, monocyte or immediate progenitor thereof based on the perturbation signature. In this aspect, the perturbation signature is an increase in expression and/or activity in the progenitor cell of one or more genes selected from Table 4, and/or a decrease in expression and/or activity in the progenitor cell of one or more genes selected from Table 5.

In another aspect, the present disclosure provides a method for making a therapeutic agent for a disease or disorder selected from leukocytosis, granulocytosis, monocytosis, neutrophilia, leukemia, cancer induced leukocytosis, drug induced leukocytosis, infection induced leukocytosis, lupus (and other autoimmune disorders showing leukocytosis), and myeloproliferative neoplasms (including polycythemia vera and myelofibrosis). The method comprises steps of: (a) identifying a therapeutic agent for therapy; and (b) formulating the therapeutic agent for the treatment of the disease or disorder. In this aspect, identifying a therapeutic agent for therapy comprises steps of: exposing a starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and preventing progression of a progenitor cell into and/or reducing the likelihood that a progenitor cell will progress into a neutrophil, monocyte or immediate progenitor thereof following exposure of the population of cells to the perturbation; and identifying the perturbation as a candidate perturbation for inhibiting the transition of a progenitor cell into a neutrophil, monocyte or immediate progenitor thereof based on the perturbation signature. Further, in this aspect, the perturbation signature is an increase in expression and/or activity in the progenitor cell of one or more genes selected from Table 4, and/or a decrease in expression and/or activity in the progenitor cell of one or more genes selected from Table 5.

In a further aspect, the present disclosure provides a method for obtaining a gene signature that defines genes relevant to preventing progression of a progenitor cell into and/or or reducing the likelihood that a progenitor cell will progress towards the granulocyte monocyte progenitor (GMP) lineage or within the GMP lineage.

An aspect of the present disclosure is a method for obtaining a gene signature capable of preventing progression of a progenitor cell into and/or or reducing the likelihood that a progenitor cell will progress towards the granulocyte monocyte progenitor (GMP) lineage or within the GMP lineage.

Another aspect of the present disclosure is a use of gene signature to identify a perturbagen capable of preventing progression of a progenitor cell into and/or or reducing the likelihood that a progenitor cell will progress towards the granulocyte monocyte progenitor (GMP) lineage or within the GMP lineage.

Yet another aspect of the present disclosure is a perturbagen capable of causing a change in a gene signature.

In an aspect, the present disclosure provides a perturbagen capable of inhibiting a change in cell fate.

In another aspect, the present disclosure provides a perturbagen capable of causing a change in a gene signature and inhibiting a change in cell fate.

In yet another aspect, the present disclosure provides a pharmaceutical composition comprising any herein disclosed perturbagen.

In a further aspect, the present disclosure provides a unit dosage form comprising an effective amount of the pharmaceutical composition comprising any herein disclosed perturbagen.

The instant disclosure also provides certain embodiments as follows: Embodiment 1001. A method for inhibiting a change in cell state of a progenitor cell comprising: contacting a population of cells comprising a progenitor cell with at least one perturbagen selected from Table 6, or a variant thereof, wherein the at least one perturbagen is capable of inhibiting a gene signature in the progenitor cell; and wherein the progenitor cell is a non-lineage committed CD34+ cell.
Embodiment 1002. A method for inhibiting a change in cell state of a progenitor cell, comprising: contacting a population of cells comprising a progenitor cell with at least one perturbagen capable of inhibiting a gene signature in the progenitor cell, wherein altering the gene signature comprises an increase in expression and/or activity in the progenitor cell of one or more genes selected from Table 4 and/or a decrease in expression and/or activity in the progenitor cell of one or more genes selected from Table 5 and wherein the progenitor cell is a non-lineage committed CD34+ cell.
Embodiment 1003. A method for inhibiting a change in cell state of a progenitor cell, comprising: contacting a population of cells comprising a progenitor cell with at least one perturbagen selected from Table 6, or a variant thereof, and capable of altering a gene signature in the progenitor cell, wherein altering the gene signature comprises an increase in expression and/or activity in the progenitor cell of one or more genes selected from Table 4 and/or a decrease in expression and/or activity in the progenitor cell of one or more genes selected from Table 5 and wherein the progenitor cell is a non-lineage committed CD34+ cell.
Embodiment 1004. The method of any one of Embodiments 1001 to 1003, wherein inhibiting the change in cell state provides a decrease in the number of neutrophils and/or the number of monocytes.
Embodiment 1005. The method of Embodiment 1004, wherein the decrease in the number of neutrophils and/or the number of monocytes is relative to the number of neutrophils and/or the number of monocytes obtained from a population of progenitor cells that is not contacted with the at least one perturbagen.
Embodiment 1006. The method of Embodiment 1004, wherein the decrease in the number of neutrophils and/or the number of monocytes is relative to the population of progenitor cells prior to contacting with the at least one perturbagen.
Embodiment 1007. The method of any one of Embodiments 1004 to 1006, wherein inhibiting the change in cell state provides a decrease in the number of neutrophils.
Embodiment 1008. The method of Embodiment 1007, wherein inhibiting the change in cell state provides a decrease in the number of monocytes.
Embodiment 1009. The method of Embodiment 1007, wherein inhibiting the change in cell state does not provide a substantial increase in the number of monocytes.
Embodiment 1010. The method of any one of Embodiments 1007 to 1009, wherein the ratio of the number of neutrophils to the number of monocytes is decreased relative to the ratio obtained from a population of progenitor cells that is not contacted with the at least one perturbagen.
Embodiment 1011. The method of any one of Embodiments 1007 to 1009, wherein the ratio of the number of neutrophils to the number of monocytes is decreased relative to the ratio in the population of progenitor cells prior to contacting with the at least one perturbagen.
Embodiment 1012. The method of any one of Embodiments 1004 to 1006, wherein inhibiting the change in cell state provides a decrease in the number of monocytes.
Embodiment 1013. The method of Embodiment 1012, wherein inhibiting the change in cell state provides a decrease in the number of neutrophils.
Embodiment 1014. The method of Embodiment 1012, wherein inhibiting the change in cell state does not provide a substantial increase in the number of neutrophils.
Embodiment 1015. The method of any one of Embodiments 1012 to 1014, wherein the ratio of the number of monocytes to the number of neutrophils is decreased relative to the ratio obtained from a population of progenitor cells that is not contacted with the at least one perturbagen.
Embodiment 1016. The method of any one of Embodiments 1012 to 1014, wherein the ratio of the number of monocytes to the number of neutrophils is decreased relative to the ratio in the population of progenitor cells prior to contacting with the at least one perturbagen.
Embodiment 1017. The method of Embodiment 1004, wherein the ratio of the number of neutrophils to the number of progenitor cells is decreased relative to the ratio obtained from a population of progenitor cells that is not contacted with the at least one perturbagen.
Embodiment 1018. The method of Embodiment 1004, wherein the ratio of the number of neutrophils to the number of progenitor cells is decreased relative to the ratio in the population of progenitor cells prior to contacting with the at least one perturbagen.
Embodiment 1019. The method of Embodiment 1004, wherein the ratio of the number of monocytes to the number of progenitor cells is decreased relative to the ratio obtained from a population of progenitor cells that is not contacted with the at least one perturbagen.
Embodiment 1020. The method of Embodiment 1004, wherein the ratio of the number of monocytes to the number of progenitor cells is decreased relative to the ratio in the population of progenitor cells prior to contacting with the at least one perturbagen.
Embodiment 1021. The method of any one of Embodiments 1004 to 1020, wherein the decrease in the number of neutrophils and/or the number of monocytes is due in part to decreased cell proliferation of the neutrophils and/or the monocytes.
Embodiment 1022. The method of any one of Embodiments 1004 to 1021, wherein the decrease in the number of neutrophils and/or the number of monocytes is due in part to a decreased lifespan of the neutrophils and/or the monocytes.
Embodiment 1023. The method of any one of Embodiments 1004 to 1022, wherein the decrease in the number of neutrophils and/or the number of monocytes is due in part to increased cell death among the neutrophils and/or the monocytes.
Embodiment 1024. The method of any one of Embodiments 1004 to 1023, wherein the decrease in the number of neutrophils and/or the number of monocytes is due in part to blocking the progression from progenitor cells into the neutrophil and/or monocyte lineage.
Embodiment 1025. The method of any one of Embodiments 1021 to 1024, wherein the number of progenitor cells is increased.
Embodiment 1026. The method of Embodiment 1025, wherein the increase in the number of progenitor cells is due in part to increased cell proliferation of the progenitor cells.
Embodiment 1027. The method of Embodiment 1025 or Embodiment 1026, wherein the increase in the number of progenitor cells is due in part to an increased lifespan of the progenitor cells.
Embodiment 1028. The method of any one of Embodiments 1025 to 1027, wherein the increase in the number of progenitor cells is due in part to decreased cell death among the progenitor cells.
Embodiment 1029. The method of any one of Embodiments 1025 to 1028, wherein the increase in the number of progenitor cells is relative to the number of progenitor cells in a population of progenitor cells that is not contacted with the at least one perturbagen.
Embodiment 1030. The method of any one of Embodiments 1025 to 1029, wherein the increase in the number of progenitor cells is relative to the number of progenitor cells in the population prior to contacting with the at least one perturbagen.
Embodiment 1031. The method of any one of Embodiments 1025 to 1030, wherein the increase in the number of progenitor cells is due to inhibiting a change of cell state from a progenitor cell into the neutrophil lineage and/or monocyte lineage.
Embodiment 1032. The method of any one of Embodiments 1021 to 1024, wherein the number of CD66b+neutrophil progenitors, CD64+monocytic progenitors, neutrophilic promyelocytes, neutrophilic myelocytes, neutrophilic metamyelocytes, and/or neutrophilic band cells is decreased and/or the number of CD34+CD38+/− cells is decreased about two days after contacting the population of cells comprising a CD34+ cell with the at least one perturbagen and/or the number of CD34-CD38+/− cells is decreased about five days after contacting the population of cells comprising a CD34+ cell with the at least one perturbagen.
Embodiment 1033. The method of Embodiment 1032, wherein the ratio of the number of CD66b+ neutrophil progenitors, CD64+monocytic progenitors, neutrophilic promyelocytes, neutrophilic myelocytes, neutrophilic metamyelocytes, neutrophilic band cells, CD34+CD38+/− cells, and/or CD34-CD38+/− cells to the number of progenitor cells is decreased relative to the ratio obtained from a population of progenitor cells that is not contacted with the at least one perturbagen.
Embodiment 1034. The method of Embodiment 1032, wherein the ratio of the number of CD66b+ neutrophil progenitors, CD64+monocytic progenitors, neutrophilic promyelocytes, neutrophilic myelocytes, neutrophilic cells is decreased relative to the ratio in the population of progenitor cells prior to contacting with the at least one perturbagen.
Embodiment 1035. The method of any one of Embodiments 1021 to 1024, wherein the number of monoblasts and/or promonocytes is decreased.
Embodiment 1036. The method of Embodiment 1035, wherein the ratio of the number of monoblasts and/or promonocytes to the number of progenitor cells is decreased relative to the ratio obtained from a population of progenitor cells that is not contacted with the at least one perturbagen.
Embodiment 1037. The method of Embodiment 1035, wherein the ratio of the number of monoblasts and/or promonocytes to the number of progenitor cells is decreased relative to the ratio in the population of progenitor cells prior to contacting with the at least one perturbagen.
Embodiment 1038. The method of any one of Embodiments 1021 to 1024, wherein the number of granulocyte-monocyte progenitor (GMP) cells is decreased.
Embodiment 1039. The method of Embodiment 1038, wherein the ratio of the number of GMP cells to the number of progenitor cells for the GMP cell is decreased relative to the ratio obtained from a population of progenitor cells that is not contacted with the at least one perturbagen.
Embodiment 1040. The method of Embodiment 1038, wherein the ratio of the number of GMP cells to the number of progenitor cells for the GMP cell is decreased relative to the ratio in the population of progenitor cells prior to contacting with the at least one perturbagen.
Embodiment 1041. The method of Embodiment 1040, wherein the progenitor cell for the GMP cell is a common myeloid progenitor (CMP).
Embodiment 1042. The method of Embodiment 1038, wherein the number of monocyte-dendritic cell progenitor (MDP) cells is increased.
Embodiment 1043. The method of Embodiment 1042, wherein the ratio of the number of GMP cells to the number of MDP cells is decreased relative to the ratio obtained from a population of progenitor cells that is not contacted with the at least one perturbagen.
Embodiment 1044. The method of Embodiment 1042, wherein the ratio of the number of GMP cells to the number of MDP cells is decreased relative to the ratio in the population of progenitor cells prior to contacting with the at least one perturbagen.
Embodiment 1045. The method of any one of Embodiments 1001 to 1044, wherein the at least one perturbagen selected from Table 6, or a variant thereof, comprises at least 2, at least 3, at least 4, at least 5, at least 6, at least 7, at least 8, at least 9, or 10 perturbagens selected from Table 6, or variants thereof.
Embodiment 1046. The method of Embodiment 1045, wherein the at least one prevents progression of a progenitor cell into and/or reduces the likelihood that a progenitor cell will progress into the monocyte lineage. Embodiment 1047. The method of Embodiment 1045, wherein the at least one perturbagen prevents progression of a progenitor cell into and/or reduces the likelihood that a progenitor cell will progress into the neutrophil lineage.
Embodiment 1048. The method of any one of Embodiments 1001 to 1047, wherein altering the gene signature comprises increased expression and/or increased activity in the progenitor cell of one or more genes selected from Table 4.
Embodiment 1049. The method of Embodiment 1048, wherein the one or more genes selected from Table 4 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, or 72 genes selected from Table 4.
Embodiment 1050. The method of Embodiment 1046 or Embodiment 1049, wherein the one or more genes selected from Table 4 comprises at least one of HMGA1, SSBP4, LSM4, CDK4, SMARCB1, AES, GTF3A, RAB8A, RNPS1, THRAP3, and SNRPB.
Embodiment 1051. The method of Embodiment 1050, wherein the one or more genes selected from Table 4 comprises at least one of HMGA1, SSBP4, LSM4, CDK4, and SMARCB1.
Embodiment 1052. The method of any one of Embodiments 1001 to 1051, wherein altering the gene signature comprises decreased expression and/or decreased activity in the progenitor cell of one or more genes selected from Table 5.
Embodiment 1053. The method of Embodiment 1052, wherein the one or more genes selected from Table 5 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, or 49 genes selected from Table 5.
Embodiment 1054. The method of Embodiment 1052 or Embodiment 1053, wherein the one or more genes selected from Table 5 comprises at least one of MYB, NCOA4, CEBPD, CEBPA, TLE4, DACH1, SMARCA2, GFI1, MAZ, TLE1, ELL2, and ARID1A.
Embodiment 1055. The method of Embodiment 1054, wherein the one or more genes selected from Table 5 comprises at least one of MYB, NCOA4, CEBPD, CEBPA, and TLE4.
Embodiment 1056. The method of any one of Embodiments 1001 to 1055, wherein contacting the population of cells comprising a progenitor cell occurs in vitro or ex vivo.
Embodiment 1057. The method of any one of Embodiments 1001 to 1055, wherein contacting the population of cells comprising a progenitor cell occurs in vivo in a subject.
Embodiment 1058. The method of Embodiment 1057, wherein the subject is a human.
Embodiment 1059. The method of Embodiment 1058, wherein the human is an adult human.
Embodiment 1060. A perturbagen for use in the method of any one of Embodiments 1001 to 1059.
Embodiment 1061. A pharmaceutical composition comprising the perturbagen of Embodiment 1060.
Embodiment 1062. A method for inhibiting the formation of a neutrophil, a monocyte, or an immediate progenitor thereof, comprising: exposing a starting population of stem/progenitor cells comprising a non-lineage committed CD34+ cell to a perturbation having a perturbation signature that prevents progression of a progenitor cell into and/or or reduces the likelihood that a progenitor cell will progress into a granulocyte monocyte progenitor, neutrophil progenitor, monocyte progenitor or other lineage associated progenitor thereof, wherein the perturbation signature comprises increased expression and/or activity of one or more of genes selected from Table 4 and/or a decreased expression and/or activity in the non-lineage committed CD34+ cell of one or more genes selected from Table 5.
Embodiment 1063. A method of reducing the quantity of neutrophils, monocytes, or immediate progenitors thereof in a population of cells, comprising: exposing a starting population of stem/progenitor cells comprising a non-lineage committed CD34+ cell to a pharmaceutical composition that prevents progression of a progenitor cell into and/or reduces the likelihood that a progenitor cell will progress into a lineage specific progenitor population selected from granulocyte monocyte progenitor, neutrophil progenitor, monocyte progenitor, or other lineage-associated progenitors, the pharmaceutical composition inhibiting the transition of a primitive stem/progenitor population into the lineage specific progenitor population that naturally would have the capacity to differentiate into neutrophils, monocytes or immediate progenitors thereof, wherein the pharmaceutical composition comprises at least one perturbagen selected from Table 6, or a variant thereof.
Embodiment 1064. A method for treating a disease or disorder characterized by an abnormal ratio of neutrophils to monocytes, comprising: (a) administering to a patient in need thereof a therapeutically effective amount of at least one perturbagen selected from Table 6, or a variant thereof, wherein the at least one perturbagen is capable of changing a gene signature in a progenitor cell or (b) administering to a patient in need thereof a cell, the cell having been contacted with at least one perturbagen selected from Table 6, or a variant thereof, wherein the at least one perturbagen is capable of changing a gene signature in a progenitor cell.
Embodiment 1065. The method of Embodiment 1064, wherein the abnormal ratio comprises an increased number of neutrophils and/or an increased number of monocytes.
Embodiment 1066. The method of Embodiment 1064, wherein the abnormal ratio comprises an increased number of neutrophils.
Embodiment 1067. The method of Embodiment 1064, wherein the abnormal ratio comprises an increased number of monocytes.
Embodiment 1068. The method of any one of Embodiments 1064 to 1067, wherein the administering is directed to the bone marrow of the patient.
Embodiment 1069. The method of Embodiment 1068, wherein the administering is via intraosseous injection or intraosseous infusion.
Embodiment 1070. The method of any one of Embodiments 1064 to 1067, wherein the administering the cell is via intravenous injection or intravenous infusion.
Embodiment 1071. The method of any one of Embodiments 1064 to 1070, wherein the administering is simultaneously or sequentially to one or more mobilization agents.
Embodiment 1072. The method of any one of Embodiments 1064 to 1071, wherein the disease or disorder characterized by an abnormal ratio of neutrophils to monocytes is selected from leukocytosis, granulocytosis, monocytosis, and neutrophilia, leukemia (e.g., Chronic myeloid leukemia (CML), Chronic myelomonocytic leukemia (CMML), and Acute myeloblastic leukemia (AML), including AML-M5), cancer induced leukocytosis, drug induced leukocytosis, infection induced leukocytosis, lupus (and other autoimmune disorders showing leukocytosis), and myeloproliferative neoplasms (including polycythemia vera and myelofibrosis).
Embodiment 1073. The method of any one of Embodiments 1064 to 1072, wherein the disease or disorder characterized by an abnormal ratio of neutrophils to monocytes is related to an autoimmune disease, a bone-marrow transplant, a cancer, a genetic disease, a bacterial infection, a viral infection, inflammatory bowel disease, tissue necrosis (e.g., myocardial infarction and burns), acute or chronic inflammation, a previous or concurrent anti-cancer treatment, and/or a previous or concurrent immune suppressive treatment.
Embodiment 1074. A method for treating a disease or disorder characterized by an abnormal ratio of neutrophils and/or monocytes to progenitor cells, comprising: (a) administering to a patient in need thereof at least one perturbagen selected from Table 6, or a variant thereof, wherein the at least one perturbagen is capable of changing a gene signature in a progenitor cell or (b) administering to a patient in need thereof a cell, the cell having been contacted with at least one perturbagen selected from Table 6, or a variant thereof, wherein the at least one perturbagen is capable of changing a gene signature in a progenitor cell.
Embodiment 1075. The method of Embodiment 1074, wherein the abnormal ratio comprises an increased number of neutrophils and/or an increased number of monocytes and/or a decreased number of progenitor cells.
Embodiment 1076. The method of Embodiment 1075, wherein the abnormal ratio comprises an increased number of neutrophils and an increased number of monocytes.
Embodiment 1077. The method of Embodiment 1075, wherein the abnormal ratio comprises an increased number of monocytes.
Embodiment 1078. The method of Embodiment 1075, wherein the abnormal ratio comprises an increased number of neutrophils.
Embodiment 1079. The method of any one of Embodiments 1076 to 1078, wherein the abnormal ratio comprises a decreased number of progenitor cells.
Embodiment 1080. The method of any one of Embodiments 1073 to 1079, wherein the administering is directed to the bone marrow of the patient.
Embodiment 1081. The method of Embodiment 1080, wherein the administering is via intraosseous injection or intraosseous infusion.
Embodiment 1082. The method of any one of Embodiments 1074 to 1079, wherein the administering the cell is via intravenous injection or intravenous infusion.
Embodiment 1083. The method of any one of Embodiments 1074 to 1082, wherein the administering is simultaneously or sequentially to one or more mobilization agents.
Embodiment 1084. The method of any one of Embodiments 1074 to 1083, wherein the disease or disorder characterized by an abnormal ratio of neutrophils and/or monocytes to progenitor cells is selected from leukocytosis, granulocytosis, monocytosis, and neutrophilia, leukemia (e.g., Chronic myeloid leukemia (CML), Chronic myelomonocytic leukemia (CMML), and Acute myeloblastic leukemia (AML), including AML-M5), cancer induced leukocytosis, drug induced leukocytosis, infection induced leukocytosis, lupus (and other autoimmune disorders showing leukocytosis), and myeloproliferative neoplasms (including polycythemia vera and myelofibrosis).
Embodiment 1085. The method of any one of Embodiments 1074 to 1084, wherein the disease or disorder characterized by an abnormal ratio of neutrophils and/or monocytes to progenitor cells is related to an autoimmune disease, a bone-marrow transplant, a cancer, a genetic disease, a bacterial infection, a viral infection, inflammatory bowel disease, tissue necrosis (e.g., myocardial infarction and burns), acute or chronic inflammation, a previous or concurrent anti-cancer treatment, and/or, a previous or concurrent immune suppressive treatment.
Embodiment 1086. A method for treating monocytosis, comprising: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.
Embodiment 1087. A method for treating neutrophilia, comprising: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.
Embodiment 1088. A method for treating a myeloproliferative neoplasm, comprising: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.
Embodiment 1089. The method of Embodiment 1088, wherein the myeloproliferative neoplasm is polycythemia vera.
Embodiment 1090. The method of Embodiment 1088, wherein the myeloproliferative neoplasm is myelofibrosis.
Embodiment 1091. A method for treating granulocytosis, comprising: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.
Embodiment 1092. The method of Embodiment 1091 wherein the granulocytosis is monocytosis.
Embodiment 1093. The method of Embodiment 1091 wherein the granulocytosis is neutrophilia.
Embodiment 1094. The method of any one of Embodiments 1086 to 1093, wherein the at least one perturbagen is capable of changing a gene signature in a progenitor cell.
Embodiment 1095. The method of any one of Embodiments 1086 to 1094, wherein the therapeutically effective amount of the at least one perturbagen provides a change in a gene signature in a cell, wherein the change in the gene signature comprises an increase in expression and/or activity in the cell of one or more genes selected from Table 4 and/or a decrease in expression and/or activity in the cell of one or more genes selected from Table 5.
Embodiment 1096. The method of any one of Embodiments 1086 to 1095, wherein the therapeutically effective amount of the at least one perturbagen provides a decrease in the number of neutrophils and/or the number of monocytes in a sample from the human subject relative to the number of neutrophils and/or the number of monocytes in a sample from the human subject prior to administering the at least one perturbagen.
Embodiment 1097. The method of any one of Embodiments 1086 to 1096, wherein the therapeutically effective amount of the at least one perturbagen provides a decrease in the ratio of the number of neutrophils to the number of monocytes in a sample from the human subject relative to the ratio in a sample from the human subject prior to administering the at least one perturbagen.
Embodiment 1098. The method of any one of Embodiments 1086 to 1096, wherein the therapeutically effective amount of the at least one perturbagen provides a decrease in the ratio of the number of monocytes to the number of neutrophils in a sample from the human subject relative to the ratio in a sample from the human subject prior to administering the at least one perturbagen.
Embodiment 1099. The method of any one of Embodiments 1086 to 1098, wherein the therapeutically effective amount of the at least one perturbagen provides a decrease in the ratio of the number of neutrophils and/or the number of monocytes to the number of progenitor cells in a sample from the human subject relative to the ratio in a sample from the human subject prior to administering the at least one perturbagen.
Embodiment 1100. The method of any one of Embodiments 1086 to 1099, wherein the administering the therapeutically effective amount of the at least one perturbagen is directed to the bone marrow of the patient.
Embodiment 1101. The method of Embodiment 1100, wherein the administering is via intraosseous injection or intraosseous infusion.
Embodiment 1102. The method of any one of Embodiments 1086 to 1099, wherein the administering the cell is via intravenous injection or intravenous infusion.
Embodiment 1103. The method of any one of Embodiments 1086 to 1099, wherein the administering is simultaneously or sequentially to one or more mobilization agents.
Embodiment 1104. The method of any one of Embodiments 1064 to 1099, wherein the administering occurs about once per day for one or more days.
Embodiment 1105. The method of any one of Embodiments 1064 to 1099, wherein the administering occurs more than once per day for one or more days.
Embodiment 1106. The method of any one of Embodiments 1064 to 1099, wherein the administering occurs at most once per day for one or more days.
Embodiment 1107. The method of any one of Embodiments 1064 to 1099, wherein the administering occurs substantially continuously per administration period.
Embodiment 1108. The method of any one of Embodiments 1064 to 1107, wherein the patient was selected by steps comprising: obtaining from the patient having the disease or disorder a sample of cells comprising a non-lineage committed CD34+ cell; and contacting the sample of cells with least one perturbagen selected from Table 6, or a variant thereof, wherein the at least one perturbagen alters a gene signature in the sample of cells.
Embodiment 1109. The method of any one of Embodiments 1064 to 1107, wherein the patient was selected by steps comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein the at least one perturbagen increases in the sample of cells the expression and/or activity of one or more genes selected from Table 4 and/or decreases in the sample of cells the expression and/or activity of one or more genes selected from Table 5.
Embodiment 1110. The method of any one of Embodiments 1064 to 1107, wherein the patient was selected by steps comprising: obtaining from a subject having the disease or disorder a sample of cells comprising a non-lineage committed CD34+ cell; and contacting the sample of cells with at least one perturbagen selected from Table 6, or a variant thereof; wherein the at least one perturbagen increases in the sample of cells the expression and/or activity of one or more genes selected from Table 4 and/or decreases in the sample of cells the expression and/or activity of one or more genes selected from Table 5.
Embodiment 1111. A method for selecting the patient of any one of Embodiments 1064 to 1107, comprising: obtaining from a subject having the disease or disorder a sample of cells comprising a non-lineage committed CD34+ cell; and contacting the sample of cells with least one perturbagen selected from Table 6, or a variant thereof, wherein when the at least one perturbagen alters a gene signature in the sample of cells, the subject is selected as a patient.
Embodiment 1112. A method for selecting the patient of any one of Embodiments 1064 to 1107, comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein when the at least one perturbagen increases in the sample of cells the expression and/or activity of one or more genes selected from Table 4 and/or decreases in the sample of cells the expression and/or activity of one or more genes selected from Table 5, the subject is selected as a patient. Embodiment 1113. A method for selecting the patient of any one of Embodiments 1064 to 1107, comprising: obtaining from a subject having the disease or disorder a sample of cells comprising a non-lineage committed CD34+ cell; and contacting the sample of cells with at least one perturbagen selected from Table 6, or a variant thereof; wherein when the at least one perturbagen increases in the sample of cells the expression and/or activity of one or more genes selected from Table 4 and/or decreases in the sample of cells the expression and/or activity of one or more genes selected from Table 5, the subject is selected as a patient.
Embodiment 1114. Use of the perturbagen of Table 6, or a variant thereof in the manufacture of a medicament for treating a disease or disorder characterized by an abnormal ratio of neutrophils to monocytes. Embodiment 1115. Use of the perturbagen of Table 6, or a variant thereof in the manufacture of a medicament for treating a disease or disorder characterized by an abnormal ratio of neutrophils and/or monocytes to progenitor cells.
Embodiment 1116. A method of identifying a candidate perturbation for inhibiting the transition of a progenitor cell into a neutrophil, monocyte or immediate progenitor thereof, the method comprising: exposing a starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and preventing progression of a progenitor cell into and/or reducing the likelihood that a progenitor cell will progress into a neutrophil, monocyte or immediate progenitor thereof following exposure of the population of progenitor cells to the perturbation; and identifying the perturbation as a candidate perturbation for inhibiting the transition of a progenitor cell into a neutrophil, monocyte or immediate progenitor thereof based on the perturbation signature, wherein the perturbation signature is an increase in expression and/or activity in the progenitor cell of one or more genes selected from Table 4, and/or a decrease in expression and/or activity in the progenitor cell of one or more genes selected from Table 5.
Embodiment 1117. A method for making a therapeutic agent for a disease or disorder selected from leukocytosis, granulocytosis, monocytosis, and neutrophilia, leukemia, cancer induced leukocytosis, drug induced leukocytosis, infection induce leukocytosis, lupus (and other autoimmune disorders showing leukocytosis), and myeloproliferative neoplasms (including polycythemia vera and myelofibrosis), comprising: (a) identifying a therapeutic agent for therapy according to the method of Embodiment 1116 and (b) formulating the therapeutic agent for the treatment of the disease or disorder.
Embodiment 3001. A method for inhibiting a change in cell state of a progenitor cell comprising: contacting a population of cells comprising a progenitor cell with at least one perturbagen selected from Table 6, or a variant thereof, wherein the at least one perturbagen is capable of inhibiting a change in cell state of a non-lineage committed CD34+progenitor cell and wherein the progenitor cell is a non-lineage committed CD34+ cell.
Embodiment 3002. The method of Embodiment 3001, wherein inhibiting the change in cell state provides a decrease in the number of neutrophils.
Embodiment 3003. The method of Embodiment 3001, wherein inhibiting the change in cell state provides a decrease in the number of monocytes.
Embodiment 3004. The method of any one of Embodiments 3001 to 3003, wherein inhibiting the change in cell state provides a decrease in the number of neutrophils and the number of monocytes.
Embodiment 3005. The method of Embodiment 3004, wherein the decrease in the number of neutrophils and/or the number of monocytes is relative to the number of neutrophils and/or the number of monocytes obtained from a population of progenitor cells that is not contacted with the at least one perturbagen.
Embodiment 3006. The method of Embodiment 3004, wherein the decrease in the number of neutrophils and/or the number of monocytes is relative to the population of progenitor cells prior to contacting with the at least one perturbagen.
Embodiment 3007. The method of any one of Embodiments 3001 to 3004, wherein inhibiting the change in cell state provides a decrease in the number of neutrophils.
Embodiment 3008. The method of Embodiment 3007, wherein inhibiting the change in cell state provides a decrease in the number of monocytes.
Embodiment 3009. The method of Embodiment 3007, wherein the inhibiting change in cell state does not provide a substantial increase in the number of monocytes.
Embodiment 3010. The method of any one of Embodiments 3007 to 3009, wherein the ratio of the number of neutrophils to the number of monocytes is decreased relative to the ratio obtained from a population of progenitor cells that is not contacted with the at least one perturbagen.
Embodiment 3011. The method of any one of Embodiments 3007 to 3009, wherein the ratio of the number of neutrophils to the number of monocytes is decreased relative to the ratio in the population of progenitor cells prior to contacting with the at least one perturbagen.
Embodiment 3012. The method of any one of Embodiments 3004 to 3011, wherein inhibiting the change in cell state provides a decrease in the number of monocytes.
Embodiment 3013. The method of Embodiment 3012, wherein inhibiting the change in cell state provides a decrease in the number of neutrophils.
Embodiment 3014. The method of Embodiment 3012, wherein inhibiting the change in cell state does not provide a substantial increase in the number of neutrophils.
Embodiment 3015. The method of any one of Embodiments 3012 to 3014, wherein the ratio of the number of monocytes to the number of neutrophils is decreased relative to the ratio obtained from a population of progenitor cells that is not contacted with the at least one perturbagen.
Embodiment 3016. The method of any one of Embodiments 3012 to 3014, wherein the ratio of the number of monocytes to the number of neutrophils is decreased relative to the ratio in the population of progenitor cells prior to contacting with the at least one perturbagen.
Embodiment 3017. The method of Embodiment 3004, wherein the ratio of the number of neutrophils to the number of progenitor cells is decreased relative to the ratio obtained from a population of progenitor cells that is not contacted with the at least one perturbagen.
Embodiment 3018. The method of Embodiment 3004, wherein the ratio of the number of neutrophils to the number of progenitor cells is decreased relative to the ratio in the population of progenitor cells prior to contacting with the at least one perturbagen.
Embodiment 3019. The method of Embodiment 3004, wherein the ratio of the number of monocytes to the number of progenitor cells is decreased relative to the ratio obtained from a population of progenitor cells that is not contacted with the at least one perturbagen.
Embodiment 3020. The method of Embodiment 3004, wherein the ratio of the number of monocytes to the number of progenitor cells is decreased relative to the ratio in the population of progenitor cells prior to contacting with the at least one perturbagen.
Embodiment 3021. The method of any one of Embodiments 3004 to 3020, wherein the decrease in the number of neutrophils and/or the number of monocytes is due in part to decreased cell proliferation of the neutrophils and/or the monocytes.
Embodiment 3022. The method of any one of Embodiments 3004 to 3021, wherein the decrease in the number of neutrophils and/or the number of monocytes is due in part to a decreased lifespan of the neutrophils and/or the monocytes.
Embodiment 3023. The method of any one of Embodiments 3004 to 3022, wherein the decrease in the number of neutrophils and/or the number of monocytes is due in part to increased cell death among the neutrophils and/or the monocytes.
Embodiment 3024. The method of any one of Embodiments 3004 to 3023, wherein the decrease in the number of neutrophils and/or the number of monocytes is due in part to blocking the progression from progenitor cells into the neutrophil and/or monocyte lineage.
Embodiment 3025. The method of any one of Embodiments 3021 to 3024, wherein the number of progenitor cells is increased.
Embodiment 3026. The method of Embodiment 3025, wherein the increase in the number of progenitor cells is due in part to increased cell proliferation of the progenitor cells.
Embodiment 3027. The method of Embodiment 3025 or Embodiment 3026, wherein the increase in the number of progenitor cells is due in part to an increased lifespan of the progenitor cells.
Embodiment 3028. The method of any one of Embodiments 3025 to 3027, wherein the increase in the number of progenitor cells is due in part to decreased cell death among the progenitor cells.
Embodiment 3029. The method of any one of Embodiments 3025 to 3028, wherein the increase in the number of progenitor cells is relative to the number of progenitor cells in a population of progenitor cells that is not contacted with the at least one perturbagen.
Embodiment 3030. The method of any one of Embodiments 3025 to 3029, wherein the increase in the number of progenitor cells is relative to the number of progenitor cells in the population prior to contacting with the at least one perturbagen.
Embodiment 3031. The method of any one of Embodiments Embodiment 3025 to 3030, wherein the increase in the number of progenitor cells is due to inhibiting the change of cell state from a progenitor cell into the neutrophil lineage and/or monocyte lineage.
Embodiment 3032. The method of any one of Embodiments 3021 to 3024, wherein the number of CD66b+neutrophil progenitors, CD64+monocytic progenitors, neutrophilic promyelocytes, neutrophilic myelocytes, neutrophilic metamyelocytes, and/or neutrophilic band cells is decreased and/or the number of CD34+CD38+/− cells is decreased about two days after contacting the population of cells comprising a CD34+ cell with the at least one perturbagen and/or the number of CD34-CD38+/− cells are decreased about five days after contacting the population of cells comprising a CD34+ cell with the at least one perturbagen.
Embodiment 3033. The method of Embodiment 3032, wherein the ratio of the number of CD66b+ neutrophil progenitors, CD64+monocytic progenitors, neutrophilic promyelocytes, neutrophilic myelocytes, neutrophilic metamyelocytes, neutrophilic band cells, CD34+CD38+/− cells, and/or CD34-CD38+/− cells to the number of progenitor cells is decreased relative to the ratio obtained from a population of progenitor cells that is not contacted with the at least one perturbagen.
Embodiment 3034. The method of Embodiment 3032, wherein the ratio of the number of CD66b+ neutrophil progenitors, CD64+monocytic progenitors, neutrophilic promyelocytes, neutrophilic myelocytes, neutrophilic cells is decreased relative to the ratio in the population of progenitor cells prior to contacting with the at least one perturbagen.
Embodiment 3035. The method of any one of Embodiments 3021 to 3024, wherein the number of monoblasts and/or promonocytes is decreased.
Embodiment 3036. The method of Embodiment 3035, wherein the ratio of the number of monoblasts and/or promonocytes to the number of progenitor cells is decreased relative to the ratio obtained from a population of progenitor cells that is not contacted with the at least one perturbagen.
Embodiment 3037. The method of Embodiment 3035, wherein the ratio of the number of monoblasts and/or promonocytes to the number of progenitor cells is decreased relative to the ratio in the population of progenitor cells prior to contacting with the at least one perturbagen.
Embodiment 3038. The method of any one of Embodiments 3021 to 3024, wherein the number of granulocyte-monocyte progenitor (GMP) cells is decreased.
Embodiment 3039. The method of Embodiment 3038, wherein the ratio of the number of GMP cells to the number of progenitor cells for the GMP cell is decreased relative to the ratio obtained from a population of progenitor cells that is not contacted with the at least one perturbagen.
Embodiment 3040. The method of Embodiment 3039, wherein the ratio of the number of GMP cells to the number of progenitor cells for the GMP cell is decreased relative to the ratio in the population of progenitor cells prior to contacting with the at least one perturbagen.
Embodiment 3041. The method of Embodiment 3040, wherein the progenitor cell for the GMP cell is a common myeloid progenitor (CMP).
Embodiment 3042. The method of Embodiment 3038, wherein the number of monocyte-dendritic cell progenitor (MDP) cells is increased.
Embodiment 3043. The method of Embodiment 3042, wherein the ratio of the number of GMP cells to the number of MDP cells is decreased relative to the ratio obtained from a population of progenitor cells that is not contacted with the at least one perturbagen.
Embodiment 3044. The method of Embodiment 3042, wherein the ratio of the number of GMP cells to the number of MDP cells is decreased relative to the ratio in the population of progenitor cells prior to contacting with the at least one perturbagen.
Embodiment 3045. The method of any one of Embodiments 3001 to 3044, wherein the at least one perturbagen selected from Table 6, or a variant thereof, comprises at least 2, at least 3, at least 4, at least 5, at least 6, at least 7, at least 8, at least 9, or 10 perturbagens selected from Table 6, or variants thereof.
Embodiment 3046. The method of Embodiment 3045, wherein the at least one perturbagen prevents progression of a progenitor cell into and/or reduces the likelihood that a progenitor cell will progress into the monocyte lineage.
Embodiment 3047. The method of Embodiment 3045, wherein the at least one perturbagen prevents progression of a progenitor cell into and/or reduces the likelihood that a progenitor cell will progress into the neutrophil lineage.
Embodiment 3048. The method of any one of Embodiments 3001 to 3047, wherein contacting the population of cells comprising a progenitor cell occurs in vitro or ex vivo.
Embodiment 3049. The method of any one of Embodiments 3001 to 3047, wherein contacting the population of cells comprising a progenitor cell occurs in vivo in a subject.
Embodiment 3050. The method of Embodiment 3049, wherein the subject is a human.
Embodiment 3051. The method of Embodiment 3050, wherein the human is an adult human.
Embodiment 3052. A perturbagen for use in the method of any one of Embodiments 3001 to 3051.
Embodiment 3053. A pharmaceutical composition comprising the perturbagen of Embodiment 3052.
Embodiment 3054. A method for inhibiting the formation of a neutrophil, a monocyte, or an immediate progenitor thereof, comprising: exposing a starting population of stem/progenitor cells comprising a non-lineage committed CD34+ cell to a perturbagen that prevents progression of a progenitor cell into and/or or reduces the likelihood that a progenitor cell will progress into a granulocyte monocyte progenitor, neutrophil progenitor, monocyte progenitor or other lineage associated progenitor thereof.
Embodiment 3055. A method of reduction the quantity of neutrophils, monocytes, or immediate progenitors thereof in a population of cells, comprising: exposing a starting population of stem/progenitor cells comprising a non-lineage committed CD34+ cell to a pharmaceutical composition that prevents progression of a progenitor cell into and/or reduces the likelihood that a progenitor cell will progress into a lineage specific progenitor population selected from granulocyte monocyte progenitor, neutrophil progenitor, monocyte progenitor, or other lineage-associated progenitors, the pharmaceutical composition inhibiting the transition of a primitive stem/progenitor population into the lineage specific progenitor population that naturally would have the capacity to differentiate into neutrophils, monocytes or immediate progenitors thereof, wherein the pharmaceutical composition comprises at least one perturbagen selected from Table 6, or a variant thereof.
Embodiment 3056. A method for treating a disease or disorder characterized by an abnormal ratio of neutrophils to monocytes, comprising: (a) administering to a patient in need thereof a therapeutically effective amount of at least one perturbagen selected from Table 6, or a variant thereof, or (b) administering to a patient in need thereof a cell, the cell having been contacted with at least one perturbagen selected from Table 6, or a variant thereof.
Embodiment 3057. The method of Embodiment 3056, wherein the abnormal ratio comprises an increased number of neutrophils and/or an increased number of monocytes.
Embodiment 3058. The method of Embodiment 3056, wherein the abnormal ratio comprises an increased number of neutrophils.
Embodiment 3059. The method of Embodiment 3056, wherein the abnormal ratio comprises an increased number of monocytes.
Embodiment 3060. The method of any one of Embodiments 3056 to 3059, wherein the administering is directed to the bone marrow of the patient.
Embodiment 3061. The method of Embodiment 3061, wherein the administering is via intraosseous injection or intraosseous infusion.
Embodiment 3062. The method of any one of Embodiments 3056 to 3059, wherein the administering the cell is via intravenous injection or intravenous infusion.
Embodiment 3063. The method of any one of Embodiments 3056 to 3062, wherein the administering is simultaneously or sequentially to one or more mobilization agents.
Embodiment 3064. The method of any one of Embodiments 3056 to 3063, wherein the disease or disorder characterized by an abnormal ratio of neutrophils to monocytes is selected from leukocytosis, granulocytosis, monocytosis, and neutrophilia, leukemia (e.g., Chronic myeloid leukemia (CML), Chronic myelomonocytic leukemia (CMML), and Acute myeloblastic leukemia (AML), including AML-M5), cancer induced leukocytosis, drug induced leukocytosis, infection induce leukocytosis, lupus (and other autoimmune disorders showing leukocytosis), and myeloproliferative neoplasms (including polycythemia vera and myelofibrosis).
Embodiment 3065. The method of any one of Embodiments 3056 to 3064, wherein the disease or disorder characterized by an abnormal ratio of neutrophils to monocytes is related to an autoimmune disease, a bone-marrow transplant, a cancer, a genetic disease, a bacterial infection, a viral infection, inflammatory bowel disease, tissue necrosis (e.g., myocardial infarction and burns), acute or chronic inflammation, a previous or concurrent anti-cancer treatment, and/or, a previous or concurrent immune suppressive treatment.
Embodiment 3066. A method for treating a disease or disorder characterized by an abnormal ratio of neutrophils and/or monocytes to progenitor cells, comprising: (a) administering to a patient in need thereof at least one perturbagen selected from Table 6, or a variant thereof, or (b) administering to a patient in need thereof a cell, the cell having been contacted with at least one perturbagen selected from Table 6, or a variant thereof.
Embodiment 3067. The method of Embodiment 3066, wherein the abnormal ratio comprises an increased number of neutrophils and/or an increased number of monocytes and/or a decreased number of progenitor cells.
Embodiment 3068. The method of Embodiment 3067, wherein the abnormal ratio comprises an increased number of neutrophils and an increased number of monocytes.
Embodiment 3069. The method of Embodiment 3067, wherein the abnormal ratio comprises an increased number of neutrophils.
Embodiment 3070. The method of Embodiment 3067, wherein the abnormal ratio comprises a decreased number of progenitor cells.
Embodiment 3071. The method of any one of Embodiments 3066 to 3070, wherein the administering is directed to the bone marrow of the patient.
Embodiment 3072. The method of Embodiment 3071, wherein the administering is via intraosseous injection or intraosseous infusion.
Embodiment 3073. The method of any one of Embodiments 3066 to 3070, wherein the administering the cell is via intravenous injection or intravenous infusion.
Embodiment 3074. The method of any one of Embodiments 3066 to 3073, wherein the administering is simultaneously or sequentially to one or more mobilization agents.
Embodiment 3075. The method of any one of Embodiments 3066 to 3074, wherein the disease or disorder characterized by an abnormal ratio of neutrophils and/or monocytes to progenitor cells is selected from leukocytosis, granulocytosis, monocytosis, and neutrophilia, leukemia (e.g., Chronic myeloid leukemia (CML), Chronic myelomonocytic leukemia (CMML), and Acute myeloblastic leukemia (AML), including AML-M5), cancer induced leukocytosis, drug induced leukocytosis, infection induce leukocytosis, lupus (and other autoimmune disorders showing leukocytosis), and myeloproliferative neoplasms (including polycythemia vera and myelofibrosis).
Embodiment 3076. The method of any one of Embodiments 3066 to 3075, wherein the disease or disorder characterized by an abnormal ratio of neutrophils and/or monocytes to progenitor cells is related to an autoimmune disease, a bone-marrow transplant, a cancer, a genetic disease, a bacterial infection, a viral infection, inflammatory bowel disease, tissue necrosis (e.g., myocardial infarction and burns), acute or chronic inflammation, a previous or concurrent anti-cancer treatment, and/or, a previous or concurrent immune suppressive treatment.
Embodiment 3077. A method for treating monocytosis, comprising: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.
Embodiment 3078. A method for treating neutrophilia, comprising: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.
Embodiment 3079. A method for treating a myeloproliferative neoplasm, comprising: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.
Embodiment 3080. The method of Embodiment 3079, wherein the myeloproliferative neoplasm is polycythemia vera.
Embodiment 3081. The method of Embodiment 3079, wherein the myeloproliferative neoplasm is myelofibrosis.
Embodiment 3082. A method for treating granulocytosis, comprising: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.
Embodiment 3083. The method of Embodiment 3082, wherein the granulocytosis is monocytosis.
Embodiment 3084. The method of Embodiment 3082, wherein the granulocytosis is neutrophilia.
Embodiment 3085. The method of any one of Embodiments 3077 to 3082, wherein the therapeutically effective amount of the at least one perturbagen provides a decrease in the number of neutrophils and/or the number of monocytes in a sample from the human subject relative to the number of neutrophils and/or the number of monocytes in a sample from the human subject prior to administering the at least one perturbagen.
Embodiment 3086. The method of any one of Embodiments 3077 to 3085, wherein the therapeutically effective amount of the at least one perturbagen provides a decrease in the ratio of the number of neutrophils to the number of monocytes in a sample from the human subject relative to the ratio in a sample from the human subject prior to administering the at least one perturbagen.
Embodiment 3087. The method of any one of Embodiments 3077 to 3086, wherein the therapeutically effective amount of the at least one perturbagen provides a decrease in the ratio of the number of monocytes to the number of neutrophils in a sample from the human subject relative to the ratio in a sample from the human subject prior to administering the at least one perturbagen.
Embodiment 3088. The method of any one of Embodiments 3077 to 3087, wherein the therapeutically effective amount of the at least one perturbagen provides an increase in the ratio of the number of neutrophils and/or the number of monocytes to the number of progenitor cells in a sample from the human subject relative to the ratio in a sample from the human subject prior to administering the at least one perturbagen.
Embodiment 3089. The method of any one of Embodiments 3077 to 3088, wherein the administering the therapeutically effective amount of the at least one perturbagen is directed to the bone marrow of the patient.
Embodiment 3090. The method of Embodiment 3089, wherein the administering is via intraosseous injection or intraosseous infusion.
Embodiment 3091. The method of any one of Embodiments 3077 to 3088, wherein the administering the cell is via intravenous injection or intravenous infusion.
Embodiment 3092. The method of any one of Embodiments 3077 to 3091, wherein the administering is simultaneously or sequentially to one or more mobilization agents.
Embodiment 3093. The method of any one of Embodiments 3056 to 3091, wherein the administering occurs about once per day for one or more days.
Embodiment 3094. The method of any one of Embodiments 3056 to 3091, wherein the administering occurs more than once per day for one or more days.
Embodiment 3095. The method of any one of Embodiments 3056 to 3091, wherein the administering occurs at most once per day for one or more days.
Embodiment 3096. The method of any one of Embodiments 3056 to 3091, wherein the administering occurs substantially continuously per administration period.
Embodiment 3097. The method of any one of Embodiments 3056 to 3096, wherein the patient was selected by steps comprising: obtaining from the patient having the disease or disorder a sample of cells comprising a non-lineage committed CD34+ cell; and contacting the sample of cells with least one perturbagen selected from Table 6, or a variant thereof, wherein the at least one perturbagen increases the number of monocytes and/or neutrophils or a progenitor thereof in a culture from the sample of cells.
Embodiment 3098. A method for selecting the patient of any one of Embodiments 3056 to 3096 comprising: obtaining from a subject having the disease or disorder a sample of cells comprising a non-lineage committed CD34+ cell; and contacting the sample of cells with least one perturbagen selected from Table 6, or a variant thereof, wherein when the at least one perturbagen increases the number of monocytes and/or neutrophils or a progenitor thereof in a culture from the sample of cells, the subject is selected as a patient.
Embodiment 3099. Use of the perturbagen of Table 6, or a variant thereof in the manufacture of a medicament for treating a disease or disorder characterized by an abnormal ratio of neutrophils to monocytes. Embodiment 3100. Use of the perturbagen of Table 6, or a variant thereof in the manufacture of a medicament for treating a disease or disorder characterized by an abnormal ratio of neutrophils and/or monocytes to progenitor cells.
Embodiment 3101. A method of identifying a perturbagen, the method comprising: exposing a starting population of progenitor cells, which naturally would have the capacity to undergo a change in cell state comprising differentiating into neutrophils, monocytes and/or immediate progenitors thereof, to a candidate perturbagen; determining an inhibition in the natural capacity to differentiate into neutrophils, monocytes and/or immediate progenitors thereof following exposure of the population of cells to the perturbagen, wherein the candidate perturbagen capable of inhibiting the natural capacity of a progenitor cell to differentiate into a neutrophil, monocyte and/or immediate progenitor thereof is identified as a perturbagen.
Embodiment 3102. A method for making a therapeutic agent for a disease or disorder selected from leukocytosis, granulocytosis, monocytosis, and neutrophilia, leukemia, cancer induced leukocytosis, drug induced leukocytosis, infection induce leukocytosis, lupus (and other autoimmune disorders showing leukocytosis), and myeloproliferative neoplasms (including polycythemia vera and myelofibrosis), comprising: (a) identifying a perturbagen capable of acting as a therapeutic agent for therapy according to the method of Embodiment 3101 and (b) formulating the therapeutic agent for the treatment of the disease or disorder.
   Methods of Culturing Cells in vitro to Perform Single-Cell Analyses

In carrying out the techniques described herein for identifying the causes of cell fate, it is useful to generate datasets regarding cellular-component measurements obtained from single-cells. To generate these datasets, a population of cells of interest may be cultured in vitro. Alternately, these datasets may be generated, from single cells that have not been previously cultured; for example, cells used in single cell analyses may be obtained from dissociated primary tissue or from a blood product. This latter method of generating datasets is often desirable if one wants to capture information of the primary cell/organ as close to the in vivo setting as possible. However, for cells undergoing culturing, single-cell measurements of one or more cellular-components of interest may be performed at one or more time periods during the culturing to generate datasets.

In some embodiments, cellular-components of interest include nucleic acids, including DNA, modified (e.g., methylated) DNA, RNA, including coding (e.g., mRNAs) or non-coding RNA (e.g., sncRNAs), proteins, including post-transcriptionally modified protein (e.g., phosphorylated, glycosylated, myristilated, etc. proteins), lipids, carbohydrates, nucleotides (e.g., adenosine triphosphate (ATP), adenosine diphosphate (ADP) and adenosine monophosphate (AMP)) including cyclic nucleotides such as cyclic adenosine monophosphate (cAMP) and cyclic guanosine monophosphate (cGMP), other small molecule cellular-components such as oxidized and reduced forms of nicotinamide adenine dinucleotide (NADP/NADPH), and any combinations thereof. In some embodiments, the cellular-component measurements comprise gene expression measurements, such as RNA levels.

Any one of a number of single-cell cellular-component expression measurement techniques may be used to collect the datasets. Examples include, but are not limited to single-cell ribonucleic acid (RNA) sequencing (scRNA-seq), scTag-seq, single-cell assay for transposase-accessible chromatin using sequencing (scATAC-seq), CyTOF/SCOP, E-MS/Abseq, miRNA-seq, CITE-seq, and so on. The cellular-component expression measurement can be selected based on the desired cellular-component to be measured. For instance, scRNA-seq, scTag-seq, and miRNA-seq measure RNA expression. Specifically, scRNA-seq measures expression of RNA transcripts, scTag-seq allows detection of rare mRNA species, and miRNA-seq measures expression of micro-RNAs. CyTOF/SCOP and E-MS/Abseq measure protein expression in the cell. CITE-seq simultaneously measures both gene expression and protein expression in the cell. And scATAC-seq measures chromatin conformation in the cell. Table 7 below provides links to example protocols for performing each of the single-cell cellular-component expression measurement techniques described herein.

TABLE 7
Example Measurement Protocols

| Technique | Protocol |
|---|---|
| RNA-seq | Olsen and Baryawno “Introduction to Single-Cell RNA |
| | Sequencing” Current Protocols in Molecular Biology. |
| | Volume122, Issue1, April 2018, e57 |
| Tag-seq | Rozenberg et al., “Digital gene expression analysis with |
| | sample multiplexing and PCR duplicate detection: A |
| | straightforward protocol”, BioTechniques, vol. 61, No. 1, |
| | March 2018 |
| ATAC-seq | Buenrostro et al., “ATAC-seq: A Method for Assaying |
| | Chromatin Accessibility Genome-Wide”, Curr Protoc |
| | Mol Biol. 2015; 109: 21.29.1-21.29.9 |
| miRNA-seq | Faridani et al., “Single-cell sequencing of the small-RNA |
| | transcriptome” Nature Biotechnology volume 34, |
| | pages1264-1266 (2016) |
| CyTOF/ | Bandura et al., “Mass Cytometry: Technique for Real |
| SCoPE-MS/ | Time Single Cell Multitarget Immunoassay Based on |
| Abseq | Inductively Coupled Plasma Time-of-Flight Mass |
| | Spectrometry”, Anal Chem. 2009 Aug 15; 81(16): 6813- |
| | 22Shahi et al., “Abseq: Ultrahigh-throughput single cell |
| | protein profiling with droplet microfluidic barcoding”, |
| | Scientific Reports volume 7, Article number: 44447 |
| | (2017) Budnik et al., “SCOPE-MS: mass spectrometry of |
| | single mammalian cells quantifies proteome heterogeneity |
| | during cell differentiation”, Genome Biology 2018 19: 161 |
| CITE-seq | Stoeckius et al., “Simultaneous epitope and transcriptome |
| | measurement in single cells”, Nature Methods, vol 14, |
| | pages 865-868 (2017) |

The cellular-component expression measurement technique used may result in cell death. Alternatively, cellular-components may be measured by extracting out of the live cell, for example by extracting cell cytoplasm without killing the cell. Techniques of this variety allow the same cell to be measured at multiple different points in time.

If the cell population is heterogeneous such that multiple different cell types that originate from a same “progenitor” cell are present in the population, then single-cell cellular-component expression measurements can be performed at a single time point or at relatively few time points as the cells grow in culture. As a result of the heterogeneity of the cell population, the collected datasets will represent cells of various types along a trajectory of transition.

If the cell population is substantially homogeneous such that only a single or relatively few cell types, mostly the “progenitor” cell of interest, are present in the population, then single-cell cellular-component expression measurements can be performed multiple times over a period of time as the cells transition.

A separate single-cell cellular-component expression dataset is generated for each cell, and where applicable at each of the time periods. The collection of single-cell cellular-component expression measurements from a population of cells at multiple different points in time can collectively be interpreted as a “pseudo-time” representation of cell expression over time for the cell types originating from the same “progenitor” cell. The term pseudo-time is used in two respects, first, in that cell state transition is not necessarily the same from cell to cell, and thus the population of cell provides a distribution of what transition processes a cell of that “progenitor” type is likely to go through over time, and second, that the cellular-component expression measurements of those multiple cell's expressions at multiple time points simulates the possible transition behavior over time, even if cellular-component expression measurements of distinct cells give rise to the datasets. As a deliberately simple example, even if cell X gave a dataset for time point A and cell Y gave a dataset for time point B, together these two datasets represent the pseudo-time of transition between time point A and time point B.

For convenience of description, two such datasets captured for a “same” cell at two different time periods (assuming a technique is used that does not kill the cell) are herein referred to as different “cells” (and corresponding different datasets) because in practice such cells will often be slightly or significantly transitioned from each other, in some cases having an entirely distinct cell type as determined from the relative quantities of various cellular-components. Viewed from this context, these two measurements of a single-cell at different time points can be interpreted as different cells for the purpose of analysis because the cell itself has changed.

Note that the separation of datasets by cell/time period described herein is for clarity of description, in practice, these datasets may be stored in computer memory and logically operated on as one or more aggregate dataset/s (e.g., by cell for all time periods, for all cells and time periods at once).

In some instances, it is useful to collect datasets where a “progenitor” cell of interest has been perturbed from its base line state. There are a number of possible reasons to do this, for example, to knock out one or more cellular-components, to evaluate the difference between healthy and diseased cell states. In these instances, a process may also include steps for introducing the desired modifications to the cells. For example, one or more perturbations may be introduced to the cells, tailored viruses designed to knock out one or more cellular-components may be introduced, CRISPR may be used to edit cellular-components, and so on. Examples of techniques that could be used include, but are not limited to, RNA interference (RNAi), Transcription activator-like effector nuclease (TALEN) or Zinc Finger Nuclease (ZFN).

Depending upon how the perturbation is applied, not all cells will be perturbed in the same way. For example, if a virus is introduced to knockout a particular gene, that virus may not affect all cells in the population. More generally, this property can be used advantageously to evaluate the effect of many different perturbations with respect to a single population. For example, a large number of tailored viruses may be introduced, each of which performs a different perturbation such as causing a different gene to be knocked out. The viruses will variously infect some subset of the various cells, knocking out the gene of interest. Single-cell sequencing or another technique can then be used to identify which viruses affected which cells. The resulting differing single-cell sequencing datasets can then be evaluated to identify the effect of gene knockout on gene expression in accordance with the methods described elsewhere in this description.

Other types of multi-perturbation cell modifications can be performed similarly, such as the introduction of multiple different perturbations, barcoding CRISPR, etc. Further, more than one type perturbation may be introduced into a population of cells to be analyzed. For example, cells may be affected differently (e.g., different viruses introduced), and different perturbations may be introduced into different sub-populations of cells.

Additionally, different subsets of the population of cells may be perturbed in different ways beyond simply mixing many perturbations and post-hoc evaluating which cells were affected by which perturbations. For example, if the population of cells is physically divided into different wells of a multi-well plate, then different perturbations may be applied to each well. Other ways of accomplishing different perturbations for different cells are also possible.

Below, methods are exemplified using single-cell gene expression measurements. It is to be understood that this is by way of illustration and not limitation, as the present disclosure encompasses analogous methods using measurements of other cellular-components obtained from single-cells. It is to be further understood that the present disclosure encompasses methods using measurements obtained directly from experimental work carried out by an individual or organization practicing the methods described in this disclosure, as well as methods using measurements obtained indirectly, e.g., from reports of results of experimental work carried out by others and made available through any means or mechanism, including data reported in third-party publications, databases, assays carried out by contractors, or other sources of suitable input data useful for practicing the disclosed methods.

As discussed herein, gene expression in a cell can be measured by sequencing the cell and then counting the quantity of each gene transcript identified during the sequencing. In some embodiments, the gene transcripts sequenced and quantified may comprise RNA, for example mRNA. In alternative embodiments, the gene transcripts sequenced and quantified may comprise a downstream product of mRNA, for example a protein such as a transcription factor. In general, as used herein, the term “gene transcript” may be used to denote any downstream product of gene transcription or translation, including post-translational modification, and “gene expression” may be used to refer generally to any measure of gene transcripts.

Although the remainder of this description focuses on the analysis of gene transcripts and gene expression, all of the techniques described herein are equally applicable to any technique that obtains data on a single-cell basis regarding those cells. Examples include single-cell proteomics (protein expression), chromatin conformation (chromatin status), methylation, or other quantifiable epigenetic effects.

The following description provides an example general description for culturing a population of cells in vitro in order to carry out single-cell cellular-component expression measurement multiple time periods. Methods for culturing cells in vitro are known in the art. Those of skill in the art will also appreciate how this process could be modified for longer/shorter periods, for additional/fewer single-cell measurement steps, and so on.

In one embodiment, the process for culturing cells in a first cell state into cells in a second cell state includes one or more of the following steps:
Day 0: Thaw cells in the first cell state into a plate in a media suitable for growth of the cells.
Day 1: Seed cells in the first cell state into a multi-well plate. If applicable, perform additional steps to affect gene expression by cells. For example, simultaneously infect with one or more viruses to activate or knock out genes of interest.

Perform gene expression measurement iteration t₁ for cells in the wells.
Day 1+1: Change media as needed if any additional processes are performed.

If applicable, perform gene expression measurement iteration tᵢ for cells in the wells.
Day 1+m: Change media to media appropriate to support growth of cells in the second cell state. If applicable, perform gene expression measurement iteration tₘ for cells in the wells.

Days 1+n, o, p, etc.: Media change as needed to support further cell state transition from the first cell state to the second cell state. If applicable, perform additional steps to affect further transition from the first cell state to the second cell state. For example, add perturbations of interest to push cells towards the second cell state.
If applicable, perform gene expression measurement iterations tₙ, tₒ, tₚ, etc., for cells in the wells.

Day q: Perform gene expression measurement iteration t_{q} for cells in the wells and in the second state.
Collect cells into a tube and stain in suspension with antibodies matched to genes/proteins of interest, thereby sorting/identifying cells without having to lyse/destroy them. This step also can identify surface proteins that might not be seen with as much resolution in the setting of the cytoplasm. Image with a cell imaging system such as the BD Celestra flow cytometer or similar instrument by acquiring the cells from each well or tube. Quantify of number of cells per well that are in the first cell state and the number of cells per well that are in the second cell state. These steps can be used with unfixed cells.

Any aspect or embodiment disclosed herein can be combined with any other aspect or embodiment as disclosed herein.

### Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Any terms not directly defined herein shall be understood to have the meanings commonly associated with them as understood within the art of the disclosure. Certain terms are discussed herein to provide additional guidance to the practitioner in describing the compositions, the devices, the methods and the like of aspects of the disclosure and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

The term “perturbation” in reference to a cell (e.g., a perturbation of a cell or a cellular perturbation) refers to any treatment of the cell with one or more active agents capable of causing a change in the cell's lineage or cell state (or in the lineage or cell state of the cell's progeny). In preferred embodiments, a perturbagen promotes progression of a progenitor cell into and/or or increases the likelihood that a progenitor cell will progress into a monocyte or a neutrophil lineage, e.g., into a lineage specific progenitor cell selected from granulocyte monocyte progenitor, neutrophil progenitor, monocyte progenitor, or another lineage-associated progenitor. In embodiments, a perturbagen prevents progression of a progenitor cell into and/or or reduces the likelihood that a progenitor cell will progress into a monocyte or a neutrophil lineage, e.g., into a lineage specific progenitor cell selected from granulocyte monocyte progenitor, neutrophil progenitor, monocyte progenitor, or another lineage-associated progenitor. These active agents can be referred to as “perturbagens.” In embodiments, the perturbagen can comprise, e.g., a small molecule, a biologic, a protein, a protein combined with a small molecule, an antibody-drug conjugate (ADC), a nucleic acid, such as an siRNA or interfering RNA, a cDNA over-expressing wild-type and/or mutant shRNA, a cDNA over-expressing wild-type and/or mutant guide RNA (e.g., Cas9 system or other gene editing system), or any combination of any of the foregoing. As used herein, a perturbagen classified as a “compound” may be a small molecule or a biologic. Also, a perturbagen classified as “overexpression of gene” may be cDNA over-expressing a wild-type gene or an mRNA encoding a wild-type gene. In embodiments, an mRNA may comprise a modified nucleotide that promotes stability of the mRNA and/or reduces toxicity to a subject. Examples of modified nucleotides useful in the present disclosure include pseudouridine and 5-methylcytidine. Where a perturbagen is (or includes) a nucleic acid or protein described by reference to a particular sequence, it should be understood that variants with similar function and nucleic acid or amino acid identity are encompassed as well, e.g., variants with about 1%, 2%, 3%, 4%, 5%, 6%, 7%, 8%, 9%, 10%, 11%, 12%, 13%, 14%, 15%, or more, variation, i.e., having about 99%, 98%, 97%, 96%, 95%, 94%, 93%, 92%, 91%, 90%, 89%, 88%, 87%, 86%, or 85% identity to the reference sequence; e.g., in some embodiments, having, for example, at least: 1, 2, 3, 4, 5, 6, 7, 8, 9, 10, 11, 12, 13, 14, 15, 20, 25, 30, 35, 40, 45, 50, or more, substitutions.

The term “progenitor” in reference to a cell (e.g., a progenitor cell) refers to any cell that is capable of transitioning from one cell state to at least one other cell state. Thus, a progenitor can differentiate into one or more cell types and/or can expand into one or more types of cell populations.

As used herein, the terms “cell fate” and “cell state” are interchangeable and synonymous.

The term “subject,” refers to an individual organism such as a human or an animal. In embodiments, the subject is a mammal (e.g., a human, a non-human primate, or a non-human mammal), a vertebrate, a laboratory animal, a domesticated animal, an agricultural animal, or a companion animal. In embodiments, the subject is a human (e.g., a human patient). In embodiments, the subject is a rodent, a mouse, a rat, a hamster, a rabbit, a dog, a cat, a cow, a goat, a sheep, or a pig.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”. Likewise, the term “and/or” covers both “or” and “and”.

Unless specifically stated or obvious from context, as used herein, the term “about” is understood as within a range of normal tolerance in the art, for example within 2 standard deviations of the mean. About is understood to be within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The disclosure will be further described in the following examples, which do not limit the scope of the disclosure described in the claims.

### EXAMPLES

### Example 1: Single Cell Gene Expression Profiling of In Vitro Hematopoietic Differentiation

To develop a model of early hematopoietic differentiation, mobilized peripheral blood (mPB) CD34+ cells were thawed, cultured and subjected to single cell gene expression profiling (GEP) at three (3) timepoints throughout the differentiation process. Briefly, three (3) cryopreserved vials of mPB CD34+donors were thawed. Approximately 1×10⁵ cells from each donor were aliquoted for single cell and bulk RNA sequencing, as described below. Donors 1 and 2 and Donors 2 and 3 were pooled and cultured for five days in expansion media comprising StemSpan Serum Free media (Stem Cell Technologies Cat #09650) and CC100 cocktail (Stem Cell Technologies Cat #02690), which contains SCF, FIt3L, IL-3, and IL-6, which promotes expansion and non-directed lineage differentiation. Two parallel differentiation runs were performed: donor 1+2 and donor 2+3, which allowed, following donor deconvolution post-sequencing, detection of differentiation run or donor specific differences throughout the differentiation process. At three time points: E0 (freshly thawed), E3 (Expansion phase day 3) and E5, cells were collected for counts and single cell and bulk RNA sequencing.

Bulk mRNA sequencing was performed separately on three donors at E0 (time of thaw). This was used to deconvolute the single cell samples using donor specific Single-nucleotide polymorphisms (SNPs). Deconvolution of donor contribution using donor specific SNPs was performed as part of the downstream analysis.

For the subsequent time points, the two pooled donor differentiation runs were sequenced independently for bulk RNA sequencing (two sequencing runs: 1+2, 2+3) and both differentiation runs were pooled for single cell mRNA sequencing (one pool: 1+2+2+3).

RNA isolation was performed for bulk total RNA sequencing using the miRNeasy Micro Kit (50) QIAGEN 217084 kit. RNA was quantified using the Qbit and Bioanalyzer.

Bulk total RNAseq NGS libraries were prepared using the KAPA Stranded RNA-Seq with RiboErase kit (Kapa BioSystems).

Paired end (2×125) total bulk RNA sequencing (mRNA, isoforms, SNPs, non-coding RNA) was performed on the Illumina HiSeq 2500 using the High Output v4 single lane flow cells.

For single cell sequencing at each time point, all donors were pooled into a single tube prior to running through the 10× Genomics Single Cell 3′ v2 workflow steps. Cells were washed, counted, and run within one hour using 10× partitioning and cDNA amplification (10× Genomics, Single Cell 3′ v2). The rest of the library prep was completed as per the 10× Genomics Single Cell 3′ v2 library prep protocol. 10× samples were sequenced using three NextSeq High 1×75 flow cell runs.

Count matrices were obtained by running Cell Ranger (10× Genomics) with default settings. Low quality cells with less than 200 genes expressed were filtered, genes expressed in less than three cells were filtered, and data was CPM normalized and log-transformed. Highly variable genes were detected, and the count matrices were corrected for mitochondrial gene expression. A dimensionality-reduced representation of the count matrix was embedded using UMAP, clustered using Louvain clustering and annotated using differential expression testing (Mann Whitney U test) and comparisons to established marker genes. Proxies for cellular states are the annotated clusters, as shown in FIG. 1A. Differential signatures for the 10 to 9 transitions, i.e., from a non-lineage committed CD34+ progenitor cell to cells of the granulocyte monocyte progenitor (GMP) lineage, were then used to predict perturbations that would promote or inhibit the transition.

### Example 2: Modeling Neutrophil/Monocyte Differentiation

The single cell gene expression profiling (GEP) of Example 1 was used to develop a model of in vitro neutrophil/monocyte differentiation through a proposed granulocyte monocyte progenitor (GMP, Cluster 9) (see, FIG. 1A). Differentiation proceeded based on cell-by-cell transcriptional similarity in a sequential fashion from the beginning to the end of the differentiation process. During the expansion phase, progenitors of the myeloid lineages including: granulocyte, neutrophil, mast/basophil, erythrocyte, and megakaryocyte progenitors were all observed, along with lineage specific transcriptional signatures (FIG. 1A).

### Example 3A: Identification of Candidate Perturbations that Promote GMP Differentiation

Using the model described in Example 2, it was hypothesized that differentiation towards the GMP cluster (cluster 9 of FIG. 1A) could be promoted by increasing expression of genes specific to cells of the GMP cluster; in other words, the goal was to replicate the gene signatures found in cells of cluster 9 during a progenitor cell's first five days in culture. To do this, the differential transcription factor gene expression between clusters 9 (GMP) and 10 (non-lineage committed CD34+ population at E3) was determined and evaluated against a library of cellular gene signatures. Genes found to be positively associated with promoting GMP lineage and/or GMP differentiation are listed in Table 1 and genes found to be positively associated with blocking GMP lineage and/or GMP differentiation are listed in Table 2.

From this, experiments were conducted by providing agents that altered expression of the genes listed in Table 1 or Table 2. This resulted in a list of perturbagens (including agents that regulate gene targets, ligands, and small molecules) that were predicted to promote the GMP lineage and/or GMP differentiation through activation or inhibition of lineage specific differentiation programs.

The list of perturbagens that were predicted to be associated with promoting GMP lineage and/or GMP differentiation is provided in Table 3.

### Example 3B: Identification of Candidate Perturbations that Inhibit GMP Differentiation

Using the model described in Example 2, it was hypothesized that differentiation towards the GMP cluster (cluster 9 of FIG. 1A) could be inhibited by increasing expression of genes specific to cells of the non-lineage committed CD34+ progenitor cell rather than increasing expression of genes specific to the GMP cluster. In other words, the goal was to identify the gene signatures found in cells of cluster 9 during a progenitor cell's first five days in culture in which the gene signature includes genes having increased expression (which likely promote the transition towards the GMP cluster) and genes having decreased expression (which likely inhibit the transition towards the GMP cluster); of interest in this example were genes showing reduced expression as a progenitor cell progresses into a cell of the GMP cluster. To do this, the differential transcription factor gene expression between clusters 9 (GMP) and 10 (non-lineage committed CD34+ population at E3) was determined and evaluated against a library of cellular gene signatures. Genes found to be positively associated with inhibiting GMP lineage progression and/or GMP differentiation are listed in Table 4 and genes found to be positively associated with promoting GMP lineage progression and/or GMP differentiation are listed in Table 5.

From this, experiments were conducted by providing agents that altered expression of the genes listed in

Table 4 or Table 5. This resulted in a list of perturbagens that were predicted to prevent progression of a progenitor cell into and/or reduce the likelihood that a progenitor cell will progress into the GMP lineage and/or GMP differentiation through activation or inhibition of lineage specific differentiation programs.

A list of perturbagens that were predicted to be associated with inhibiting GMP lineage and/or GMP differentiation is provided in Table 6.

### Example 4A: Testing Perturbagens in In Vitro Culture

Steps used in this example are illustrated in FIG. 1B. Briefly, mobilized peripheral blood (mPB) derived CD34+ hematopoietic stem and progenitor cells (HSPCs) were thawed and allowed to recover in StemSpan SFEM (Stem Cell Technologies) supplemented with TPO and CC100 cytokine supplement (Stem Cell Technologies) that contains SCF, FIt3L, IL-6 and IL-3 (Expansion media) for 48 hours prior to delivery of the perturbagens. Perturbagens (including those listed in Table 3) reconstituted in DMSO were added to media at different concentrations. Cells were cultured for 48 hours in the presence of the perturbagens prior to collection for cell counts, viability, and flow cytometric analyses to assess the impact the perturbagens on the cell population's composition. In the illustration of FIG. 1B, results from treatments with perturbagens relevant to the present disclosure and which drive non-lineage committed CD34+ cells towards the GMP lineages are shown in the bottom left scatter plot; results from control perturbagens that drive the non-lineage committed CD34+ cells away from the GMP lineages are shown in the bottom right scatter plot. These steps could likewise be used on cord blood (CB), or bone marrow (BM) derived CD34+ hematopoietic stem and progenitor cells (HSPCs).

Expansion of GMPs was measured by expression of CD66b+ (for neutrophil) and CD64+ (for monocyte) progenitors within the CD34+CD38+/−gate (encompassing hematopoietic stem cells progenitors) relative to vehicle control conditions (DMSO). In addition, culture conditions promoting neutrophil (StemSpan+SCF, TPO, G-CSF and GM-CSF) and monocyte (StemSpan+SCF, TPO, Flt3, M-CSF, GM-CSF) differentiation were included to demonstrate that lineage specific changes could be measured following days in culture.

Changes in the ratios of specific cell types in a population following 48 hour treatments with a published cocktail of cytokines (perturbagens) is shown in FIG. 1C. As shown in the second column from the right, the perturbagens that drove non-lineage committed CD34+ cells towards the GMP lineages and also specifically promoted the monocyte cell state results in an increase in the fraction of monocytes in a population of cells relative to the control treatments (NT control or DMSO); similarly, as shown in the right most column, the perturbagens that drove non-lineage committed CD34+ cells towards the GMP lineages and also specifically promoted the neutrophil cell state results in an increase in the fraction of neutrophils in a population of cells relative to the control treatments. Results from perturbagens that drive the non-lineage committed CD34+ cells away from the GMP lineages (e.g., towards the erythroid lineage or megakaryocyte lineage) are shown, respectively, in the second and third columns of FIG. 1C.

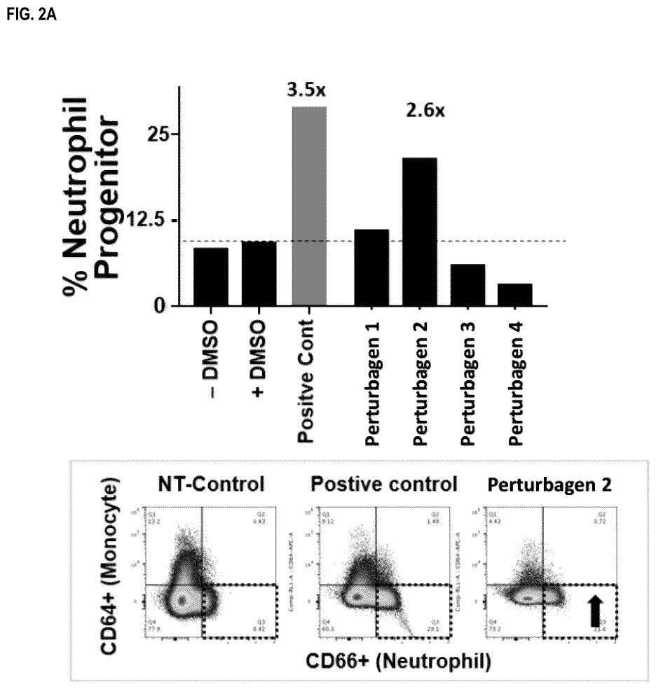

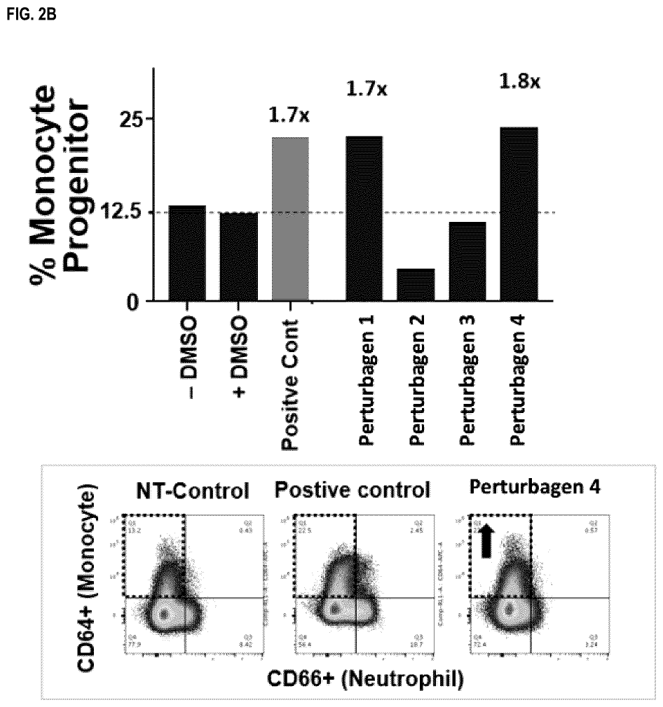

Three of four perturbagens (of Table 3) were shown to promote the GMP lineage and resulted in increased numbers of neutrophils and/or monocyte progenitors 48 hours after addition of the perturbagen (FIG. 2A and FIG. 2B). Surprisingly, perturbagens showing lineage specificity were observed. For example, Perturbagen 1 and Perturbagen 4 promoted the monocyte lineage but not the neutrophil lineage, whereas Perturbagen 2 promoted the neutrophil lineage but not the monocytic lineage. In FIG. 2A or FIG. 2B, the positive controls which promoted neutrophil differentiation (FIG. 2A) included StemSpan+SCF, TPO, G-CSF and GM-CSF and the positive controls which promoted monocyte differentiation (FIG. 2B) included StemSpan+SCF, TPO, Flt3, M-CSF, GM-CSF.

The ability to specifically promote neutrophil and/or monocyte lineages would be valuable in designing a therapeutic composition. More specifically, for a disease characterized by a reduced number of neutrophils, a therapeutic composition comprising a perturbagen that increases the number of neutrophils could be beneficial; on the other hand, a disease that would benefit from increased numbers of monocytes could be treated by a therapeutic composition comprising a perturbagen that increases the number of monocytes.

In this and the above examples, antibodies directed against hematopoietic surface proteins and conjugated fluorophores are listed below in Table 8.

TABLE 8

| Antibody | Fluorophore | Vendor | Clone | Cat # |
|---|---|---|---|---|
| CD41a | FITC | eBiosciences | HIP8 | 11-0419-42 |
| CD66b | Percp5.5 | Biolegend | G10F5 | 305108 |
| CD71 | PE | eBiosciences | OKT9 | 12-0719-42 |
| FcεRlα+ | PE-Cy7 | eBiosciences | AER-37 | 25-5899-42 |
| CD64 | APC | Biolegend | 10.1 | 305014 |
| CD38 | AF700 | eBiosciences | HIT2 | 56-0389-42 |
| CD34 | APC-eFlur780 | eBiosciences | 4H11 | 47-0349-42 |
| CD123 | BV421 | Biolenged | 6H6 | 306018 |
| CD135 | BV421 | BD | 4G8 | BD564708 |
| CD45RA | BV785 | BD | Hi100 | BDV563870 |

In this and the above examples, flow cytometry panel comparisons with either CD135 or CD123 to identify myeloid progenitor populations and/or to identify cell states are listed below in Table 9.

TABLE 9

| | | Immunophenotype (Lin−) |
|---|---|---|
| Cell Type | Immunophenotype (Lineage−) CD135 | CD123 |
| Megakaryocyte-erythroid | CD34 + CD38 + CD45RA − CD135 − CD71+ | CD34 + CD38 + CD45RA − CD123 − |
| progenitor; megakaryocyte | | CD71Low |
| progenitor (MEP) | | |
| Erythroid progenitor | CD34 + CD38 + CD45RA − CD135 − | CD34 + CD38 + CD45RA − CD123 − |
| | CD71 + KEL+ | CD71 + CD41− |
| Megakaryocyte progenitor | CD34 + CD38 + CD45RA − CD135− | CD34 + CD38 + CD45RA − CD123 − |
| | CD71 + KEL− | CD71 + CD41+ |
| Common myeloid progenitor | CD34 + CD38 + CD45RA − CD135+ | CD34 + CD38 + CD45RA − CD123+ |
| (CMP; immature populations - | | |
| myeloid and early stage | | |
| Neutrophil) | | |
| Eosinophil/basophil/mast cell | CD34 + CD38 + CD45RA − CD135mid | CD34 + CD38 + CD45RA − CD123+ |
| progenitor | | |
| Mast cell/basophil | CD34 + CD38 + CD45RA − CD135mid | CD34 + CD38 + CD45RA − |
| | FcεRlα+ | CD123 + FcεRlα+ |
| GMP (neutrophil progenitor; | CD34 + CD38 + CD45RA + CD135+ | CD34 + CD38 + CD45RA + CD123+ |
| monocyte/DC progenitor) | | |
| Neutrophil progenitor | CD34 + CD38 + CD45RA + CD135 + CD66b+ | CD34 + CD38 + CD45RA + CD123 + |
| | | CD66b + CD64− |
| Monocyte progenitor | CD34 + CD38 + CD45RA + CD135 + CD64+ | CD34 + CD38 + CD45RA + CD123 + |
| | | CD66b − CD64+ |

NOTE:
Lineage: Lineage−: CD4 − CD8 − CD11b − CD14 − CD19 − CD20 − CD56 − CD10− (this stain is optional if working with purified CD34+ cells)

### Example 4B: Testing Perturbagens in In Vitro Culture

Steps used in this example are illustrated in FIG. 1B. Briefly, mobilized peripheral blood (mPB) derived CD34+ hematopoietic stem and progenitor cells (HSPCs) were thawed and allowed to recover in StemSpan SFEM (Stem Cell Technologies) supplemented with TPO and CC100 cytokine supplement (Stem Cell Technologies) that contains SCF, FIt3L, IL-6 and IL-3 (Expansion media) for 48 hours prior to delivery of the perturbagens. Perturbagens (including those listed in Table 6) reconstituted in DMSO were added to media at different concentrations. Cells were cultured for 48 hours in the presence of the perturbagens prior to collection for cell counts, viability, and flow cytometric analyses to assess the impact the perturbagens on the cell population's composition. In the illustration of FIG. 1B, results from control perturbagens which drive non-lineage committed CD34+ cells towards the GMP lineages are shown in the bottom left scatter plot; treatments with perturbagens relevant to the present disclosure and that drive the non-lineage committed CD34+ cells away from the GMP lineages are shown in the bottom right scatter plot. These steps could likewise be used on cord blood (CB), or bone marrow (BM) derived CD34+ hematopoietic stem and progenitor cells (HSPCs).

Expansion of GMPs was measured by expression of CD66b+ (for neutrophil) and CD64+ (for monocyte) progenitors within the CD34+CD38+/−gate (encompassing hematopoietic stem cells progenitors) relative to vehicle control conditions (DMSO). In addition, culture conditions promoting neutrophil (StemSpan+SCF, TPO, G-CSF and GM-CSF) or monocyte (StemSpan+SCF, TPO, Flt3, M-CSF, GM-CSF) differentiation were included to demonstrate that lineage specific changes could be measured following days in culture.

Changes in the ratios of specific cell types in a population following 48 hour treatments with a published cocktail of cytokines (control perturbagens) is shown in FIG. 1C. Results from perturbagens useful in the present disclosure and that drive the non-lineage committed CD34+ cells away from the GMP lineages (e,g., towards the erythroid lineage or megakaryocyte lineage) are shown, respectively, in the second and third columns of FIG. 1C.

As shown in the second column from the right, the control perturbagens that drove non-lineage committed CD34+ cells towards the GMP lineages and also specifically promoted the monocyte cell state results in an increase in the fraction of monocytes in a population of cells relative to the NT control or DMSO treatments; similarly, as shown in the right most column, the control perturbagens that drove non-lineage committed CD34+ cells towards the GMP lineages and also specifically promoted the neutrophil cell state results in an increase in the fraction of neutrophils in a population of cells relative to the NT control or DMSO treatments.

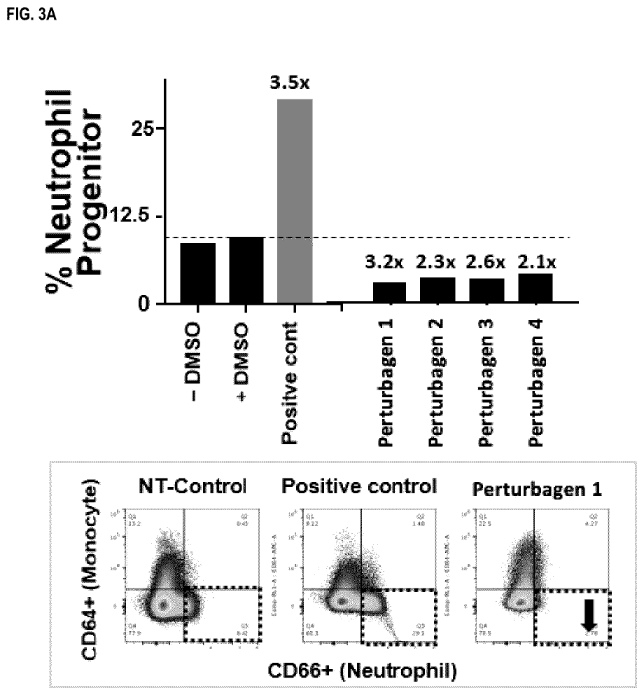

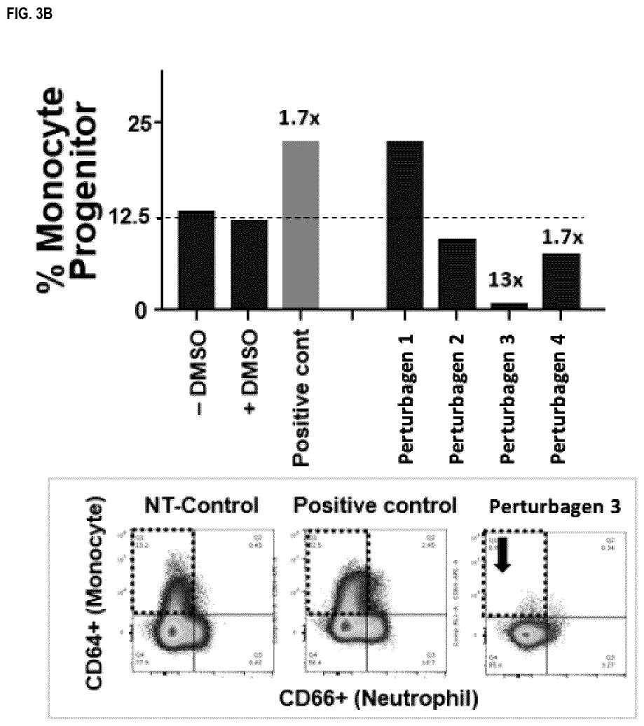

Four perturbagens (of Table 6) were shown to inhibit progression toward the GMP lineage and resulted in decreased numbers of neutrophils and/or monocyte progenitors 48 hours after addition of the perturbagen (FIG. 3A and FIG. 3B). Surprisingly, perturbagens showing lineage specificity were observed. For example, Perturbagen 1, Perturbagen 2, Perturbagen 3, and Perturbagen 4 inhibited the neutrophil lineage, whereas Perturbagen 3 and Perturbagen 4 inhibited both the monocytic lineage and the neutrophil lineage. In FIG. 3A or FIG. 3B, the positive controls which promoted neutrophil differentiation (FIG. 3A) included StemSpan+SCF, TPO, G-CSF and GM-CSF and the positive controls which promoted monocyte differentiation (FIG. 3B) included StemSpan+SCF, TPO, Flt3, M-CSF, GM-CSF.

The ability to specifically inhibit neutrophil and/or monocyte lineages would be valuable in designing a therapeutic composition. More specifically, for a disease characterized by an increased number of neutrophils, a therapeutic composition comprising a perturbagen that decreases the number of neutrophils could be beneficial; on the other hand, a disease that would benefit from decreased numbers of monocytes could be treated by a therapeutic composition comprising a perturbagen that increases the number of monocytes.

In this and the above examples, antibodies directed against hematopoietic surface proteins and conjugated fluorophores are listed above in Table 8.

In this and the above examples, flow cytometry panel comparisons with either CD135 or CD123 to identify myeloid progenitor populations and/or to identify cell states are listed below in Table 9.

### Example 5: Testing Functional Impacts of Perturbagens in In Vitro Colony Forming Culture Assays

Methocult is a semi-solid methylcellulose culture system containing a defined mixture of human cytokines (rh SCF, rh GM-CSF, rh IL-3, rh G-CSF, and rh EPO), which allows for the detection of hematopoietic progenitors (CFU-E, BFU-E, CFU-GM, CFU-G, CFU-M, and CFU-GEMM) in bone marrow (BM), cord blood (CB), peripheral blood (PB), or mobilized peripheral blood (mPB) cells following 14-16 days in culture. The CFU assay provides an in vitro functional readout that complements the phenotypic readout described in Example 2.

Briefly, mPB, CB, or BM derived CD34+ hematopoietic stem and progenitor cells are thawed and allowed to recover in StemSpan SFEM (Stem Cell Technologies) supplemented with TPO and CC100 cytokine supplement (Stem Cell Technologies) that contains SCF, FIt3L, IL-6 and IL-3 (Expansion media) for 48 hours prior to resuspension in MethoCult™ H4034 Optimum (Stem Cell Technologies) and perturbagens at a range of concentrations based on previous liquid culture experiments (in Examples 3A and 3B and as shown in Table 3 or Table 6). Here, changes in number and/or size of the above colony types following exposure of human CD34+ cells to the perturbagens predicted to change the frequency of erythroid (CFU-E, BFU-E) and myeloid colonies (CFU-GM) are tested. Furthermore, these assays enumerate the frequency of myeloid colonies towards or away from the granulocyte (CFU-G) or monocyte/macrophage (CFU-M) lineage providing a functional assay to validate the ability of the predicted perturbagens to direct or inhibit hematopoietic differentiation.

### Example 6A: Testing perturbagens in vivo

Following in vitro validation of perturbagens that promoted promotion of GMPs and differentiation of cells in the GMP lineage, perturbagens are further tested in vivo using C57BL/6 mice. Here, the perturbagens' ability to promote or block neutrophil or monocyte lineage in vivo during steady state hematopoiesis or following sub-lethal myeloablation, induced by busulfan treatment or whole body irradiation (e.g., X-ray irradiation) is assayed. Busulfan is a DNA alkylating reagent that clinically used for bone marrow conditioning yet has reduced toxicity. Effective busulfan dosing has been established in murine models and has been shown to be a less toxic conditioning method and an alternative to whole body irradiation.

Briefly, a single dose or repeat doses (2-3 doses) of one or more perturbagen is injected i.p or i.v into 8-10 week old female C57/BI6 mice. Mice either are undergoing normal steady state hematopoiesis or have been provided busulfan-mediated conditioning (sub-lethal myeloablation). For repeat dosing, perturbagens are injected every two days (48 hours) for three doses. Peripheral blood is collected every 1-2 days post-injection for 2-4 weeks. Flow cytometry analyses are performed to measure the number and ratios of neutrophil and monocyte cells within the peripheral blood. 2-4 weeks later, mice are sacrificed and analyses of the bone marrow and spleen are performed using standard flow cytometry panels to determine the impact of the pertubagens on neutrophils and monocytes numbers and ratios and to determine the impact of the perturbagens on hematopoietic stem cells (HSC) and progenitor populations including multipotent progenitor cells (MPPs) and GMPs.

In this example, antibodies directed against murine hematopoietic surface proteins and conjugated fluorophores are listed below in Table 10.

TABLE 10

| Antibody | Fluorophore | Vendor | Clone |
|---|---|---|---|
| CD3e | biotin | eBiosciences | 145-2C11 |
| CD19 | biotin | eBiosciences | MB19-1 |
| Gr1 | biotin | eBiosciences | RB6-685 |
| CD11b (Mac1) | biotin | eBiosciences | |
| Ter119 | biotin | eBiosciences | TER119 |
| IL7Ra (CD127) | biotin | | A7R34 |
| Ly6B.2 (7/4) | | Miltenyi | |
| Ly6G | | eBiosciences | 1A8 |
| CD3e | | eBiosciences | 145-2C11 |
| CD19 | | eBiosciences | MB19-1 |
| Gr1 | | eBiosciences | RB6-685 |
| CD11b (Mac1) | | eBiosciences | M1/70 |
| Ter119 | | eBiosciences | TER119 |
| CD150 | PE/Cy5 | Biolegend | TC15-12F12.2 |
| CD41 | BV605 | Biolegend | MwReg30 |
| CD117 (Kit) | APC | eBiosciences | 2B8 |
| Ly6a (Sca1) | PE/Cy7 | eBiosciences | D7 |
| CD34 | AlexaFluor-647 | eBiosciences | |
| CD16/32 | AlexaFlour-700 | eBiosciences | |

In this example, murine flow cytometry panel comparisons to identify stem cells, myeloid progenitor, neutrophil, and/or monocyte populations are listed below in Table 11.

TABLE 11

| Cell Type | Immunophenotype |
|---|---|
| Granulocytes* | Ly6G + Ly6B.2 + B220 − Ter119− |
| Monocytes/ | Ly6C^{Lo-neg}Ly6G⁻SSC^{Lo} B220 − Ter119− |
| Macrophages* | and |
| | Ly6C⁺Ly6G⁻SSC^{Lo} B220 − Ter119− |
| Neutrophil* | Ly6G^{Hi}SSC^{Int} B220 − Ter119− |
| GMP | Lin-c-Kit + Sca-1 − CD34 + CD16/32+ |
| | (Lineage−: CD3e, CD19, Gr1, CD11b, Ter119, IL7Ra) |
| MPP1 | Lin − Kit + Sca1 + Fkt3 − CD150 − CD48− |
| | (Lineage−: CD3e, CD19, Gr1, CD11b, Ter119, IL7Ra) |
| LT-HSC | Lin − Kit + Sca1 + Flt3 − CD150 + CD48− (±CD41). |
| | (Lineage−: CD3e, CD19, Gr1, CD11b, Ter119, IL7Ra) |

NOTE:
*Lineage−: Ter119 − CD4, CD8, B220, NK1.1, CD11c^{Hi})

### Example 6B: Testing Perturbagens In Vivo

Following in vitro validation of perturbagens that prevent progression of a progenitor cell into and/or reduce the likelihood that a progenitor cell will progress into the GMP lineage, perturbagens are further tested in vivo using C57BL/6 mice. Here, the perturbagens' ability to block neutrophil or monocyte lineage in vivo during steady state hematopoiesis is assayed.

Briefly, a single dose or repeat doses (2-3 doses) of one or more perturbagen is injected i.p or i.v into 8-10 week old female C57/BI6 mice. Mice either are undergoing normal steady state hematopoiesis or are mice which produce excess neutrophils and/or monocytes due to a genetic mutation, an infection, or a treatment with a suitable agent that induces inflammation. For repeat dosing, perturbagens are injected every two days (48 hours) for three doses. Peripheral blood is collected every 1-2 days post-injection for 2-4 weeks. Flow cytometry analyses are performed to measure the number and ratios of neutrophil and monocyte cells and/or ratios of neutrophil and/or monocyte cells to progenitor cells within the peripheral blood. 2-4 weeks later, mice are sacrificed and analyses of the bone marrow and spleen are performed using standard flow cytometry panels to determine the impact of the pertubagens on neutrophil, monocytes, and/or progenitor cell numbers and ratios and to determine the impact of the perturbagens on hematopoietic stem cells (HSC) and progenitor populations including multipotent progenitor cells (MPPs) and GMPs.

In this example, antibodies directed against murine hematopoietic surface proteins and conjugated fluorophores are listed above in Table 10.

In this example, murine flow cytometry panel comparisons to identify stem cells, myeloid progenitor, neutrophil, and/or monocyte populations are listed above in Table 11.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

### EQUIVALENTS

While the disclosure has been disclosed in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A method for directing a change in cell state of a progenitor cell comprising:
contacting a population of cells comprising a progenitor cell with at least one perturbagen selected from Table 3, or a variant thereof,
wherein the at least one perturbagen is capable of directing a gene signature in the progenitor cell; and
wherein the progenitor cell is a non-lineage committed CD34+ cell.

2. A method for directing a change in cell state of a progenitor cell, comprising:
contacting a population of cells comprising a progenitor cell with at least one perturbagen capable of altering a gene signature in the progenitor cell,
wherein altering the gene signature comprises an increase in expression and/or activity in the progenitor cell of one or more genes selected from Table 1 and/or a decrease in expression and/or activity in the progenitor cell of one or more genes selected from Table 2 and wherein the progenitor cell is a non-lineage committed CD34+ cell.

3. A method for directing a change in cell state of a progenitor cell, comprising:
contacting a population of cells comprising a progenitor cell with at least one perturbagen selected from Table 3, or a variant thereof, and capable of altering a gene signature in the progenitor cell, wherein altering the gene signature comprises an increase in expression and/or activity in the progenitor cell of one or more genes selected from Table 1 and/or a decrease in expression and/or activity in the progenitor cell of one or more genes selected from Table 2 and
wherein the progenitor cell is a non-lineage committed CD34+ cell.

4. The method of any one of claims 1 to 3, wherein the change in cell state provides one or more of the following:
a) an increase in the number of neutrophils and/or the number of monocytes, optionally wherein the increase in the number of neutrophils and/or the number of monocytes is relative to the number of neutrophils and/or the number of monocytes obtained from a population of progenitor cells that is not contacted with the at least one perturbagen or relative to the population of progenitor cells prior to contacting with the at least one perturbagen; and/or
b) the number of progenitor cells is decreased, optionally wherein the decrease in the number of progenitor cells is relative to the number of progenitor cells in a population of progenitor cells that is not contacted with the at least one perturbagen or relative to the number of progenitor cells in the population prior to contacting with the at least one perturbagen; and/or
c) the number of progenitor cells is increased, optionally wherein the increase in the number of progenitor cells is relative to the number of progenitor cells in a population of progenitor cells that is not contacted with the at least one perturbagen or relative to the number of progenitor cells in the population prior to contacting with the at least one perturbagen.

5. The method of any one of claims 1 to 4, wherein the number of CD66b+ neutrophil progenitors, CD64+monocytic progenitors, neutrophilic promyelocytes, neutrophilic myelocytes, neutrophilic metamyelocytes, and/or neutrophilic band cells is increased and/or the number of CD34+CD38+/− cells are increased about two days after contacting the population of cells comprising a CD34+ cell with the at least one perturbagen and/or the number of CD34-CD38+/− cells are increased about five days after contacting the population of cells comprising a CD34+ cell with the at least one perturbagen.

6. The method of any one of claims 1 to 4, wherein the number of monoblasts and/or promonocytes is increased and/or the number of granulocyte-monocyte progenitor (GMP) cells is increased, optionally wherein the progenitor cell for the GMP cell is a common myeloid progenitor (CMP); and/or the number of monocyte-dendritic cell progenitor (MDP) cells is decreased.

7. The method of any one of claims 1 to 6, wherein the at least one perturbagen selected from Table 3, or a variant thereof, comprises at least 2, at least 3, at least 4, at least 5, at least 6, at least 7, at least 8, at least 9, at least 10, at least 11, or 12 perturbagens selected from Table 3, or variants thereof, optionally wherein the at least one perturbagen promotes the monocyte lineage or the neutrophil lineage.

8. The method of any one of claims 1 to 7, wherein altering the gene signature comprises increased expression and/or increased activity in the progenitor cell of one or more genes selected from Table 1, optionally wherein the one or more genes selected from Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, or 49 genes selected from Table 1.

9. The method of claim 8, wherein the one or more genes selected from Table 1 comprises at least one of MYB, NCOA4, CEBPD, CEBPA, TLE4, DACH1, SMARCA2, GFI1, MAZ, TLE1, ELL2, and ARID1A, optionally at least one of MYB, NCOA4, CEBPD, CEBPA, and TLE4.

10. The method of any one of claims 1 to 9, wherein altering the gene signature comprises decreased expression and/or decreased activity in the progenitor cell of one or more genes selected from Table 2, optionally wherein the one or more genes selected from Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, or 72 genes selected from Table 2.

11. The method of claim 10, wherein the one or more genes selected from Table 2 comprises at least one of HMGA1, SSBP4, LSM4, CDK4, SMARCB1, AES, GTF3A, RAB8A, RNPS1, THRAP3, and SNRPB, optionally at least one of HMGA1, SSBP4, LSM4, CDK4, and SMARCB1.

12. A method for inhibiting a change in cell state of a progenitor cell comprising:
contacting a population of cells comprising a progenitor cell with at least one perturbagen selected from Table 6, or a variant thereof,
wherein the at least one perturbagen is capable of inhibiting a gene signature in the progenitor cell; and
wherein the progenitor cell is a non-lineage committed CD34+ cell.

13. A method for inhibiting a change in cell state of a progenitor cell, comprising:
contacting a population of cells comprising a progenitor cell with at least one perturbagen capable of altering a gene signature in the progenitor cell,
wherein inhibiting the gene signature comprises an increase in expression and/or activity in the progenitor cell of one or more genes selected from Table 4 and/or a decrease in expression and/or activity in the progenitor cell of one or more genes selected from Table 5 and wherein the progenitor cell is a non-lineage committed CD34+ cell.

14. A method for inhibiting a change in cell state of a progenitor cell, comprising:
contacting a population of cells comprising a progenitor cell with at least one perturbagen selected from Table 6, or a variant thereof, and capable of altering a gene signature in the progenitor cell,
wherein inhibiting the gene signature comprises an increase in expression and/or activity in the progenitor cell of one or more genes selected from Table 4 and/or a decrease in expression and/or activity in the progenitor cell of one or more genes selected from Table 5 and wherein the progenitor cell is a non-lineage committed CD34+ cell.

15. The method of any one of claims 12 to 14, wherein inhibiting the change in cell state provides one or more of the following:
a) a decrease in the number of neutrophils and/or the number of monocytes, optionally wherein the decrease in the number of neutrophils and/or the number of monocytes is relative to the number of neutrophils and/or the number of monocytes obtained from a population of progenitor cells that is not contacted with the at least one perturbagen or relative to the population of progenitor cells prior to contacting with the at least one perturbagen; and/or
b) the number of progenitor cells is increased, optionally wherein the increase in the number of progenitor cells is relative to the number of progenitor cells in a population of progenitor cells that is not contacted with the at least one perturbagen or relative to the number of progenitor cells in the population prior to contacting with the at least one perturbagen.

16. The method of any one of claims 12 to 15, wherein the number of CD66b+ neutrophil progenitors, CD64+monocytic progenitors, neutrophilic promyelocytes, neutrophilic myelocytes, neutrophilic metamyelocytes, and/or neutrophilic band cells is decreased and/or the number of CD34+CD38+/− cells is decreased about two days after contacting the population of cells comprising a CD34+ cell with the at least one perturbagen and/or the number of CD34-CD38+/− cells is decreased about five days after contacting the population of cells comprising a CD34+ cell with the at least one perturbagen.

17. The method of any one of claims 12 to 15, wherein the number of monoblasts and/or promonocytes is decreased and/or the number of granulocyte-monocyte progenitor (GMP) cells is decreased, optionally wherein the progenitor cell for the GMP cell is a common myeloid progenitor (CMP) and/or the number of monocyte-dendritic cell progenitor (MDP) cells is increased.

18. The method of any one of claims 12 to 17, wherein the at least one perturbagen selected from Table 6, or a variant thereof, comprises at least 2, at least 3, at least 4, at least 5, at least 6, at least 7, at least 8, at least 9, or 10 perturbagens selected from Table 6, or variants thereof.

19. The method of claim 18, wherein the at least one perturbagen prevents progression of a progenitor cell into and/or reduces the likelihood that a progenitor cell will progress into the monocyte lineage or the neutrophil lineage.

20. The method of any one of claims 12 to 19, wherein altering the gene signature comprises increased expression and/or increased activity in the progenitor cell of one or more genes selected from Table 4, optionally wherein the one or more genes selected from Table 4 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, or 72 genes selected from Table 4.

21. The method of claim 20, wherein the one or more genes selected from Table 4 comprises at least one of HMGA1, SSBP4, LSM4, CDK4, SMARCB1, AES, GTF3A, RAB8A, RNPS1, THRAP3, and SNRPB, optionally at least one of HMGA1, SSBP4, LSM4, CDK4, and SMARCB1.

22. The method of any one of claims 12 to 21, wherein altering the gene signature comprises decreased expression and/or decreased activity in the progenitor cell of one or more genes selected from Table 5, optionally wherein the one or more genes selected from Table 5 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, or 49 genes selected from Table 5.

23. The method of claim 22, wherein the one or more genes selected from Table 5 comprises at least one of MYB, NCOA4, CEBPD, CEBPA, TLE4, DACH1, SMARCA2, GFI1, MAZ, TLE1, ELL2, and ARID1A, optionally at least one of MYB, NCOA4, CEBPD, CEBPA, and TLE4.

24. The method of any one of claims 1 to 23, wherein contacting the population of progenitor cells occurs in vitro or ex vivo or in vivo in a subject.

25. A perturbagen for use in the method of any one of claims 1 to 24.

26. A pharmaceutical composition comprising the perturbagen of claim 25.

27. A method for promoting the formation of a neutrophil, a monocyte, or an immediate progenitor thereof, comprising:
(a) exposing a starting population of stem/progenitor cells comprising a non-lineage committed CD34+ cell to a perturbagen;
wherein the exposing promotes the transition of the starting population of stem/progenitor cells into a granulocyte monocyte progenitor, neutrophil progenitor, monocyte progenitor or other lineage associated progenitor thereof, or
(b) exposing a starting population of stem/progenitor cells comprising a non-lineage committed CD34+ cell to a perturbation having a perturbation signature that promotes the transition of the starting population of stem/progenitor cells into a granulocyte monocyte progenitor, neutrophil progenitor, monocyte progenitor or other lineage associated progenitor thereof,
wherein the perturbation signature comprises increased expression and/or activity of one or more of genes selected from Table 1 and/or a decreased expression and/or activity in the non-lineage committed CD34+ cell of one or more genes selected from Table 2.

28. A method for inhibiting the formation of a neutrophil, a monocyte, or an immediate progenitor thereof, comprising:
(a) exposing a starting population of stem/progenitor cells comprising a non-lineage committed CD34+ cell to a perturbation having a perturbation signature that prevents progression of a progenitor cell into and/or or reduces the likelihood that a progenitor cell will progress into a granulocyte monocyte progenitor, neutrophil progenitor, monocyte progenitor or other lineage associated progenitor thereof,
wherein the perturbation signature comprises increased expression and/or activity of one or more of genes selected from Table 4 and/or a decreased expression and/or activity in the non-lineage committed CD34+ cell of one or more genes selected from Table 5; or
(b) exposing a starting population of stem/progenitor cells comprising a non-lineage committed CD34+ cell to a perturbagen that prevents progression of a progenitor cell into and/or or reduces the likelihood that a progenitor cell will progress into a granulocyte monocyte progenitor, neutrophil progenitor, monocyte progenitor or other lineage associated progenitor thereof.

29. A method of increasing a quantity of neutrophils, monocytes, or immediate progenitors thereof, comprising:
exposing a starting population of stem/progenitor cells comprising a non-lineage committed CD34+ cell to a pharmaceutical composition that promotes the formation of lineage specific progenitor population selected from granulocyte monocyte progenitor, neutrophil progenitor, monocyte progenitor, or other lineage-associated progenitors, the pharmaceutical composition promoting the transition of a primitive stem/progenitor population into the lineage specific progenitor population that has the capacity to differentiate into neutrophils, monocytes or immediate progenitors thereof,
wherein the pharmaceutical composition comprises at least one perturbagen selected from Table 3, or a variant thereof.

30. A method of reducing the quantity of neutrophils, monocytes, or immediate progenitors thereof in a population of cells, comprising:
exposing a starting population of stem/progenitor cells comprising a non-lineage committed CD34+ cell to a pharmaceutical composition that prevents progression of a progenitor cell into and/or reduces the likelihood that a progenitor cell will progress into a lineage specific progenitor population selected from granulocyte monocyte progenitor, neutrophil progenitor, monocyte progenitor, or other lineage-associated progenitors, the pharmaceutical composition inhibiting the transition of a primitive stem/progenitor population into the lineage specific progenitor population that naturally would have the capacity to differentiate into neutrophils, monocytes or immediate progenitors thereof,
wherein the pharmaceutical composition comprises at least one perturbagen selected from Table 6, or a variant thereof.

31. A method for treating a disease or disorder characterized by an abnormal ratio of neutrophils to monocytes, comprising:
(a) administering to a patient in need thereof a therapeutically effective amount of at least one perturbagen selected from Table 3, or a variant thereof, wherein the at least one perturbagen is capable of changing a gene signature in a progenitor cell; or
(b) administering to a patient in need thereof a cell, the cell having been contacted with at least one perturbagen selected from Table 3, or a variant thereof, wherein the at least one perturbagen is capable of changing a gene signature in a progenitor cell;
(c) administering to a patient in need thereof a therapeutically effective amount of at least one perturbagen selected from Table 3, or a variant thereof, or
(d) administering to a patient in need thereof a cell, the cell having been contacted with at least one perturbagen selected from Table 3, or a variant thereof;
(e) administering to a patient in need thereof a therapeutically effective amount of at least one perturbagen selected from Table 6, or a variant thereof, wherein the at least one perturbagen is capable of changing a gene signature in a progenitor cell; or
(f) administering to a patient in need thereof a cell, the cell having been contacted with at least one perturbagen selected from Table 6, or a variant thereof, wherein the at least one perturbagen is capable of changing a gene signature in a progenitor cell;
(g) administering to a patient in need thereof a therapeutically effective amount of at least one perturbagen selected from Table 6, or a variant thereof, or
(h) administering to a patient in need thereof a cell, the cell having been contacted with at least one perturbagen selected from Table 6, or a variant thereof.

32. The method of claim 31, wherein the abnormal ratio comprises an increased number of neutrophils and/or a decreased number of monocytes, or a decreased number of neutrophils and/or an increased number of monocytes, or a decreased number of neutrophils and a decreased number of monocytes.

33. The method of claim 31, wherein the abnormal ratio comprises an increased number of neutrophils and/or an increased number of monocytes.

34. A method for treating a disease or disorder characterized by an abnormal ratio of neutrophils and/or monocytes to progenitor cells, comprising:
(a) administering to a patient in need thereof at least one perturbagen selected from Table 3, or a variant thereof, wherein the at least one perturbagen is capable of changing a gene signature in a progenitor cell or
(b) administering to a patient in need thereof a cell, the cell having been contacted with at least one perturbagen selected from Table 3, or a variant thereof, wherein the at least one perturbagen is capable of changing a gene signature in a progenitor cell.

35. The method of claim 34, wherein the abnormal ratio comprises a decreased number of neutrophils and/or a decreased number of monocytes and/or an increased number of progenitor cells; or a decreased number of neutrophils and an increased number of progenitor cells; or a decreased number of monocytes and an increased number of progenitor cells; or a decreased number of neutrophils and a decreased number of monocytes; or an increased number of progenitor cells.

36. The method of claim 34, wherein the abnormal ratio comprises an increased number of neutrophils and/or an increased number of monocytes and/or a decreased number of progenitor cells, optionally wherein the abnormal ratio comprises an increased number of neutrophils and an increased number of monocytes

37. The method of any one of claim 31, 32, 34, or 35, wherein the disease or disorder characterized by an abnormal ratio of neutrophils to monocytes and/or characterized by an abnormal ratio of neutrophils and/or monocytes to progenitor cells is selected from leukopenia, leukocytosis, agranulocytosis, granulocytosis, monocytopenia, monocytosis, neutropenia, and neutrophilia, leukemia (e.g., Chronic myeloid leukemia (CML), Chronic myelomonocytic leukemia (CMML), and Acute myeloblastic leukemia (AML), including AML-M5), cancer induced neutropenia, drug induced neutropenia, infection induce neutropenia, lupus (and other autoimmune disorders showing neutropenia), myelofibrosis, Chronic idiopathic neutropenia (CIN), and/or Autoimmune idiopathic neutropenia (AlN).

38. The method of any one of claim 31, 33, 34, or 36, wherein the disease or disorder characterized by an abnormal ratio of neutrophils to monocytes and/or characterized by an abnormal ratio of neutrophils and/or monocytes to progenitor cells is selected from leukocytosis, granulocytosis, monocytosis, and neutrophilia, leukemia (e.g., Chronic myeloid leukemia (CML), Chronic myelomonocytic leukemia (CMML), and Acute myeloblastic leukemia (AML), including AML-M5), cancer induced leukocytosis, drug induced leukocytosis, infection induced leukocytosis, lupus (and other autoimmune disorders showing leukocytosis), and myeloproliferative neoplasms (including polycythemia vera and myelofibrosis).

39. The method of any one of claims 31 to 38, wherein the disease or disorder characterized by an abnormal ratio of neutrophils to monocytes and/or characterized by an abnormal ratio of neutrophils and/or monocytes to progenitor cells is related to an autoimmune disease, a bone-marrow transplant, a cancer, a genetic disease, a bacterial infection, a viral infection, inflammatory bowel disease, tissue necrosis (e.g., myocardial infarction and burns), a previous or concurrent anti-cancer treatment, and/or, a previous or concurrent immune suppressive treatment.

40. A method for treating monocytopenia, comprising:
(a) administering to a patient in need thereof a therapeutically effective amount of at least one perturbagen selected from Table 3, or a variant thereof; or
(b) administering to a patient in need thereof a cell, the cell having been contacted with at least one perturbagen selected from Table 3, or a variant thereof.

41. A method for treating monocytosis, comprising:
(a) administering to a patient in need thereof a therapeutically effective amount of at least one perturbagen selected from Table 3, or a variant thereof; or
(b) administering to a patient in need thereof a cell, the cell having been contacted with at least one perturbagen selected from Table 3, or a variant thereof;
(c) administering to a patient in need thereof a therapeutically effective amount of at least one perturbagen selected from Table 6, or a variant thereof; or
(d) administering to a patient in need thereof a cell, the cell having been contacted with at least one perturbagen selected from Table 6, or a variant thereof.

42. A method for treating neutropenia, comprising:
(a) administering to a patient in need thereof a therapeutically effective amount of at least one perturbagen selected from Table 3, or a variant thereof; or
(b) administering to a patient in need thereof a cell, the cell having been contacted with at least one perturbagen selected from Table 3, or a variant thereof.

43. A method for treating neutrophilia, comprising:
(a) administering to a patient in need thereof a therapeutically effective amount of at least one perturbagen selected from Table 3, or a variant thereof; or
(b) administering to a patient in need thereof a cell, the cell having been contacted with at least one perturbagen selected from Table 3, or a variant thereof; or
(c) administering to a patient in need thereof a therapeutically effective amount of at least one perturbagen selected from Table 6, or a variant thereof; or
(d) administering to a patient in need thereof a cell, the cell having been contacted with at least one perturbagen selected from Table 6, or a variant thereof.

44. A method for treating agranulocytosis, comprising:
(a) administering to a patient in need thereof a therapeutically effective amount of at least one perturbagen selected from Table 3, or a variant thereof; or
(b) administering to a patient in need thereof a cell, the cell having been contacted with at least one perturbagen selected from Table 3, or a variant thereof, optionally wherein the agranulocytosis is monocytopenia or neutropenia.

45. A method for treating granulocytosis, comprising:
(a) administering to a patient in need thereof a therapeutically effective amount of at least one perturbagen selected from Table 3, or a variant thereof; or
(b) administering to a patient in need thereof a cell, the cell having been contacted with at least one perturbagen selected from Table 3, or a variant thereof, optionally wherein the agranulocytosis is monocytopenia or neutropenia; or
(c) administering to a patient in need thereof a therapeutically effective amount of at least one perturbagen selected from Table 6, or a variant thereof; or
(d) administering to a patient in need thereof a cell, the cell having been contacted with at least one perturbagen selected from Table 6, or a variant thereof.

46. A method for treating a myeloproliferative neoplasm, comprising:
(a) administering to a patient in need thereof a therapeutically effective amount of at least one perturbagen selected from Table 6, or a variant thereof; or
(b) administering to a patient in need thereof a cell, the cell having been contacted with at least one perturbagen selected from Table 6, or a variant thereof

47. The method of any one of claims 40 to 46, wherein the at least one perturbagen is capable of changing a gene signature in a progenitor cell.

48. A method of treating a disorder selected from drug-induced neutropenia, a side-effect of bone marrow transplantation, or MonoMAC syndrome, comprising:
(a) administering to a patient in need thereof a therapeutically effective amount of at least one perturbagen selected from Table 3, or a variant thereof; or
(b) administering to a patient in need thereof a cell, the cell having been contacted with at least one perturbagen selected from Table 3, or a variant thereof.

49. The method of any one of claims 40 to 48, wherein the therapeutically effective amount of the at least one perturbagen provides a change in a gene signature in a cell,
wherein the change in the gene signature comprises an increase in expression and/or activity in the cell of one or more genes selected from Table 1 and/or a decrease in expression and/or activity in the cell of one or more genes selected from Table 2 and/or an increase in expression and/or activity in the cell of one or more genes selected from Table 4 and/or a decrease in expression and/or activity in the cell of one or more genes selected from Table 5.

50. The method of any one of claims 40 to 49, wherein the therapeutically effective amount of the at least one perturbagen provides an increase in the number of neutrophils and/or the number of monocytes in a sample from the human subject relative to the number of neutrophils and/or the number of monocytes in a sample from the human subject prior to administering the at least one perturbagen.

51. The method of any one of claims 40 to 49, wherein the therapeutically effective amount of the at least one perturbagen provides a decrease in the number of neutrophils and/or the number of monocytes in a sample from the human subject relative to the number of neutrophils and/or the number of monocytes in a sample from the human subject prior to administering the at least one perturbagen.

52. The method of any one of claims 31 to 51, wherein the patient was selected by steps comprising:
(a) obtaining from the patient having the disease or disorder a sample of cells comprising a non-lineage committed CD34+ cell; and
contacting the sample of cells with least one perturbagen selected from Table 3 or Table 6, or a variant thereof wherein the at least one perturbagen alters a gene signature in the sample of cells; or
(b) obtaining from a subject having the disease or disorder a sample of cells comprising a non-lineage committed CD34+ cell; and
contacting the sample of cells with at least one perturbagen capable of altering a gene signature in a non-lineage committed CD34+ cell,
wherein the at least one perturbagen increases in the sample of cells the expression and/or activity of one or more genes selected from Table 1 and/or decreases in the sample of cells the expression and/or activity of one or more genes selected from Table 2 and/or increases in the sample of cells the expression and/or activity of one or more genes selected from Table 4 and/or decreases in the sample of cells the expression and/or activity of one or more genes selected from Table 5; or
(c) obtaining from a subject having the disease or disorder a sample of cells comprising a non-lineage committed CD34+ cell; and
contacting the sample of cells with at least one perturbagen selected from Table 3 or Table 6, or a variant thereof;
wherein the at least one perturbagen increases in the sample of cells the expression and/or activity of one or more genes selected from Table 1 and/or decreases in the sample of cells the expression and/or activity of one or more genes selected from Table 2; or
increases in the sample of cells the expression and/or activity of one or more genes selected from Table 4 and/or decreases in the sample of cells the expression and/or activity of one or more genes selected from Table 5.

53. A method for selecting the patient of any one of claims 31 to 51 comprising:
obtaining from a subject having the disease or disorder a sample of cells comprising a non-lineage committed CD34+ cell; and
contacting the sample of cells with least one perturbagen selected from Table 3 or Table 6, or a variant thereof, wherein when the at least one perturbagen alters a gene signature in the sample of cells, the subject is selected as a patient.

54. A method for selecting the patient of any one of claims 31 to 51 comprising:
obtaining from a subject having the disease or disorder a sample of cells comprising a non-lineage committed CD34+ cell; and
contacting the sample of cells with at least one perturbagen capable of altering a gene signature in a non-lineage committed CD34+ cell,
wherein when the at least one perturbagen increases in the sample of cells the expression and/or activity of one or more genes selected from Table 1 and/or decreases in the sample of cells the expression and/or activity of one or more genes selected from Table 2 and/or increases in the sample of cells the expression and/or activity of one or more genes selected from Table 4 and/or decreases in the sample of cells the expression and/or activity of one or more genes selected from Table 5, the subject is selected as a patient.

55. A method for selecting the patient of any one of claims 31 to 51 comprising:
obtaining from a subject having the disease or disorder a sample of cells comprising a non-lineage committed CD34+ cell; and
contacting the sample of cells with at least one perturbagen selected from Table 3 or Table 6, or a variant thereof;
wherein when the at least one perturbagen increases in the sample of cells the expression and/or activity of one or more genes selected from Table 1 and/or decreases in the sample of cells the expression and/or activity of one or more genes selected from Table 2; or
increases in the sample of cells the expression and/or activity of one or more genes selected from Table 4 and/or decreases in the sample of cells the expression and/or activity of one or more genes selected from Table 5, the subject is selected as a patient.

56. The method of any one of claims 31 to 51, wherein the patient was selected by steps comprising:
(a) obtaining from the patient having the disease or disorder a sample of cells comprising a non-lineage committed CD34+ cell; and
contacting the sample of cells with least one perturbagen selected from Table 3, or a variant thereof, wherein the at least one perturbagen increases the number of monocytes and/or neutrophils or a progenitor thereof in a culture from the sample of cells; or
(b) obtaining from a subject having the disease or disorder a sample of cells comprising a non-lineage committed CD34+ cell; and
contacting the sample of cells with least one perturbagen selected from Table 3, or a variant thereof, wherein when the at least one perturbagen increases the number of monocytes and/or neutrophils or a progenitor thereof in a culture from the sample of cells, the subject is selected as a patient.

57. The method of any one of claims 31 to 51, wherein the patient was selected by steps comprising:
(a) obtaining from the patient having the disease or disorder a sample of cells comprising a non-lineage committed CD34+ cell; and
contacting the sample of cells with least one perturbagen selected from Table 6, or a variant thereof, wherein the at least one perturbagen increases the number of monocytes and/or neutrophils or a progenitor thereof in a culture from the sample of cells; or
(b) obtaining from a subject having the disease or disorder a sample of cells comprising a non-lineage committed CD34+ cell; and
contacting the sample of cells with least one perturbagen selected from Table 6, or a variant thereof, wherein when the at least one perturbagen increases the number of monocytes and/or neutrophils or a progenitor thereof in a culture from the sample of cells, the subject is selected as a patient.

58. Use of the perturbagen of Table 3 or Table 6, or a variant thereof in the manufacture of a medicament for treating a disease or disorder characterized by an abnormal ratio of neutrophils to monocytes.

59. Use of the perturbagen of Table 3 or Table 6, or a variant thereof in the manufacture of a medicament for treating a disease or disorder characterized by an abnormal ratio of neutrophils and/or monocytes to progenitor cells.

60. A method of identifying a candidate perturbation for promoting the transition of a starting population of progenitor cells into neutrophils, monocytes or immediate progenitors thereof, the method comprising:
(a) exposing the starting population of progenitor cells to a perturbation;
identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into neutrophils, monocytes or immediate progenitors thereof following exposure of the population of cells to the perturbation; and
identifying the perturbation as a candidate perturbation for promoting the transition of a population of progenitor cells into neutrophils, monocytes or immediate progenitors thereof based on the perturbation signature, wherein the perturbation signature is an increase in expression and/or activity in the progenitor cell of one or more genes selected from Table 1, and/or a decrease in expression and/or activity in the progenitor cell of one or more genes selected from Table 2; or
(b) exposing the starting population of progenitor cells to a candidate perturbagen;
determining a change in cell state of the cells in the population of progenitor cells into neutrophils, monocytes or immediate progenitors thereof following exposure of the population of cells to the perturbagen wherein the candidate perturbagen capable changing the cell state of the cells in the population progenitor cells is identified as a perturbagen for promoting the transition of a starting population of progenitor cells into neutrophils, monocytes or immediate progenitors thereof.

61. A method of identifying a candidate perturbation for inhibiting the transition of a progenitor cell into a neutrophil, monocyte or immediate progenitor thereof, the method comprising:
exposing a starting population of progenitor cells to a perturbation;
identifying a perturbation signature for the perturbation, the perturbation signature comprising one or more cellular-components and a significance score associated with each cellular-component, the significance score of each cellular-component quantifying an association between a change in expression of the cellular-component and preventing progression of a progenitor cell into and/or reducing the likelihood that a progenitor cell will progress into a neutrophil, monocyte or immediate progenitor thereof following exposure of the population of progenitor cells to the perturbation; and
identifying the perturbation as a candidate perturbation for inhibiting the transition of a progenitor cell into a neutrophil, monocyte or immediate progenitor thereof based on the perturbation signature,
wherein the perturbation signature is an increase in expression and/or activity in the progenitor cell of one or more genes selected from Table 4, and/or a decrease in expression and/or activity in the progenitor cell of one or more genes selected from Table 5.

62. A method of identifying a perturbagen, the method comprising:
exposing a starting population of progenitor cells, which naturally would have the capacity to undergo a change in cell state comprising differentiating into neutrophils, monocytes and/or immediate progenitors thereof, to a candidate perturbagen; and
determining an inhibition in the natural capacity to differentiate into neutrophils, monocytes and/or immediate progenitors thereof following exposure of the population of cells to the perturbagen;
wherein the candidate perturbagen capable of inhibiting the natural capacity of a progenitor cell to differentiate into a neutrophil, monocyte and/or immediate progenitor thereof is identified as a perturbagen.

63. A method for making a therapeutic agent for a disease or disorder selected from leukocytosis, granulocytosis, monocytosis, and neutrophilia, leukemia, cancer induced leukocytosis, drug induced leukocytosis, infection induce leukocytosis, lupus (and other autoimmune disorders showing leukocytosis), and myeloproliferative neoplasms (including polycythemia vera and myelofibrosis), comprising:
(a) identifying a perturbagen capable of acting as a therapeutic agent for therapy according to the method of claim 62 and
(b) formulating the therapeutic agent for the treatment of the disease or disorder.

64. A method for making a therapeutic agent for a disease or disorder selected from leukopenia, leukocytosis, agranulocytosis, granulocytosis, monocytopenia, monocytosis, neutropenia, and neutrophilia, leukemia, cancer induced neutropenia, drug induced neutropenia, infection induce neutropenia, lupus (and other autoimmune disorders showing neutropenia), myelofibrosis, Chronic idiopathic neutropenia (CIN), and Autoimmune idiopathic neutropenia (AlN), comprising:
(a) identifying a therapeutic agent for therapy according to the method of claim 60 and
(b) formulating the therapeutic agent for the treatment of the disease or disorder.

65. A method for making a therapeutic agent for a disease or disorder selected from leukocytosis, granulocytosis, monocytosis, and neutrophilia, leukemia, cancer induced leukocytosis, drug induced leukocytosis, infection induce leukocytosis, lupus (and other autoimmune disorders showing leukocytosis), and myeloproliferative neoplasms (including polycythemia vera and myelofibrosis), comprising:
(a) identifying a therapeutic agent for therapy according to the method of claim 61 and
(b) formulating the therapeutic agent for the treatment of the disease or disorder.

66. A method for directing a change in cell state of a progenitor cell, comprising:
contacting a population of cells comprising a progenitor cell with at least one perturbagen selected from Table 3, or a variant thereof, and capable of altering a gene signature in the progenitor cell,
wherein altering the gene signature comprises an increase in expression and/or activity in the progenitor cell of one or more genes selected from Table 1 and/or a decrease in expression and/or activity in the progenitor cell of one or more genes selected from Table 2 and wherein the progenitor cell is a non-lineage committed CD34+ cell; and
the change in cell state provides an increase in the number of CD66b+ neutrophil progenitors and an increase in the number of CD64+monocytic progenitors.

67. The method of claim 66, wherein the increase in the number of CD66b+ neutrophil progenitors and the increase in the number of CD64+monocytic progenitors is relative to the population of progenitor cells not contacted with the at least one perturbagen.

68. The method of claim 66, wherein the one or more genes selected from Table 1 comprises at least one of MYB, NCOA4, CEBPD, CEBPA, and TLE4.

69. The method of claim 66, wherein the one or more genes selected from Table 2 comprises at least one of HMGA1, SSBP4, LSM4, CDK4, and SMARCB1.

70. A method for inhibiting a change in cell state of a progenitor cell, comprising:
contacting a population of cells comprising a progenitor cell with at least one perturbagen selected from Table 6, or a variant thereof, and capable of altering a gene signature in the progenitor cell,
wherein inhibiting the gene signature comprises an increase in expression and/or activity in the progenitor cell of one or more genes selected from Table 4 and/or a decrease in expression and/or activity in the progenitor cell of one or more genes selected from Table 5 and
wherein the progenitor cell is a non-lineage committed CD34+ cell, and
the change in cell state provides a decrease in the number of CD66b+ neutrophil progenitors and CD64+monocytic progenitors.

71. The method of claim 70, wherein the decrease in the number of CD66b+ neutrophil progenitors and the decrease in the number of CD64+monocytic progenitors is relative to the number of progenitor cells in a population of progenitor cells that is not contacted with the at least one perturbagen.

72. The method of claim 70, wherein the one or more genes selected from Table 4 comprises at least one of HMGA1, SSBP4, LSM4, CDK4, and SMARCB1.

73. The method of claim 70, wherein the one or more genes selected from Table 5 comprises at least one of MYB, NCOA4, CEBPD, CEBPA, and TLE4.
